SEQUENCE LISTING (65 sequences)

SEQ ID NO: 1 - protein, 606 aa, Drosophila sechellia
RPEPPVNSYLPPSDSYGAPGQSGAGGRPSDTYGAPGGGNGGRPSDSYGAPGQGQGQGQGQGGYGGKPSDSYGAPGGGNGNGGRPSSSYGAPGGGNGGRPSDTYGAPGGGNGGRPSDTYGAPGGGGNGNGGRPSSSYGAPGQGQGNGNGGRPSSSYGAPGGGNGGRPSDTYGAPGGGNGGRPSDTYGAPGGGNNGGRPSSSYGAPGGGNGGRPSDTYGAPGGGNGNGSGGRPSSSYGAPAQGQGGFGGRPSDSYGAPGQNQKPSDSYGAPGSGNGSAGRPSSSYGAPGSGPGGRPSDSYGPPASGSGAGGAGGSGPGGADYDNDEPAKYEFNYQVEDAPSGLSFGHSEMRDGDFTTGQYNVLLPDGRKQIVEYEADQQGYRPQIRYEGDANDGSGPSGPSGPGGPGGQNLGADGYSSGRPGNGNGNGNGGYSSGRPGGQDLGPSGYSGGRPGGQDLGAGGYSNVKPGGQDLGPGGYSGGRPGGQDLGRDGYSGGRPGGQDLGAGAYSNGRPGGNGNGGSDGGRVIIGGRVIGGQDGGDQGYSGGRPGGQDLGRDGYSSGRPGGRPGGNGQDSQDGQGYSSGRPGQGGRNGFGPGGQNGDNDGSGYRY

SEQ ID NO: 2 - protein, 304 aa, Drosophila sechellia
RPEPPVNSYLPPSDSYGAPGQSGAGGRPSDTYGAPGGGNGGRPSDSYGAPGQGQGQGQGQGGYGGKPSDSYGAPGGGNGNGGRPSSSYGAPGGGNGGRPSDTYGAPGGGNGGRPSDTYGAPGGGGNGNGGRPSSSYGAPGQGQGNGNGGRPSSSYGAPGGGNGGRPSDTYGAPGGGNGGRPSDTYGAPGGGNNGGRPSSSYGAPGGGNGGRPSDTYGAPGGGNGNGSGGRPSSSYGAPAQGQGGFGGRPSDSYGAPGQNQKPSDSYGAPGSGNGSAGRPSSSYGAPGSGPGGRPSDSYGPPASG

SEQ ID NO: 3 - protein, 211 aa, Drosophila sechellia
GKPSDSYGAPGGGNGNGGRPSSSYGAPGGGNGGRPSDTYGAPGGGNGGRPSDTYGAPGGGGNGNGGRPSSSYGAPGQGQGNGNGGRPSSSYGAPGGGNGGRPSDTYGAPGGGNGGRPSDTYGAPGGGNNGGRPSSSYGAPGGGNGGRPSDTYGAPGGGNGNGSGGRPSSSYGAPAQGQGGFGGRPSDSYGAPGQNQKPSDSYGAPGSGNGS

SEQ ID NO: 4 - protein, 175 aa, Acromyrmex echinatior
FGENRGNGGKPSTSYGVPDSNGNNRGGFGNGGSEGRPSTSYGLPDASRNNGNGFGNVGNEDKPSTNYGIPANGNKVSGFGNVGSEGRPSTSYGVPGANGNQGFGSGGIGGRPSTSYGVPGVNGNNGGGFENVGRPSTSYGTPDARGNNGGSFRNGDIGGRPSTNYGIPGANGNHG

SEQ ID NO: 5 - protein, 158 aa, Aeshna sp.
APSRGGGHGGGSISSSYGAPSKGSGGFGGGSISSSYGAPSKGSVGGGVSSSYGAPAIGGGSFGGGSFGGGSFGGGSFGGGAPSSSYGAPSSSYSAPSSSYGAPSKGSGGFGSSGGFSSFSSAPSSSYGAPSASYSTPSSSYGAPSSGGFGAGGGFSSG

SEQ ID NO: 6 - protein, 457 aa, Aeshna sp.
EPPVGGSQSYLPPSSSYGAPSAGTGFGHGGGSPSQSYGAPSFGGGSVGGGSHFGGGSHSGGGGGGYPSQSYGAPSRPSGSSFQAFGGAPSSSYGAPSSQYGAPSGGGGSYAIQGGSFSSGGSRAPSQAYGAPSNNAGLSHQSQSFGGGLSSSYGAPSAGFGGQSHGGGYSQGGNGGGHGGSSGGGYSYQSFGGGNGGGHGGSRPSSSYGAPSSSYGAPSGGKGVSGGFVSQPSGSYGAPSQSYGAPSRGGGHGGGSISSSYGAPSKGSGGFGGGSISSSYGAPSKGSVGGGVSSSYGAPAIGGGSFGGGSFGGGSFGGGSFGGGAPSSSYGAPSSSYSAPSSSYGAPSKGSGGFGSSGGFSSFSSAPSSSYGAPSASYSTPSSSYGAPSSGGFGAGGGFSSGGYSGGGGGYSSGGSGGFGGHGGSGGAGGYSGGGGYSGGGSGGGQKYDSNGGYVYS

SEQ ID NO: 7 - protein, 162 aa, Haematobia irritans
AGGGNGGGGTGGTPSSSYGAPSNGGGSNGNGFGSPSSSYGAPGSGGSNGNGGGRPSLSYGAPGSGGSNGNGGGRPSSSYGAPGAGGSNGNGGGRPSSSYGAPGAGGSNGNGGGRPSSSYGAPGAGGSNGNGGGRPSSSYGAPGAGGSNGNGGSRPSSTYGAP

SEQ ID NO: 8 - protein, 580 aa, Haematobia irritans
RPEPPVNSYLPPPLNNYGAPGAGGGSSDGSPLAPSDAYGAPDLGGGSGGSGQGPSSSYGAPGLGGGNGGAPSSSYGAPGLGGGNGGSRRPSSSYGAPGAGGGNGGGGTGGTPSSSYGAPSNGGGSNGNGFGSPSSSYGAPGSGGSNGNGGGRPSLSYGAPGSGGSNGNGGGRPSSSYGAPGAGGSNGNGGGRPSSSYGAPGAGGSNGNGGGRPSSSYGAPGAGGSNGNGGGRPSSSYGAPGAGGSNGNGGSRPSSTYGAPGAGGSNGNGCGNKPSSSYGAPSAGSNGNGGSEQGSSGSPSDSYGPPASGTGRGRNGGGGGAGGGRRGQPNQEYLPPNQGDNGNNGGSGGDDGYDYSQSGDGGGQGGSGGSGNGGDDGSNIVEYEAGQEGYRPQIRYEGEANEGGQGSGGAGGSDGTDGYEYEQNGGDGGAGGSGGPGTGQDLGENGYSSGRPGGDNGGGGGYSNGNGQGDGGQDLGSNGYSSGAPNGQNGGRRNGGGQNNNGQGYSSGRPNGNGSGGRNGNGGRGNGGGYRNGNGNGGGNGNGSGSGSGNNGYNYDQQGSNGFGAGGQNGENDGSGYRYS

SEQ ID NO: 9 - protein, 256 aa, Ctenocephalides felis
ANGNGFEGASNGLSATYGAPNGGGFGGNGNGGAPSSSYGAPGAGNGGNGGGRPSSSYGAPGAGGSGNGFGGRPSSSYGAPGNGNGANGGRGGRPSSRYGAPGNGNGNGNGNGGRPSSSYGAPGSNGNGGRPSSSYGAPGSGNGFGGNGGRPSSSYGAPGANGNGNGGAIGQPSSSYGAPGQNGNGGGLSSTYGAPGAGNGGFGGNGGGLSSTYGAPGSGNGGFGGNGLSSTYGAPGSGNGGFGGNGGGLSSTYGAP

SEQ ID NO: 10 - protein, 146 aa, Ctenocephalides felis
PGGAGGAGGYPGGAGGAGGAGGYPGGSAGGAGGYPGGSGSGVGGYPGGSNGGAGGYPGGSNGGAGGYPGGSNGGAGGYPGGSNGGAGGYPGGSNGNGGYSNGGSNGGGAGGYPGGSNGNGGYPGSGSNGGAGGYPGGSNGNGGYPG

SEQ ID NO: 11 - protein, 201 aa, Bombus terrestris
FDGQNGIGGGDSGRNGLSNSYGVPGSNGGRNGNGRGNGFGGGQPSSSYGAPSNGLGGNGGSGAGRPSSSYGAPGGGNGFGGGQPSSSYGAPSNGLGGNGAGRPSSSYGAPGGGNGFGGGSNGAGKNGFGGAPSNSYGPPENGNGFGGGNGGGSPSGLYGPPGRNGGNGGNGGNGGNGGRPSSSYGTPERNGGRPSGLYGPP

SEQ ID NO: 12 - protein, 183 aa, Tribolium castaneum
NGFGGGQNGGRLSSTYGPPGQGGNGFGGGQNGGRPSSTYGPPGQGGNGFGGGQNGGRPSSTYGPPGQGGNGFGGGQNGGRPSSTYGPPGQGGNGFGGGQNGGRPSSTYGPPGQGGNGFGGGQNGGRPSSTYGPPGQGGNGFGGGQNGGKPSSTYGPPGQGGNGFGGGQNGGRPSSTYGPPGQG

SEQ ID NO: 13 - protein, 551 aa, Tribolium castaneum
RAEPPVNSYLPPSQNGGPSSTYGPPGFQPGTPLGGGGNGGHPPSQGGNGGFGGRHPDSDQRPGTSYLPPGQNGGAGRPGVTYGPPGQGGGQNGGGPSSTYGPPGQGGNGFGGGQNGGRLSSTYGPPGQGGNGFGGGQNGGRPSSTYGPPGQGGNGFGGGQNGGRPSSTYGPPGQGGNGFGGGQNGGRPSSTYGPPGQGGNGFGGGQNGGRPSSTYGPPGQGGNGFGGGQNGGRPSSTYGPPGQGGNGFGGGQNGGKPSSTYGPPGQGGNGFGGGQNGGRPSSTYGPPGQGGNGNGGGHNGQRPGGSYLPPSQGGNGGYPSGGPGGYPSGGPGGNGGYGGEEESTEPAKYEFEYQVDDDEHNTHFGHQESRDGDKATGEYNVLLPDGRKQVVQYEADSEGYKPKISYEGGNGNGGYPSGGPGGAGNGGYPSGGPQGGNGGYPSGGPQGGNGGYPSGGPQGGNGGYPSGGPQGGNGGYPSGGPQGGNGGYPSGGPQGGNGGYPSGGPQGGNGGYPSGGPQGGNGGYTSGGPQGGNGGYPSGGPQGGNGGSGPY

SEQ ID NO: 14 - protein, 444 aa, Tribolium castaneum
QLTKRDAPLSGGYPSGGPANSYLPPGGASQPSGNYGAPSGGFGGKSGGFGGSGGFGGAPSQSYGAPSGGFGGSSSFGKSGGFGGAPSQSYGAPSGGFGGSSSFGKSSGGFGGAPSQSYGAPSGGFGGSSSFGKSGGFGGAPSQSYGAPSGGFGGSSSFGKSGGFGGAPSQSYGAPSGGFGGKSSSFSSAPSQSYGAPSGGFGGKSGGFGGAPSQSYGAPSGGFGGKSGGFGGAPSQSYGAPSGGFGGSSSFGKSGGFGGAPSQSYGAPSGGFGGSSSFGKSSGFGHGSGAPSQSYGAPSRSQPQSNYLPPSTSYGTPVSSAKSSGSFGGAPSQSYGAPSQSHAPSQSYGAPSRSFSQAPSQSYGAPSQGHAPAPQQSYSAPSQSYGAPSGGFGGGHGGFGGQGQGFGGGRSQPSQSYGAPAPSQSYGAPSAGGQQYASNGGYSY

SEQ ID NO: 15 - protein, 426 aa, Apis mellifera
RSEPPVNSYLPPSGNGNGGGGGGSSNVYGPPGFDGQNGIGEGDNGRNGISNSYGVPTGGNGYNGDSSGNGRPGTNGGRNGNGNGRGNGYGGGQPSNSYGPPSNGHGGNGAGRPSSSYGAPGGGNGFAGGSNGKNGFGGGPSSSYGPPENGNGFNGGNGGPSGLYGPPGRNGGNGGNGGNGGRPSGSYGTPERNGGRLGGLYGAPGRNGNNGGNGYPSGGLNGGNGGYPSGGPGNGGANGGYPSGGSNGDNGGYPSGGPNGNGNGNGGYGQDENNEPAKYEFSYEVKDEQSGADYGHTESRDGDRAQGEFNVLLPDGRKQIVEYEADQDGFKPQIRYEGEANSQGYGSGGPGGNGGDNGYPSGGPGGNGYSSGRPNGGSDFSDGGYPSTRPGGENGGYRNGNNGGNGNGGYPSGNGGDAAANGGYQY

SEQ ID NO: 16 - protein, 318 aa, Apis mellifera
DAPISGSYLPPSTSYGTPNLGGGGPSSTYGAPSGGGGGRPSSSYGAPSSTYGAPSSTYGAPSNGGGRPSSTYGAPSNGGGRPSSSYGAPSSSYGAPSSTYGAPSNGGGRPSSSYGAPSFGGGGGFGGGNGLSTSYGAPSRGGGGGGGSISSSYGAPTGGGGGGPSTTYGAPNGGGNGYSRPSSTYGTPSTGGGSFGGSGGYSGGGGGYSGGGNGYSGGGGGGYSGGNGGGYSGGGNGGGYSGGNGGGYSGGGGGGYSGGGGGGYSGGGNGYSGGGGGGYSGGNGGYSGGNGGYSGGGGGYSGGGGGGQSYASNGGYQY

SEQ ID NO: 17 - protein, 747 aa, Nasonia vitripennis
RPEPPVNSYLPPGQGGGFGGGRPSGASPSDQYGPPDFQGAGGRGGQAAGGNFGGGGNGFGGAPSSSYGPPGFGSNEPNKFSGAGGGGAGRPQDSYGPPAGGNGFAGSAGAGNSGRPGGAAAGGRPSDSYGPPQGGGSGFGGGNAGRPSDSYGPPSAGGGGFGGGSPGGGFGGGSPGGGFGGGNQGAPQSSYGPPASGFGGQGGAGQGRPSDSYGPPGGGSGGRPSQGGNGFGGGNAGRPSDSYGPPAAGGGGFGGNAGGNGGGNGFGGGRPSGSPGGFGGQGGGGRPSDSYLPPSGGSGFGGGNGRQPGGFGQQGGNGAGQQNGGGGAGRPSSSYGPPSNGNGGGFSGQNGGRGSPSSGGGFGGAGGSPSSSYGPPAGGSGFGNNGGAGGRPSSSYGPPSSGGNGFGSGGQGGQGGQGGQGGRPSSSYGPPSNGNGGFGGGNGGRPSSNGYPQGQGNGNGGFGGQGGNGGRPSSSYGPPGGDSGYPSGGPSGNFGGSNAGGGGGGFGGQVQDSYGPPPSGAVNGNGNGYSSGGPGGNGLDEGNDEPAKYEFSYEVKDDQSDGRKQIVEYEADQDGFKPQIRYEGEANTGAGGAGGYPSGGGGDSGYPSGPSGAGGNAGYPSGGGGGAGGFGGNGGGSNGYPSGGPSGGQGQFGGQQGGNGGYPSGPQGGSGFGGGSQGSGSGGYPSGGPGGNGGNNNFGGGNAGYPSGGPSGGNGFNQGGQNQGGSGGGYPSGSGGDAAANGGYQYS

SEQ ID NO: 18 - protein, 419 aa, Nasonia vitripennis
RAEAPISGNYLPPSTSYGTPNLGGGGGGGGGFGGGAPSSSYGAPSSGGGFGGSFGGGAPSSSYGAPSTGGSFGGGAPSSSYGAPSSGGSFGGSFGGGAPSSSYGAPSFGGNAPSSSYGAPSAGGSFGGGAPSNSYGPPSSSYGAPSAGGSFGGSSGGSFGGSFGGGAPSSSYGAPAPSRPSSNYGAPSRPSSNYGAPSSGGSGFGGGSGFGGGRPSSSYGAPSSGSFGGGFGGGAPSSSYGAPAPSRPSSNYGAPAPSRPSSNYGAPAPSRPSSSYGAPSRPSSNYGAPSRPSSNYGAPSSGGSGFGGGSGFGGGRPSSSYGAPSSGSFGGGFGGGAPSSSYGAPAPSRPSSNYGPPSSSYGAPSSGGSGGFGGGAPSSSYGAPSFGGSSNAVSRPSSSYGAPSSGGGQSYASNGGYQY

SEQ ID NO: 19 - protein, 396 aa, Pediculus humanus
EPPVKTSYLPPSASRSLNSQYGAPAFTDSNELVAPSPNSNFHDSYNQQQQSFDLSNGLSVPSAAGRLSNTYGVPSAQGANVPSFDSSDSIAVDAAGRSGNSFSSHVPSSTYGAPGNGFGGGSRSSQSGAPSSVYGPPQARNNNFGNGAAPSSVYGPPQARNNNFGNGGAPSQVYGPPKARNNNFGNGAAPSSVYGPPQARNNNFGNGAAPSSVYGPPQARNNNFANSAAPSQVYGPPQARNNNFGNGAAPSSVYGPPQSSSFSSPSGRSGQLPSATYGAPFERNGFGSQGSSGFQGYEPSKRSQTTEDPFAEPAKYEYDYKVQASDETGTEFGHKESRENESARGAYHVLLPDGRMQIVQYEADETGYRPQIRYEDTGYPSAASSRSNNGFNGYQY

SEQ ID NO: 20 - protein, 573 aa, Anopheles gambiae
KREAPLPPSGSYLPPSGGAGGYPAAQTPSSSYGAPTGGAGSWGGNGGNGGRGHSNGGGSSFGGSAPSAPSQSYGAPSFGGQSSGGFGGHSSGGFGGHSSGGHGGNGNGNGNGYSSGRPSSQYGPPQQQQQQQSFRPPSTSYGVPAAPSQSYGAPAQQHSNGGNGGYSSGRPSTQYGAPAQSNGNGFGNGRPSSSYGAPARPSTQYGAPSAGNGNGYAGNGNGRSYSNGNGNGHGNGHSNGNGNNGYSRGPARQPSQQYGPPAQAPSSQYGAPAQTPSSQYGAPAQTPSSQYGAPAQTPSSQYGAPAQTPSSQYGAPAPSRPSQQYGAPAPSRPSQQYGAPAQTPSSQYGAPAQTPSSQYGAPAQTPSSQYGAPAQTPSSQYGAPAQQPSSQYGAPAPSRPSQQYGAPAQQPSAQYGAPAQTPSSQYGAPAPSRPSQQYGAPAQAPSSQYGAPAPSSQYGAPAQQPSSQYGAPAQTPSSQYGAPSFGPTGGASFSSGNGNVGGSYQVSSTGNGFSQASFSASSFSPNGRTSLSAGGFSSGAPSAQSAGGYSSGGPSQVPATLPQSYSSNGGYNY

SEQ ID NO: 21 - protein, 513 aa, Glossina morsitans
RPEPPVNTYLPPSAGGGSGGGSPLAPSDTYGAPGVNGGGGGGGGPSSTYGAPGSGGGNGNGGGGFGKPSSTYGAPGLGGGGNGGGRPSETYGAPSGGGGNGFGKPSSTYGAPNGGGGNGGPGRPSSTYGAPGSGGGNGGSGRPSSTYGAPGLGGGNGGSGRPSSMYGAPGLGGGNGGSGRPSSTYGAPGSGGGNGGSGRPSSTYGAPGSGGGNGGSGRPSSTYGAPGNGNGGNGFGRPSSTYGAPGSGGSNGNGKPSSTYGAPGSGGGGGRPSDSYGPPASGNGGRNGNGNGQSQEYLPPGQSGSGGGGGYGGGSGSGGSGGGGGGGYGGDQDNNVVEYEADQEGYRPQIRYEGDGSQGGFGGDGDGYSYEQNGVGGDGGGAGGAGGYSNGQNLGANGYSSGRPNGGNGGGRRGGGGGGGGSGGGQNLGSNGYSSGAPNGFGGGNGQGYSGGRSNGNGGGGGGRNGGRYRNGGGGGGGRNGGGSNGYNYDQPGSNGFGRGGGNGENDGSGYHY

SEQ ID NO: 22 - protein, 506 aa, Atta cephalotes
RSEPPVNSYLHPGSDTSGTNGGRTDLSTQYGAPDFNNRGNGNSGATSFGGSGAGNGPSKLYDVPIRGNTGGNGLGQFRGNGFESGQPSSSYGAPKGGFGENRGNRGRPSTSYGVPDSNRNNRGGFGNGGSEARPSTSYGVPGANGNQGGFGSGSIGGRPSTSYGVPGANGNNGDSFRNGDIGGRPSTNYGAPGANGNHGGGNGGNGRPSNNYGVPGANGNTNGKGRLNGNSGGGPSNNYGSPNGFGKGLSTSYGSPNRGGNDNHYPSRGSFINGGINGYSSGSPNGNAGNFGHGDESFGRGGGEGENTGEGYNANAQEESTEPAKYEFSYKVKDQQTGSDYSHTETRDGDHAQGEFNVLLPDGRKQIVEYEADQDGFKPQIRYEGEANADGGYGSGLNDNNDGYSSGRPDSESGGFANSGFNGGSSNGGYPNGGPGERKLGGFNNGGSSGYQSGRSAGQSFGRDNAGDLNNDIGGYFSNSPNNIGDSDNANVGSNRQNDGNSGYQY

SEQ ID NO: 23 - protein, 550 aa, Anopheles darlingi
KREAPLPPSGSYLPPSGGGGGGGGYPAAQTPSSSYGAPAGGAGGWGGNGNGNGNGNGGRGGYSNGGGHSGSAPSQSYGAPSAPSQSYGAPSQSYGAPAAAPSQSYGAPSFGGNGGGASHGSGGFTGGHGGNGNGNGYSSGRPSSQYGPPQQQQQPQQQSFRPPSTSYGVPAAPSSSYGAPSANGFSNGGRPSSQYGAPAPQSNGNEFGAPRPSSSYGAPSRPSTQYGAPSNGNGNGYAGHGNGNGHGNGNGHSNGNGNGYNRGPARQPSSQYGPPSQGPPSSQYGPPSQYGPPSSGTSFIAYGPPSQGPPSSQYGAPAPSRPSSQYGAPAQTPSSQYGAPAQTPSSQYGPPRQSSPQFGAPAPRPPSSQYGAPAQAPSSQYGAPAQTPSSQYGAPAQAPSSQYGAPAPSRPSSQYGVPAQAPSSQYGAPAQAPSSQYGAPAQTPSSQYGAPSFGSTGGSSFGGNGGVGGSYQTASSGNGFSQASFSASSFSSNGRSSQSAGGYSSGGPSQVPATIPQQYSSGGGSYSSGGHSQVPATLPQQYSSNGGYNY

SEQ ID NO: 24 - protein, 585 aa, Acromyrmex echinatior
RSEPPVNSYLPPGPGTSGANGGQTDLSIQYRASDFNNRGNVNGNSGATSFGGPGASNGPSKLYDVPIGGNAGGNGLGQFRGNGFEGGQPSSSYGAPNGGFGENRGNGGKPSTSYGVPDSNGNNRGGFGNGGSEGRPSTSYGLPDASRNNGNGFGNVGNEDKPSTNYGIPANGNKVSGFGNVGSEGRPSTSYGVPGANGNQGFGSGGIGGRPSTSYGVPGVNGNNGGGFENVGRPSTSYGTPDARGNNGGSFRNGDIGGRPSTNYGIPGANGNHGGGNGGNGRPSSNYGVPGGNGNTNGKGRFNGNSGGRPSNSYGSPNGFGKGLSTSYSPSNRDGNGNHYPSGDSNRGSFVNGGINGYPSGSPNGNAGNFRHGDESFGRGGEGGGRSTGEGYNANAQEESTEPAKYEFSYKVKDQQTGSDYSHTETRDGDHAQGEFNVLLPDGRKQIVEYEADQDGFKPQIRYEGEANADGEYDSGGLNDNNDGYSSGRPGSESGGFANNSGFNGGSSNGGYPSGGSGEGKLGFNSGGNSGYQSGRPAGQSFGRDNAGDLSNDIGGFSNSPNNIGGDNANVGSNRQNGGNSGYQY

SEQ ID NO: 25 - protein, 748 aa, Acyrthosiphon pisum
ESPYGGGSSNSNGNGRNGGYGGKGQYGGGNGGGVGSSSASPFFSGANQYGSQSGLSGAANNRYPSFGSKFGGNKGSYGGSSSRNNGRYGSGSASGYGSGSSGGLGSTGRSTGGYGGGSSGSYGSGSSGSLGSSTGSNGIYGAGSSGGFGSGSSGSYGGGSSGGFGSGSSGSYGGGSSGGFGSGSSGSYGGGSSGGFGSGSSGSYGGGSSGGFGSGSSGNYGSGSSGSYGSGGGGLGGASSGNNDGYGAGGSGSYDQLGGANGNGLGGSGNDPLSEPANYEFSYEVNAPESGAIFGHKESRQGEEATGVYHVLLPDGRTQIVEYEADEDGYKPKITYTDPVGGYAGDRQSGNSYGGNGGFGGSGSLGGSGGNLGGLYNGGGSSNNGAGYGGSSSSLGSRYGGSGGSSGSGVGGGYGGSGSSSGGIGSSYGGSGSLSGGLGGGYGGSGSSSGGLGGGYGGSGGSSGGGFGGLGGSGGSSGSGYGGSGSSSGGLGNSYGGSGSSNGGLGGGYSGSGGSSGGLGGGYGASSGSSGSGLGGGYGGSGSSSGGLGSGYGGLGSSSGGLGGGYGGSGSSSGGLGGGYGGSGSSNGGIGGGYGGSSGSSGGLGGGYGGSGSSSGGLGGGYGGSGGSNSGLGSSYGGSGSTNGGLGGGYGGLGSSSGGLGGGYGGSGGSNGGIGGGYGGSSGSGGSQGSAYGGSGSSSGSQGGGYGGSGSSSGGLGGGYGSSSGSSSGLGGSYGSNRNGLGSGSSYS

SEQ ID NO: 26 - protein, 616 aa, Drosophila virilis
RPEPPVNSYLPPSPGDSYGAPGQGQGQGQGGFGGKPSDSYGAPGAGNGNGNGRPSSSYGAPGQGQGQGGFGGKPSDSYGAPGAGNGNGNGRPSSSYGAPGQGQGQGGFGGRPSSSYGAPGQGQGGFGGKPSDTYGAPGAGNGNGRPSSSYGAPGQGQGGIGGKPSDSYGAPGAGNGNGNGRPSSSYGAPGQGQGGFGGKPSDTYGAPGAGNGNGRPSSSYGAPGQGQGGFGGKPSDTYGAPGAGNGNGNGRPSSSYGAPGQGQGGFGGKPSDTYGAPGAGNGNGRPSSSYGAPGQGQGQGGFGGKPSDSYGPPASGAGAGGAGGPGAGGGGDYDNDEPAKYEFNYQVEDAPSGLSFGHSEMRDGDFTTGQYNVLLPDGRKQIVEYEADQQGYRPQVRYEGDANGNGGPGGAGGPGGQDLGQNGYSSGRPGGQDLGQGGYSNGRPGGQDLGQNGYSGGRPGGQDLGQNGYSGGRPGGQDLGQNGYSGGRPGGQDLGQNGYSGGRPGGQDLGQNGYSGGRPGGQDLGQNGYSGGRPGGNGGSDGGRVIIGGRVIGQDGGDGQGYSSGRPNGQDGGFGQDNTDGRGYSSGKPGQGRNGNGNSFGPGGQNGDNDGSGYRY

SEQ ID NO: 27 - protein, 557 aa, Drosophila erecta
RPEPPVNSYLPPSDSYGAPGQSGPGGRPSDSYGAPGGGNGGRPSDSYGAPGLGQGQGQGQGQGGFGGKPSDSYGAPGAGNGNGGRPSSSYGAPGAGNGGRPSDTYGAPGGGSGGRPSDTYGAPGGGNGNGNGGRPSSSYGAPGQGQGNGNSGRPSSSYGAPGAGNGGRPSDTYGAPGGGNGGRPSSSYGAPGAGNGGNGGRPSDTYGAPGGGNGNGNGNGNGSGGRPSSSYGAPGQGQGGFGGRPSDSYGAPGQNQKPSDSYGAPGSGSGSGNGNGGRPSSSYGAPGSGPGGRPSDSYGPPASGSGAGGAGGSGPGGADYDNDIVEYEADQQGYRPQIRYEGDANDGSGPSGPGGQNLGADGYSSGRPGNGNGNGNGGYSGGRPGGQDLGPSGYSGGRPGGQDLGAGGYSNGRPGGQDLGPSGYSGGRPGGQDLGPSGYSGGRPGGQDLGAGGYSNGRPGGNGNGNGGADGGRVIIGGRVIGGQDGGDQGYSGGRPGGQDLGRDGYSSGRPGGRPGANGQDNQDGQGYSSGRSGKGGRNSFGPGGQNGDNDGSGYRY

SEQ ID NO: 28 - protein, 796 aa, Lutzomyia longipalpis
RPEPPANTYLPPSSSYAAPGQQGGSGFGGGGGSGGSGGFGQPGAFGRPSSSYGPPSQGGAGGGFGSDSQFGGGFGGGAGGFGSGGSGAPGASQRPSSSYGPPGQTGGGGFGAQGAPGSSFGPGGGFGGGSPGQAGSPGFQRPSSSYGPPGQSPGGGFSQQGGAPGASQRPSSTYGAPGQGAGGFGQGGSGGFGGTGGSVAIGGRPSSSYGAPGQGSSGGFGGGSGGFGSQAPSTSYGAPGQGSPGGGFGSQGGPGGQPGSPGFGGSQRPSSSYGPPGQGGAPGQGGSPGFGASSRSGGAGGFGASQQPSSSYGPPGQGAGSGFQGTGGGFGGPGQRPGFGGSQTPATSYGAPGQAGGASGGFGGAGAQRPSSSYGPPGQASGFGGGSSGGGFGGGSSGGFGGNQGGFGGNQGGFGGSQTPSSSYGAPSFGSGGSPGAAGGAGGFGQGGVGGSGQPGGFGGGDQGYPPRGGPGGFGPGSGGSGAGGPIAGGSGSGYPGGSDSGSNEPAKYDFSYQVDDPASGTSFGHSEQRDGDYTSGQYNVLLPDGRKQIVEYEADLGGYRPQIKYEGGSSGGAGGYPSGGPGSQGGAGGYPSGGPGGPGSPGGAGGYQSGAAGGAGGYPSGGPGGPGAGGYPSGGPGGPGSQAGGFSGGFGGGSDGAFGGAGGFSQGGAGGGDAGYPRGGPGGFGGAGSPGFGGSGSPGFGGSGSPGAQGSSGFGGTGGGFGGGADGYPRGGPGAGQSGFQDGRGATGGAGQPGGRGSFGRPGSARGGSSSNGYANGGAEGYPRDNPQNRGSGYS

SEQ ID NO: 29 - protein, 1051 aa, Rhodnius prolixus
KRDDPLRRFLAPLVGGGNGSGGGGGGYNYNKPANGLSLPGGGGALPPATSYGVPDRPAPVPSSPPSSSYGAPQPSPNYGAPSSSYGAPSQQPSRSYGAPSQGPSTSYSQRPSSSYGAPAPQTPSSSYGAPAQQPSGSYGAPSGGGGSSGYTGGAQRPSGSYGAPSQGGPSGNYGPPSQQPSSNYGAPSQTPSSNYGAPAQRPSTSYGAPSQPPSSSYGSPPQRASGYPSSSSGPSNGYSPPAQRPSSSYGPPSQQPASSYGAPSQTPSSNYGPPAPIPSSNYGAPSQPPSKPSAPSSSYGTPSQTPSTSYGAPSQAPSSSYGAPSRPSPPSSSYGAPSQGPSSSYGPPSRPSQPSSPSSGYGAPSQGPSSSYGAPSRPSSPSSSYGAPPSSSYGAPSRPSPPSSSYGAPSQGPSSSYGPPSRPSQPSSPSSGYGAPSQGPSSSYGAPSRPSSPSSSYGAPPSSSYGAPSRPSPPSSSYGAPSQGPSSSYGPPSRPSQPSSPSSSYGAPSQGPSSSYGAPSRPSPPSSSYGAPSQGPSSSYGPPSRPSQPSSTYGVPSGGRPSTPSSSYGAPPQALSSTYGAPSGRPGAPSQKPSSSYGAPSLGGNASRGPKSSPPSSSYGAPSVGTSVSSYAPSQGGAGGFQSSRPSSSYGAPSTGPSSTYGPPSQPPSSSYGVPSQPPSSNYGVPSQGVSGSVGSSSPSSSYGAPSQIPSSSYGAPSQSSIGGFGSSRPSSSYGAPPQAPSSSYSAPLRAPSTSYGAPSGGSGSNFGSKPSTNYGAPSQPPSTNYGPPSQPPSSSYGTPSRAPSPTYSTPQSSGTSFGSRPSSSYGVPSQPTTNYGAPSQTPSSNYGAPPASSAPSSTYGRPSQSPSSSYGAPSPSSSSSSYESPSQPPSSSYGAPSQGPSSSYGAPSRPSSTYGAPSPSSPSTNYGAPAPSSNYGTPAQDLTGSYAAPSQPPSAGYGAPSGQPSSGGKQNFQVKNPFAGQTHQVYPAVSSISFGLPSQSFNTAIQGQEPSQSYGAPTASSPSSSYGAPTGTGSSQPGQSYASNGGYSYS

SEQ ID NO: 30 - protein, 506 aa, Rhodnius prolixus
QPPFNHYLPAARGSGSNSAQYTAPSSKFGTSTGQYGQPPSEVPRGLQQGSYAEDVHSSRSVNPSSQNGIPSGHFSSLSSNYGAPSSDYSRSFLRYGTLSNKYGVPNSALGSLSSRNNKTPATQLSYQPSSHYDSRSTSEDQFISSRVSDSQYGASSVRRFLPSSQYSTPSSQYGTPSSQYGTPSSQYGTPSSQYGTPSSQYGTPSSQYGTPSSQYGTPSSQYGTPSSQYGTPSSQYGTPSSPPSQYGGPYSMRTSAPNSQYGTPSSFRTSPSSQFGSSSAHSSSLSKFRSVPSSPYGTLSAIRSTHSSQYGTPSSFSDSTSSSHNGLPSHYPGSGFSGSSVNDQKSYTGNVFGQSHSRVANGDQHARSYTLAGGNEISEPAKYDFNYDVSDGEQGVEFGQEESRDGEETNGSYHVLLPDGRRQRVQYTAGQYGYKPTISYENTGTLTTGRQQFSNGFYNVQQSGSESQEHLGRSTGQNSYGGSNGYESGVGYQSGVGRRSRPAGSY

SEQ ID NO: 31 - protein, 551 aa, Solenopsis invicta
RSEPPINSYLPPRAGSSGANGGRTDLTTQYGAPDFNNGGGATSFSGNGAGDGPSKLYDVPVRGNAGGNGLGRGNGFGGGQPSSSYGAPNGGSNENRGNGGRPSTSYGVPGANGNNGGGFGNGGDKGRPSTSYGVPDASGSSQGSFGNVGNGGRPSTNYGVPGANGNGGGFGNAANEGKPSTSYGVPGANGNSQGGFGNGGRPSTGYGVPGANGNNGGGFGGRPSTSYGAPGANGNHRGGNGGNASPSTNYGVPGGNNGNTNGKGRFNGGNSGGGPSNNYGVPNENAFGGGLSTSYGPPSRGGNGNSGYPSGGSNGGSFVNNGANGYPSGGPNGNAGNFGDGRGGKGGGSSGEGYNDNAQEGSTEPAKYEFSYKVKDQQTGSEYSHTETRDGDRAQGEFNVLLPDGRKQIVEYEADQDGFKPQIRYEGEANAGGGYSSGGSNDNNDGYSSGRPGSEAGGFANNSGFNGSGTNGGRSSGGPGDGNPGGFNSGGGGGYQSGRPAGQSFGRDNDGGLSGDIGGYFANSPSNNIGGSDSANVGSNRQNGGNGGYQY

SEQ ID NO: 32 - protein, 382 aa, Culex quinquefasciatus
KREAPLPGGSYLPPSNGGGAGGYPAAGPPSGSYGPPSNGNGNGNGAGGYPSAPSQQYGAPAGGAPSQQYGAPSNGNGGAGGYPSAPSQQYGAPNGNGNGGFGGRPQAPSQQYGAPSNGNGGARPSQQYGAPNGGNGNGRPQTPSSQYGAPSGGAPSSQYGAPSGGAPSQQYGAPNGGNGNGRPQTPSSQYGAPSGGAPSQQYGAPNGGNGNGRPQTPSSQYGAPSGGAPSSQYGAPSGGAPSSQYGAPAGGAPSSQYGAPAGGAPSSQYGAPAGGAPSSQYGAPAGGAPSSQYGAPAGGAPSSQYGAPSSQYGAPAGGAPSSQYGAPAGGAPSSQYGAPSGGAPSSQYGAPSGGAPSSQYGAPAGGAPSSQYGAPSGGAPSS

SEQ ID NO: 33 - protein, 491 aa, Bactrocera cucurbitae
RPEPPVNSYLPPSANGNGNGGGRPSSQYGAPGLGSNSNGNGNGNGGGRPSSQYGVPGLGGNGNGNGNGGGGGRPSSSYGAPGLGGNGNGNGNGGGRPSSQYGVPGLGGNGNGNGNGNGGGRPSSTYGAPGLRGNGNGNGNGNGRPSSTYGAPGLGGNGNGNGNGNGRPSSTYGAPGLGGNGNGNGNGNGRPSSTYGAPGLNGNGLGGGQKPSDSYGPPASGNGNGYSNGGNGNGNGGGRPGQEYLPPGRNGNGNGNGGRGNGNGGGANGYDYSQGGSDSGESGIVDYEADQGGYRPQIRYEGEANNGAGGLGGGAGGANGYDYEQNGNGLGGGNGYSNGQDLGSNGYSSGRPNGNGNGNGNGNGNGYSGRNGKGRNGNGGGQGLGRNGYSDGRPSGQDLGDNGYASGRPGGNGNGNGGNGNGYSNGNGYSNGNGNGTGNGGGQYNGNGNGYSDGRPGGQDNLDGQGYSSGRPNGFGPGGQNGDNDGNGYRY

SEQ ID NO: 34 - protein, 770 aa, Trichogramma pretiosum
RPEPPVNSYLPPGQGGQGGFGGSGGRPGGGSPSNQYGPPNFQNGGGQNGGSGFGGNGNGNSFGPPSNSYGPPEFGSPGAGSFGGGRPQDTYGPPSNGNGNGNGFGGNGNGGGRPSSRPSDSYGPPSSGNGFGGGNSGRPSESYGPPQNGGGSGNGNQGGGNGFGNGGGRGGQGKPSDSYGPPNSGNRPGSSNGGGQQQNGFGGGNGGRPSNTYGPPGGGNGGGRPGGSSGGFGGQNGGRPSDSYGPPSNGNGNGGRPSNNYGPPNSGGGNGNGFGGSNGKPSNSYGPPSNGNGGGFGGSNGRPSNSYGPPSGGNGGGFGGSSAVGRPGNSGSPSSSGSGFGGNGGASRPSSSYGPPSNGGGFGNGGGSNGRPSSSYGPPNSGSNGGGFGGQNGNGRQNGNNGQGGFGGQPSSSYGPPSNGNGFGGGGGSNGYPQNSQGGNGNGFGQGSGGRPSSSYGPPSNGGGGGDNGYSSGGPGGFGGQPQDSYGPPPSGAVDGNNGFSSGGSSGDNNGYSSGGPGGNGFEDGNDEPAKYEFSYEVKDEQSGSSFGHTEMRDGDRAQGEFNVLLPDGRKQIVEYEADQDGFKPQIRYEGEANTGGAGGYPSGGPGGQGGNGNGGYPSGGPSNGGFGGQNGGGNGGYPSGGPSGGGFGGQNGGSGGYPSGGPSGGGFGGQGGFGGQNSGGNGGYSSGGPASGGFGGQNGGNGGYPSGGPSGGGFGGQGGFGGQNSGGNGGYPSGGPSSGGFGGQNGGGGGNYPAGSGGDAEANGGYQYS

SEQ ID NO: 35 - protein, 280 aa, Drosophila sechellia
QSGAGGRPSDTYGAPGGGNGGRPSDSYGAPGQGQGQGQGQGGYGGKPSDSYGAPGGGNGNGGRPSSSYGAPGGGNGGRPSDTYGAPGGGNGGRPSDTYGAPGGGGNGNGGRPSSSYGAPGQGQGNGNGGRPSSSYGAPGGGNGGRPSDTYGAPGGGNGGRPSDTYGAPGGGNNGGRPSSSYGAPGGGNGGRPSDTYGAPGGGNGNGSGGRPSSSYGAPAQGQGGFGGRPSDSYGAPGQNQKPSDSYGAPGSGNGSAGRPSSSYGAPGSGPGGRPSDSYGP

SEQ ID NO: 36 - protein, 280 aa, Drosophila sechellia; identical to SEQ ID NO: 35

SEQ ID NO: 37 - protein, 280 aa, Drosophila sechellia; identical to SEQ ID NO: 35

SEQ ID NO: 38 - protein, 280 aa, Drosophila sechellia; identical to SEQ ID NO: 35

SEQ ID NO: 39 - protein, 162 aa, Drosophila sechellia
YSSGRPGNGNGNGNGGYSSGRPGGQDLGPSGYSGGRPGGQDLGAGGYSNVKPGGQDLGPGGYSGGRPGGQDLGRDGYSGGRPGGQDLGAGAYSNGRPGGNGNGGSDGGRVIIGGRVIGGQDGGDQGYSGGRPGGQDLGRDGYSSGRPGGRPGGNGQDSQDGQ

SEQ ID NO: 40 - protein, 162 aa, Drosophila sechellia; identical to SEQ ID NO: 39

SEQ ID NO: 41 - protein, 162 aa, Drosophila sechellia; identical to SEQ ID NO: 39

SEQ ID NO: 42 - protein, 162 aa, Drosophila sechellia; identical to SEQ ID NO: 39

SEQ ID NO: 43 - protein, 162 aa, Drosophila sechellia; identical to SEQ ID NO: 39

SEQ ID NO: 44 - protein, 162 aa, Drosophila sechellia; identical to SEQ ID NO: 39

SEQ ID NO: 45 - protein, 25 aa, synthetic construct
  REGION 1..25: note = Description of Artificial Sequence: Synthetic peptide
GDYKDDDDKDYKDDDDKDYKDDDDK

SEQ ID NO: 46 - protein, 85 aa, unidentified
  REGION 1..85: note = Description of Unknown: Alpha mating factor precursor protein sequence
MRFPSIFTAVLFAASSALAAPVNTTTEDETAQIPAEAVIGYSDLEGDFDVAVLPFSNSTNNGLLFINTTIASIAAKEEGVSLEKR

SEQ ID NO: 47 - protein, 4 aa, synthetic construct
  REGION 1..4: note = Description of Artificial Sequence: Synthetic peptide
EAEA

SEQ ID NO: 48 - skipped

SEQ ID NO: 49 - protein, 722 aa, synthetic construct
  REGION 1..722: note = Description of Artificial Sequence: Synthetic polypeptide
MRFPSIFTAVLFAASSALAAPVNTTTEDETAQIPAEAVIGYSDLEGDFDVAVLPFSNSTNNGLLFINTTIASIAAKEEGVSLEKREAEAGRPEPPVNSYLPPSDSYGAPGQSGAGGRPSDTYGAPGGGNGGRPSDSYGAPGQGQGQGQGQGGYGGKPSDSYGAPGGGNGNGGRPSSSYGAPGGGNGGRPSDTYGAPGGGNGGRPSDTYGAPGGGGNGNGGRPSSSYGAPGQGQGNGNGGRPSSSYGAPGGGNGGRPSDTYGAPGGGNGGRPSDTYGAPGGGNNGGRPSSSYGAPGGGNGGRPSDTYGAPGGGNGNGSGGRPSSSYGAPAQGQGGFGGRPSDSYGAPGQNQKPSDSYGAPGSGNGSAGRPSSSYGAPGSGPGGRPSDSYGPPASGSGAGGAGGSGPGGADYDNDEPAKYEFNYQVEDAPSGLSFGHSEMRDGDFTTGQYNVLLPDGRKQIVEYEADQQGYRPQIRYEGDANDGSGPSGPSGPGGPGGQNLGADGYSSGRPGNGNGNGNGGYSSGRPGGQDLGPSGYSGGRPGGQDLGAGGYSNVKPGGQDLGPGGYSGGRPGGQDLGRDGYSGGRPGGQDLGAGAYSNGRPGGNGNGGSDGGRVIIGGRVIGGQDGGDQGYSGGRPGGQDLGRDGYSSGRPGGRPGGNGQDSQDGQGYSSGRPGQGGRNGFGPGGQNGDNDGSGYRYSGDYKDDDDKDYKDDDDKDYKDDDDK

SEQ ID NO: 50 - protein, 7 aa, synthetic construct
  REGION 1..7: note = Description of Artificial Sequence: Synthetic peptide
  VARIANT 2..3: note = Any amino acid
  VARIANT 6: note = Any amino acid
SXXYGXP

SEQ ID NO: 51 - protein, 4 aa, synthetic construct
  REGION 1..4: note = Description of Artificial Sequence: Synthetic peptide
GGGG

SEQ ID NO: 52 - protein, 4 aa, synthetic construct
  REGION 1..4: note = Description of Artificial Sequence: Synthetic peptide
NGNG

SEQ ID NO: 53 - protein, 4 aa, synthetic construct
  REGION 1..4: note = Description of Artificial Sequence: Synthetic peptide
GQGG

SEQ ID NO: 54 - protein, 4 aa, synthetic construct
  REGION 1..4: note = Description of Artificial Sequence: Synthetic peptide
GQGN

SEQ ID NO: 55 - protein, 4 aa, synthetic construct
  REGION 1..4: note = Description of Artificial Sequence: Synthetic peptide
GQGQ

SEQ ID NO: 56 - protein, 5 aa, synthetic construct
  REGION 1..5: note = Description of Artificial Sequence: Synthetic peptide
GQGQG

SEQ ID NO: 57 - protein, 5 aa, synthetic construct
  REGION 1..5: note = Description of Artificial Sequence: Synthetic peptide
RPGGQ

SEQ ID NO: 58 - protein, 5 aa, synthetic construct
  REGION 1..5: note = Description of Artificial Sequence: Synthetic peptide
RPGGN

SEQ ID NO: 59 - protein, 4 aa, synthetic construct
  REGION 1..4: note = Description of Artificial Sequence: Synthetic peptide
GGSF

SEQ ID NO: 60 - protein, 4 aa, synthetic construct
  REGION 1..4: note = Description of Artificial Sequence: Synthetic peptide
GNGG

SEQ ID NO: 61 - protein, 5 aa, synthetic construct
  REGION 1..5: note = Description of Artificial Sequence: Synthetic peptide
GGAGG

SEQ ID NO: 62 - protein, 12 aa, synthetic construct
  REGION 1..12: note = Description of Artificial Sequence: Synthetic peptide
  VARIANT 1..2: note = Any amino acid
XXEPPVSYLPPS

SEQ ID NO: 63 - protein, 4 aa, unidentified
  REGION 1..4: note = Description of Unknown: "GRPE" sequence
GRPE

SEQ ID NO: 64 - protein, 3000 aa, synthetic construct
  REGION 1..3000: note = Description of Artificial Sequence: Synthetic polypeptide
  VARIANT 1..3000: note = MISC_FEATURE - This sequence may encompass 1-100 "Tyr Gly Xaa Pro Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa" repeating units
  VARIANT 3: note = A, L, P, T or V
  VARIANT 5..24: note = Any amino acid
  VARIANT 5..24: note = MISC_FEATURE - This region may encompass 3-20 residues
  VARIANT 25..28: note = This region may encompass one of the following sequences: GG, LS, APS, GAG, GGG, KPS, RPS, or GGGG
  VARIANT 29..30: note = This region may encompass one of the following sequences: S, D, T, N, L, DS, DT, LS, SS, ST, TN, or TS
  VARIANT 33: note = A, L, P, T or V
  VARIANT 35..54: note = Any amino acid
  VARIANT 35..54: note = MISC_FEATURE - This region may encompass 3-20 residues
  VARIANT 55..58: note = This region may encompass one of the following sequences: GG, LS, APS, GAG, GGG, KPS, RPS, or GGGG
  VARIANT 59..60: note = This region may encompass one of the following sequences: S, D, T, N, L, DS, DT, LS, SS, ST, TN, or TS
  VARIANT 63: note = A, L, P, T or V
  VARIANT 65..84: note = Any amino acid
  VARIANT 65..84: note = MISC_FEATURE - This region may encompass 3-20 residues
  VARIANT 85..88: note = This region may encompass one of the following sequences: GG, LS, APS, GAG, GGG, KPS, RPS, or GGGG
  VARIANT 89..90: note = This region may encompass one of the following sequences: S, D, T, N, L, DS, DT, LS, SS, ST, TN, or TS
  VARIANT 93: note = A, L, P, T or V
  VARIANT 95..114: note = Any amino acid
  VARIANT 95..114: note = MISC_FEATURE - This region may encompass 3-20 residues
  VARIANT 115..118: note = This region may encompass one of the following sequences: GG, LS, APS, GAG, GGG, KPS, RPS, or GGGG
  VARIANT 119..120: note = This region may encompass one of the following sequences: S, D, T, N, L, DS, DT, LS, SS, ST, TN, or TS
  VARIANT 123: note = A, L, P, T or V
  VARIANT 125..144: note = Any amino acid
  VARIANT 125..144: note = MISC_FEATURE - This region may encompass 3-20 residues
  VARIANT 145..148: note = This region may encompass one of the following sequences: GG, LS, APS, GAG, GGG, KPS, RPS, or GGGG
  VARIANT 149..150: note = This region may encompass one of the following sequences: S, D, T, N, L, DS, DT, LS, SS, ST, TN, or TS
  VARIANT 153: note = A, L, P, T or V
  VARIANT 155..174: note = Any amino acid
  VARIANT 155..174: note = MISC_FEATURE - This region may encompass 3-20 residues
  VARIANT 175..178: note = This region may encompass one of the following sequences: GG, LS, APS, GAG, GGG, KPS, RPS, or GGGG
  VARIANT 179..180: note = This region may encompass one of the following sequences: S, D, T, N, L, DS, DT, LS, SS, ST, TN, or TS
  VARIANT 183: note = A, L, P, T or V
  VARIANT 185..204: note = Any amino acid
  VARIANT 185..204: note = MISC_FEATURE - This region may encompass 3-20 residues
  VARIANT 205..208: note = This region may encompass one of the following sequences: GG, LS, APS, GAG, GGG, KPS, RPS, or GGGG
  VARIANT 209..210: note = This region may encompass one of the following sequences: S, D, T, N, L, DS, DT, LS, SS, ST, TN, or TS
  VARIANT 213: note = A, L, P, T or V
  VARIANT 215..234: note = Any amino acid
  VARIANT 215..234: note = MISC_FEATURE - This region may encompass 3-20 residues
  VARIANT 235..238: note = This region may encompass one of the following sequences: GG, LS, APS, GAG, GGG, KPS, RPS, or GGGG
  VARIANT 239..240: note = This region may encompass one of the following sequences: S, D, T, N, L, DS, DT, LS, SS, ST, TN, or TS
  VARIANT 243: note = A, L, P, T or V
  VARIANT 245..264: note = Any amino acid
  VARIANT 245..264: note = MISC_FEATURE - This region may encompass 3-20 residues
  VARIANT 265..268: note = This region may encompass one of the following sequences: GG, LS, APS, GAG, GGG, KPS, RPS, or GGGG
  VARIANT 269..270: note = This region may encompass one of the following sequences: S, D, T, N, L, DS, DT, LS, SS, ST, TN, or TS
  VARIANT 273: note = A, L, P, T or V
  VARIANT 275..294: note = Any amino acid
  VARIANT 275..294: note = MISC_FEATURE - This region may encompass 3-20 residues
  VARIANT 295..298: note = This region may encompass one of the following sequences: GG, LS, APS, GAG, GGG, KPS, RPS, or GGGG
  VARIANT 299..300: note = This region may encompass one of the following sequences: S, D, T, N, L, DS, DT, LS, SS, ST, TN, or TS
  VARIANT 303: note = A, L, P, T or V
  VARIANT 305..324: note = Any amino acid
  VARIANT 305..324: note = MISC_FEATURE - This region may encompass 3-20 residues
  VARIANT 325..328: note = This region may encompass one of the following sequences: GG, LS, APS, GAG, GGG, KPS, RPS, or GGGG
  VARIANT 329..330: note = This region may encompass one of the following sequences: S, D, T, N, L, DS, DT, LS, SS, ST, TN, or TS
  VARIANT 333: note = A, L, P, T or V
  VARIANT 335..354: note = Any amino acid
  VARIANT 335..354: note = MISC_FEATURE - This region may encompass 3-20 residues
  VARIANT 355..358: note = This region may encompass one of the following sequences: GG, LS, APS, GAG, GGG, KPS, RPS, or GGGG
  VARIANT 359..360: note = This region may encompass one of the following sequences: S, D, T, N, L, DS, DT, LS, SS, ST, TN, or TS
  VARIANT 363: note = A, L, P, T or V
  VARIANT 365..384: note = Any amino acid
  VARIANT 365..384: note = MISC_FEATURE - This region may encompass 3-20 residues
  VARIANT 385..388: note = This region may encompass one of the following sequences: GG, LS, APS, GAG, GGG, KPS, RPS, or GGGG
  VARIANT 389..390: note = This region may encompass one of the following sequences: S, D, T, N, L, DS, DT, LS, SS, ST, TN, or TS
  VARIANT 393: note = A, L, P, T or V
  VARIANT 395..414: note = Any amino acid
  VARIANT 395..414: note = MISC_FEATURE - This region may encompass 3-20 residues
  VARIANT 415..418: note = This region may encompass one of the following sequences: GG, LS, APS, GAG, GGG, KPS, RPS, or GGGG
  VARIANT 419..420: note = This region may encompass one of the following sequences: S, D, T, N, L, DS, DT, LS, SS, ST, TN, or TS
  VARIANT 423: note = A, L, P, T or V
  VARIANT 425..444: note = Any amino acid
  VARIANT 425..444: note = MISC_FEATURE - This region may encompass 3-20 residues
  VARIANT 445..448: note = This region may encompass one of the following sequences: GG, LS, APS, GAG, GGG, KPS, RPS, or GGGG
  VARIANT 449..450: note = This region may encompass one of the following sequences: S, D, T, N, L, DS, DT, LS, SS, ST, TN, or TS
  VARIANT 453: note = A, L, P, T or V
  VARIANT 455..474: note = Any amino acid
  VARIANT 455..474: note = MISC_FEATURE - This region may encompass 3-20 residues
  VARIANT 475..478: note = This region may encompass one of the following sequences: GG, LS, APS, GAG, GGG, KPS, RPS, or GGGG
  VARIANT 479..480: note = This region may encompass one of the following sequences: S, D, T, N, L, DS, DT, LS, SS, ST, TN, or TS
  VARIANT 483: note = A, L, P, T or V
  VARIANT 485..504: note = Any amino acid
  VARIANT 485..504: note = MISC_FEATURE - This region may encompass 3-20 residues
  VARIANT 505..508: note = This region may encompass one of the following sequences: GG, LS, APS, GAG, GGG, KPS, RPS, or GGGG
  VARIANT 509..510: note = This region may encompass one of the following sequences: S, D, T, N, L, DS, DT, LS, SS, ST, TN, or TS
  VARIANT 513: note = A, L, P, T or V
  VARIANT 515..534: note = Any amino acid
  VARIANT 515..534: note = MISC_FEATURE - This region may encompass 3-20 residues
  VARIANT 535..538: note = This region may encompass one of the following sequences: GG, LS, APS, GAG, GGG, KPS, RPS, or GGGG
  VARIANT 539..540: note = This region may encompass one of the following sequences: S, D, T, N, L, DS, DT, LS, SS, ST, TN, or TS
  VARIANT 543: note = A, L, P, T or V
  VARIANT 545..564: note = Any amino acid
  VARIANT 545..564: note = MISC_FEATURE - This region may encompass 3-20 residues
  VARIANT 565..568: note = This region may encompass one of the following sequences: GG, LS, APS, GAG, GGG, KPS, RPS, or GGGG
  VARIANT 569..570: note = This region may encompass one of the following sequences: S, D, T, N, L, DS, DT, LS, SS, ST, TN, or TS
  VARIANT 573: note = A, L, P, T or V
  VARIANT 575..594: note = Any amino acid
  VARIANT 575..594: note = MISC_FEATURE - This region may encompass 3-20 residues
  VARIANT 595..598: note = This region may encompass one of the following sequences: GG, LS, APS, GAG, GGG, KPS, RPS, or GGGG
  VARIANT 599..600: note = This region may encompass one of the following sequences: S, D, T, N, L, DS, DT, LS, SS, ST, TN, or TS
  VARIANT 603: note = A, L, P, T or V
  VARIANT 605..624: note = Any amino acid
  VARIANT 605..624: note = MISC_FEATURE - This region may encompass 3-20 residues
  VARIANT 625..628: note = This region may encompass one of the following sequences: GG, LS, APS, GAG, GGG, KPS, RPS, or GGGG
  VARIANT 629..630: note = This region may encompass one of the following sequences: S, D, T, N, L, DS, DT, LS, SS, ST, TN, or TS
  VARIANT 633: note = A, L, P, T or V
  VARIANT 635..654: note = Any amino acid
  VARIANT 635..654: note = MISC_FEATURE - This region may encompass 3-20 residues
  VARIANT 655..658: note = This region may encompass one of the following sequences: GG, LS, APS, GAG, GGG, KPS, RPS, or GGGG
  VARIANT 659..660: note = This region may encompass one of the following sequences: S, D, T, N, L, DS, DT, LS, SS, ST, TN, or TS
  VARIANT 663: note = A, L, P, T or V
  VARIANT 665..684: note = Any amino acid
  VARIANT 665..684: note = MISC_FEATURE - This region may encompass 3-20 residues
  VARIANT 685..688: note = This region may encompass one of the following sequences: GG, LS, APS, GAG, GGG, KPS, RPS, or GGGG
  VARIANT 689..690: note = This region may encompass one of the following sequences: S, D, T, N, L, DS, DT, LS, SS, ST, TN, or TS
  VARIANT 693: note = A, L, P, T or V
  VARIANT 695..714: note = Any amino acid
  VARIANT 695..714: note = MISC_FEATURE - This region may encompass 3-20 residues
  VARIANT 715..718: note = This region may encompass one of the following sequences: GG, LS, APS, GAG, GGG, KPS, RPS, or GGGG
  VARIANT 719..720: note = This region may encompass one of the following sequences: S, D, T, N, L, DS, DT, LS, SS, ST, TN, or TS
  VARIANT 723: note = A, L, P, T or V
  VARIANT 725..744: note = Any amino acid
  VARIANT 725..744: note = MISC_FEATURE - This region may encompass 3-20 residues
  VARIANT 745..748: note = This region may encompass one of the following sequences: GG, LS, APS, GAG, GGG, KPS, RPS, or GGGG
  VARIANT 749..750: note = This region may encompass one of the following sequences: S, D, T, N, L, DS, DT, LS, SS, ST, TN, or TS
  VARIANT 753: note = A, L, P, T or V
  VARIANT 755..774: note = Any amino acid
  VARIANT 755..774: note = MISC_FEATURE - This region may encompass 3-20 residues
  VARIANT 775..778: note = This region may encompass one of the following sequences: GG, LS, APS, GAG, GGG, KPS, RPS, or GGGG
  VARIANT 779..780: note = This region may encompass one of the following sequences: S, D, T, N, L, DS, DT, LS, SS, ST, TN, or TS
  VARIANT 783: note = A, L, P, T or V
  VARIANT 785..804: note = Any amino acid
  VARIANT 785..804: note = MISC_FEATURE - This region may encompass 3-20 residues
  VARIANT 805..808: note = This region may encompass one of the following sequences: GG, LS, APS, GAG, GGG, KPS, RPS, or GGGG
  VARIANT 809..810: note = This region may encompass one of the following sequences: S, D, T, N, L, DS, DT, LS, SS, ST, TN, or TS
  VARIANT 813: note = A, L, P, T or V
  VARIANT 815..834: note = Any amino acid
  VARIANT 815..834: note = MISC_FEATURE - This region may encompass 3-20 residues
  VARIANT 835..838: note = This region may encompass one of the following sequences: GG, LS, APS, GAG, GGG, KPS, RPS, or GGGG
  VARIANT 839..840: note = This region may encompass one of the following sequences: S, D, T, N, L, DS, DT, LS, SS, ST, TN, or TS
  VARIANT 843: note = A, L, P, T or V
  VARIANT 845..864: note = Any amino acid
  VARIANT 845..864: note = MISC_FEATURE - This region may encompass 3-20 residues
  VARIANT 865..868: note = This region may encompass one of the following sequences: GG, LS, APS, GAG, GGG, KPS, RPS, or GGGG
  VARIANT 869..870: note = This region may encompass one of the following sequences: S, D, T, N, L, DS, DT, LS, SS, ST, TN, or TS
  VARIANT 873: note = A, L, P, T or V
  VARIANT 875..894: note = Any amino acid
  VARIANT 875..894: note = MISC_FEATURE - This region may encompass 3-20 residues
  VARIANT 895..898: note = This region may encompass one of the following sequences: GG, LS, APS, GAG, GGG, KPS, RPS, or GGGG
  VARIANT 899..900: note = This region may encompass one of the following sequences: S, D, T, N, L, DS, DT, LS, SS, ST, TN, or TS
  VARIANT 903: note = A, L, P, T or V
  VARIANT 905..924: note = Any amino acid
  VARIANT 905..924: note = MISC_FEATURE - This region may encompass 3-20 residues
  VARIANT 925..928: note = This region may encompass one of the following sequences: GG, LS, APS, GAG, GGG, KPS, RPS, or GGGG
  VARIANT 929..930: note = This region may encompass one of the following sequences: S, D, T, N, L, DS, DT, LS, SS, ST, TN, or TS
  VARIANT 933: note = A, L, P, T or V
  VARIANT 935..954: note = Any amino acid
  VARIANT 935..954: note = MISC_FEATURE - This region may encompass 3-20 residues
  VARIANT 955..958: note = This region may encompass one of the following sequences: GG, LS, APS, GAG, GGG, KPS, RPS, or GGGG
  VARIANT 959..960: note = This region may encompass one of the following sequences: S, D, T, N, L, DS, DT, LS, SS, ST, TN, or TS
  VARIANT 963: note = A, L, P, T or V
  VARIANT 965..984: note = Any amino acid
  VARIANT 965..984: note = MISC_FEATURE - This region may encompass 3-20 residues
  VARIANT 985..988: note = This region may encompass one of the following sequences: GG, LS, APS, GAG, GGG, KPS, RPS, or GGGG
  VARIANT 989..990: note = This region may encompass one of the following sequences: S, D, T, N, L, DS, DT, LS, SS, ST, TN, or TS
  VARIANT 993: note = A, L, P, T or V
  VARIANT 995..1014: note = Any amino acid
  VARIANT 995..1014: note = MISC_FEATURE - This region may encompass 3-20 residues
  VARIANT 1015..1018: note = This region may encompass one of the following sequences: GG, LS, APS, GAG, GGG, KPS, RPS, or GGGG
  VARIANT 1019..1020: note = This region may encompass one of the following sequences: S, D, T, N, L, DS, DT, LS, SS, ST, TN, or TS
  VARIANT 1023: note = A, L, P, T or V
  VARIANT 1025..1044: note = Any amino acid
  VARIANT 1025..1044: note = MISC_FEATURE - This region may encompass 3-20 residues
  VARIANT 1045..1048: note = This region may encompass one of the following sequences: GG, LS, APS, GAG, GGG, KPS, RPS, or GGGG
  VARIANT 1049..1050: note = This region may encompass one of the following sequences: S, D, T, N, L, DS, DT, LS, SS, ST, TN, or TS
  VARIANT 1053: note = A, L, P, T or V
  VARIANT 1055..1074: note = Any amino acid
  VARIANT 1055..1074: note = MISC_FEATURE - This region may encompass 3-20 residues
  VARIANT 1075..1078: note = This region may encompass one of the following sequences: GG, LS, APS, GAG, GGG, KPS, RPS, or GGGG
  VARIANT 1079..1080: note = This region may encompass one of the following sequences: S, D, T, N, L, DS, DT, LS, SS, ST, TN, or TS
  VARIANT 1083: note = A, L, P, T or V
  VARIANT 1085..1104: note = Any amino acid
  VARIANT 1085..1104: note = MISC_FEATURE - This region may encompass 3-20 residues
  VARIANT 1105..1108: note = This region may encompass one of the following sequences: GG, LS, APS, GAG, GGG, KPS, RPS, or GGGG
  VARIANT 1109..1110: note = This region may encompass one of the following sequences: S, D, T, N, L, DS, DT, LS, SS, ST, TN, or TS
  VARIANT 1113: note = A, L, P, T or V
  VARIANT 1115..1134: note = Any amino acid
  VARIANT 1115..1134: note = MISC_FEATURE - This region may encompass 3-20 residues
  VARIANT 1135..1138: note = This region may encompass one of the following sequences: GG, LS, APS, GAG, GGG, KPS, RPS, or GGGG
  VARIANT 1139..1140: note = This region may encompass one of the following sequences: S, D, T, N, L, DS, DT, LS, SS, ST, TN, or TS
  VARIANT 1143: note = A, L, P, T or V
  VARIANT 1145..1164: note = Any amino acid
  VARIANT 1145..1164: note = MISC_FEATURE - This region may encompass 3-20 residues
  VARIANT 1165..1168: note = This region may encompass one of the following sequences: GG, LS, APS, GAG, GGG, KPS, RPS, or GGGG
  VARIANT 1169..1170: note = This region may encompass one of the following sequences: S, D, T, N, L, DS, DT, LS, SS, ST, TN, or TS
  VARIANT 1173: note = A, L, P, T or V
  VARIANT 1175..1194: note = Any amino acid
  VARIANT 1175..1194: note = MISC_FEATURE - This region may encompass 3-20 residues
  VARIANT 1195..1198: note = This region may encompass one of the following sequences: GG, LS, APS, GAG, GGG, KPS, RPS, or GGGG
  VARIANT 1199..1200: note = This region may encompass one of the following sequences: S, D, T, N, L, DS, DT, LS, SS, ST, TN, or TS
  VARIANT 1203: note = A, L, P, T or V
  VARIANT 1205..1224: note = Any amino acid
  VARIANT 1205..1224: note = MISC_FEATURE - This region may encompass 3-20 residues
  VARIANT 1225..1228: note = This region may encompass one of the following sequences: GG, LS, APS, GAG, GGG, KPS, RPS, or GGGG
  VARIANT 1229..1230: note = This region may encompass one of the following sequences: S, D, T, N, L, DS, DT, LS, SS, ST, TN, or TS
  VARIANT 1233: note = A, L, P, T or V
  VARIANT 1235..1254: note = Any amino acid
  VARIANT 1235..1254: note = MISC_FEATURE - This region may encompass 3-20 residues
  VARIANT 1255..1258: note = This region may encompass one of the following sequences: GG, LS, APS, GAG, GGG, KPS, RPS, or GGGG
  VARIANT 1259..1260: note = This region may encompass one of the following sequences: S, D, T, N, L, DS, DT, LS, SS, ST, TN, or TS
  VARIANT 1263: note = A, L, P, T or V
  VARIANT 1265..1284: note = Any amino acid
  VARIANT 1265..1284: note = MISC_FEATURE - This region may encompass 3-20 residues
  VARIANT 1285..1288: note = This region may encompass one of the following sequences: GG, LS, APS, GAG, GGG, KPS, RPS, or GGGG
  VARIANT 1289..1290: note = This region may encompass one of the following sequences: S, D, T, N, L, DS, DT, LS, SS, ST, TN, or TS
  VARIANT 1293: note = A, L, P, T or V
  VARIANT 1295..1314: note = Any amino acid
  VARIANT 1295..1314: note = MISC_FEATURE - This region may encompass 3-20 residues
  VARIANT 1315..1318: note = This region may encompass one of the following sequences: GG, LS, APS, GAG, GGG, KPS, RPS, or GGGG
  VARIANT 1319..1320: note = This region may encompass one of the following sequences: S, D, T, N, L, DS, DT, LS, SS, ST, TN, or TS
  VARIANT 1323: note = A, L, P, T or V
  VARIANT 1325..1344: note = Any amino acid
  VARIANT 1325..1344: note = MISC_FEATURE - This region may encompass 3-20 residues
  VARIANT 1345..1348: note = This region may encompass one of the following sequences: GG, LS, APS, GAG, GGG, KPS, RPS, or GGGG
  VARIANT 1349..1350: note = This region may encompass one of the following sequences: S, D, T, N, L, DS, DT, LS, SS, ST, TN, or TS
  VARIANT 1353: note = A, L, P, T or V
  VARIANT 1355..1374: note = Any amino acid
  VARIANT 1355..1374: note = MISC_FEATURE - This region may encompass 3-20 residues
  VARIANT 1375..1378: note = This region may encompass one of the following sequences: GG, LS, APS, GAG, GGG, KPS, RPS, or GGGG
  VARIANT 1379..1380: note = This region may encompass one of the following sequences: S, D, T, N, L, DS, DT, LS, SS, ST, TN, or TS
  VARIANT 1383: note = A, L, P, T or V
  VARIANT 1385..1404: note = Any amino acid
  VARIANT 1385..1404: note = MISC_FEATURE - This region may encompass 3-20 residues
  VARIANT 1405..1408: note = This region may encompass one of the following sequences: GG, LS, APS, GAG, GGG, KPS, RPS, or GGGG
  VARIANT 1409..1410: note = This region may encompass one of the following sequences: S, D, T, N, L, DS, DT, LS, SS, ST, TN, or TS
  VARIANT 1413: note = A, L, P, T or V
  VARIANT 1415..1434: note = Any amino acid
  VARIANT 1415..1434: note = MISC_FEATURE - This region may encompass 3-20 residues
  VARIANT 1435..1438: note = This region may encompass one of the following sequences: GG, LS, APS, GAG, GGG, KPS, RPS, or GGGG
  VARIANT 1439..1440: note = This region may encompass one of the following sequences: S, D, T, N, L, DS, DT, LS, SS, ST, TN, or TS
  VARIANT 1443: note = A, L, P, T or V
  VARIANT 1445..1464: note = Any amino acid
  VARIANT 1445..1464: note = MISC_FEATURE - This region may encompass 3-20 residues
  VARIANT 1465..1468: note = This region may encompass one of the following sequences: GG, LS, APS, GAG, GGG, KPS, RPS, or GGGG
  VARIANT 1469..1470: note = This region may encompass one of the following sequences: S, D, T, N, L, DS, DT, LS, SS, ST, TN, or TS
  VARIANT 1473: note = A, L, P, T or V
  VARIANT 1475..1494: note = Any amino acid
  VARIANT 1475..1494: note = MISC_FEATURE - This region may encompass 3-20 residues
  VARIANT 1495..1498: note = This region may encompass one of the following sequences: GG, LS, APS, GAG, GGG, KPS, RPS, or GGGG
  VARIANT 1499..1500: note = This region may encompass one of the following sequences: S, D, T, N, L, DS, DT, LS, SS, ST, TN, or TS
  VARIANT 1503: note = A, L, P, T or V
  VARIANT 1505..1524: note = Any amino acid
  VARIANT 1505..1524: note = MISC_FEATURE - This region may encompass 3-20 residues
  VARIANT 1525..1528: note = This region may encompass one of the following sequences: GG, LS, APS, GAG, GGG, KPS, RPS, or GGGG
  VARIANT 1529..1530: note = This region may encompass one of the following sequences: S, D, T, N, L, DS, DT, LS, SS, ST, TN, or TS
  VARIANT 1533: note = A, L, P, T or V
  VARIANT 1535..1554: note = Any amino acid
  VARIANT 1535..1554: note = MISC_FEATURE - This region may encompass 3-20 residues
  VARIANT 1555..1558: note = This region may encompass one of the following sequences: GG, LS, APS, GAG, GGG, KPS, RPS, or GGGG
  VARIANT 1559..1560: note = This region may encompass one of the following sequences: S, D, T, N, L, DS, DT, LS, SS, ST, TN, or TS
  VARIANT 1563: note = A, L, P, T or V
  VARIANT 1565..1584: note = Any amino acid
  VARIANT 1565..1584: note = MISC_FEATURE - This region may encompass 3-20 residues
  VARIANT 1585..1588: note = This region may encompass one of the following sequences: GG, LS, APS, GAG, GGG, KPS, RPS, or GGGG
  VARIANT 1589..1590: note = This region may encompass one of the following sequences: S, D, T, N, L, DS, DT, LS, SS, ST, TN, or TS
  VARIANT 1593: note = A, L, P, T or V
  VARIANT 1595..1614: note = Any amino acid
  VARIANT 1595..1614: note = MISC_FEATURE - This region may encompass 3-20 residues
  VARIANT 1615..1618: note = This region may encompass one of the following sequences: GG, LS, APS, GAG, GGG, KPS, RPS, or GGGG
  VARIANT 1619..1620: note = This region may encompass one of the following sequences: S, D, T, N, L, DS, DT, LS, SS, ST, TN, or TS
  VARIANT 1623: note = A, L, P, T or V
  VARIANT 1625..1644: note = Any amino acid
  VARIANT 1625..1644: note = MISC_FEATURE - This region may encompass 3-20 residues
  VARIANT 1645..1648: note = This region may encompass one of the following sequences: GG, LS, APS, GAG, GGG, KPS, RPS, or GGGG
  VARIANT 1649..1650: note = This region may encompass one of the following sequences: S, D, T, N, L, DS, DT, LS, SS, ST, TN, or TS
  VARIANT 1653: note = A, L, P, T or V
  VARIANT 1655..1674: note = Any amino acid
  VARIANT 1655..1674: note = MISC_FEATURE - This region may encompass 3-20 residues
  VARIANT 1675..1678: note = This region may encompass one of the following sequences: GG, LS, APS, GAG, GGG, KPS, RPS, or GGGG
  VARIANT 1679..1680: note = This region may encompass one of the following sequences: S, D, T, N, L, DS, DT, LS, SS, ST, TN, or TS
  VARIANT 1683: note = A, L, P, T or V
  VARIANT 1685..1704: note = Any amino acid
  VARIANT 1685..1704: note = MISC_FEATURE - This region may encompass 3-20 residues
  VARIANT 1705..1708: note = This region may encompass one of the following sequences: GG, LS, APS, GAG, GGG, KPS, RPS, or GGGG
  VARIANT 1709..1710: note = This region may encompass one of the following sequences: S, D, T, N, L, DS, DT, LS, SS, ST, TN, or TS
  VARIANT 1713: note = A, L, P, T or V
  VARIANT 1715..1734: note = Any amino acid
  VARIANT 1715..1734: note = MISC_FEATURE - This region may encompass 3-20 residues
  VARIANT 1735..1738: note = This region may encompass one of the following sequences: GG, LS, APS, GAG, GGG, KPS, RPS, or GGGG
  VARIANT 1739..1740: note = This region may encompass one of the following sequences: S, D, T, N, L, DS, DT, LS, SS, ST, TN, or TS
  VARIANT 1743: note = A, L, P, T or V
  VARIANT 1745..1764: note = Any amino acid
  VARIANT 1745..1764: note = MISC_FEATURE - This region may encompass 3-20 residues
  VARIANT 1765..1768: note = This region may encompass one of the following sequences: GG, LS, APS, GAG, GGG, KPS, RPS, or GGGG
  VARIANT 1769..1770: note = This region may encompass one of the following sequences: S, D, T, N, L, DS, DT, LS, SS, ST, TN, or TS
  VARIANT 1773: note = A, L, P, T or V
  VARIANT 1775..1794: note = Any amino acid
  VARIANT 1775..1794: note = MISC_FEATURE - This region may encompass 3-20 residues
  VARIANT 1795..1798: note = Any amino acid
  VARIANT 1799..1800: note = This region may encompass one of the following sequences: S, D, T, N, L, DS, DT, LS, SS, ST, TN, or TS
  VARIANT 1803: note = A, L, P, T or V
  VARIANT 1805..1824: note = Any amino acid
  VARIANT 1805..1824: note = MISC_FEATURE - This region may encompass 3-20 residues
  VARIANT 1825..1828: note = This region may encompass one of the following sequences: GG, LS, APS, GAG, GGG, KPS, RPS, or GGGG
  VARIANT 1829..1830: note = This region may encompass one of the following sequences: S, D, T, N, L, DS, DT, LS, SS, ST, TN, or TS
  VARIANT 1833: note = A, L, P, T or V
  VARIANT 1835..1854: note = Any amino acid
  VARIANT 1835..1854: note = MISC_FEATURE - This region may encompass 3-20 residues
  VARIANT 1855..1858: note = This region may encompass one of the following sequences: GG, LS, APS, GAG, GGG, KPS, RPS, or GGGG
  VARIANT 1859..1860: note = This region may encompass one of the following sequences: S, D, T, N, L, DS, DT, LS, SS, ST, TN, or TS
  VARIANT 1863: note = A, L, P, T or V
  VARIANT 1865..1884: note = Any amino acid
  VARIANT 1865..1884: note = MISC_FEATURE - This region may encompass 3-20 residues
  VARIANT 1885..1888: note = This region may encompass one of the following sequences: GG, LS, APS, GAG, GGG, KPS, RPS, or GGGG
  VARIANT 1889..1890: note = This region may encompass one of the following sequences: S, D, T, N, L, DS, DT, LS, SS, ST, TN, or TS
  VARIANT 1893: note = A, L, P, T or V
  VARIANT 1895..1914: note = Any amino acid
  VARIANT 1895..1914: note = MISC_FEATURE - This region may encompass 3-20 residues
  VARIANT 1915..1918: note = This region may encompass one of the following sequences: GG, LS, APS, GAG, GGG, KPS, RPS, or GGGG
  VARIANT 1919..1920: note = This region may encompass one of the following sequences: S, D, T, N, L, DS, DT, LS, SS, ST, TN, or TS
  VARIANT 1923: note = A, L, P, T or V
  VARIANT 1925..1944: note = Any amino acid
  VARIANT 1925..1944: note = MISC_FEATURE - This region may encompass 3-20 residues
  VARIANT 1945..1948: note = This region may encompass one of the following sequences: GG, LS, APS, GAG, GGG, KPS, RPS, or GGGG
  VARIANT 1949..1950: note = This region may encompass one of the following sequences: S, D, T, N, L, DS, DT, LS, SS, ST, TN, or TS
  VARIANT 1953: note = A, L, P, T or V
  VARIANT 1955..1974: note = Any amino acid
  VARIANT 1955..1974: note = MISC_FEATURE - This region may encompass 3-20 residues
  VARIANT 1975..1978: note = This region may encompass one of the following sequences: GG, LS, APS, GAG, GGG, KPS, RPS, or GGGG
  VARIANT 1979..1980: note = This region may encompass one of the following sequences: S, D, T, N, L, DS, DT, LS, SS, ST, TN, or TS
  VARIANT 1983: note = A, L, P, T or V
  VARIANT 1985..2004: note = Any amino acid
  VARIANT 1985..2004: note = MISC_FEATURE - This region may encompass 3-20 residues
  VARIANT 2005..2008: note = This region may encompass one of the following sequences: GG, LS, APS, GAG, GGG, KPS, RPS, or GGGG
  VARIANT 2009..2010: note = This region may encompass one of the following sequences: S, D, T, N, L, DS, DT, LS, SS, ST, TN, or TS
  VARIANT 2013: note = A, L, P, T or V
  VARIANT 2015..2034: note = Any amino acid
  VARIANT 2015..2034: note = MISC_FEATURE - This region may encompass 3-20 residues
  VARIANT 2035..2038: note = This region may encompass one of the following sequences: GG, LS, APS, GAG, GGG, KPS, RPS, or GGGG
  VARIANT 2039..2040: note = This region may encompass one of the following sequences: S, D, T, N, L, DS, DT, LS, SS, ST, TN, or TS
  VARIANT 2043: note = A, L, P, T or V
  VARIANT 2045..2064: note = Any amino acid
  VARIANT 2045..2064: note = MISC_FEATURE - This region may encompass 3-20 residues
  VARIANT 2065..2068: note = This region may encompass one of the following sequences: GG, LS, APS, GAG, GGG, KPS, RPS, or GGGG
  VARIANT 2069..2070: note = This region may encompass one of the following sequences: S, D, T, N, L, DS, DT, LS, SS, ST, TN, or TS
  VARIANT 2073: note = A, L, P, T or V
  VARIANT 2075..2094: note = Any amino acid
  VARIANT 2075..2094: note = MISC_FEATURE - This region may encompass 3-20 residues
  VARIANT 2095..2098: note = This region may encompass one of the following sequences: GG, LS, APS, GAG, GGG, KPS, RPS, or GGGG
  VARIANT 2099..2100: note = This region may encompass one of the following sequences: S, D, T, N, L, DS, DT, LS, SS, ST, TN, or TS
  VARIANT 2103: note = A, L, P, T or V
  VARIANT 2105..2124: note = Any amino acid
  VARIANT 2105..2124: note = MISC_FEATURE - This region may encompass 3-20 residues
  VARIANT 2125..2128: note = This region may encompass one of the following sequences: GG, LS, APS, GAG, GGG, KPS, RPS, or GGGG
  VARIANT 2129..2130: note = This region may encompass one of the following sequences: S, D, T, N, L, DS, DT, LS, SS, ST, TN, or TS
  VARIANT 2133: note = A, L, P, T or V
  VARIANT 2135..2154: note = Any amino acid
  VARIANT 2135..2154: note = MISC_FEATURE - This region may encompass 3-20 residues
  VARIANT 2155..2158: note = This region may encompass one of the following sequences: GG, LS, APS, GAG, GGG, KPS, RPS, or GGGG
  VARIANT 2159..2160: note = This region may encompass one of the following sequences: S, D, T, N, L, DS, DT, LS, SS, ST, TN, or TS
  VARIANT 2163: note = A, L, P, T or V
  VARIANT 2165..2184: note = Any amino acid
  VARIANT 2165..2184: note = MISC_FEATURE - This region may encompass 3-20 residues
  VARIANT 2185..2188: note = This region may encompass one of the following sequences: GG, LS, APS, GAG, GGG, KPS, RPS, or GGGG
  VARIANT 2189..2190: note = This region may encompass one of the following sequences: S, D, T, N, L, DS, DT, LS, SS, ST, TN, or TS
  VARIANT 2193: note = A, L, P, T or V
  VARIANT 2195..2214: note = Any amino acid
  VARIANT 2195..2214: note = MISC_FEATURE - This region may encompass 3-20 residues
  VARIANT 2215..2218: note = This region may encompass one of the following sequences: GG, LS, APS, GAG, GGG, KPS, RPS, or GGGG
  VARIANT 2219..2220: note = This region may encompass one of the following sequences: S, D, T, N, L, DS, DT, LS, SS, ST, TN, or TS
  VARIANT 2223: note = A, L, P, T or V
  VARIANT 2225..2244: note = Any amino acidc
  VARIANT 2225..2244: note = MISC_FEATURE - This region may encompass 3-20 residues
  VARIANT 2245..2248: note = This region may encompass one of the following sequences: GG, LS, APS, GAG, GGG, KPS, RPS, or GGGG
  VARIANT 2249..2250: note = This region may encompass one of the following sequences: S, D, T, N, L, DS, DT, LS, SS, ST, TN, or TS
  VARIANT 2253: note = A, L, P, T or V
  VARIANT 2255..2274: note = Any amino acid
  VARIANT 2255..2274: note = MISC_FEATURE - This region may encompass 3-20 residues
  VARIANT 2275..2278: note = This region may encompass one of the following sequences: GG, LS, APS, GAG, GGG, KPS, RPS, or GGGG
  VARIANT 2279..2280: note = This region may encompass one of the following sequences: S, D, T, N, L, DS, DT, LS, SS, ST, TN, or TS
  VARIANT 2283: note = A, L, P, T or V
  VARIANT 2285..2304: note = Any amino acid
  VARIANT 2285..2304: note = MISC_FEATURE - This region may encompass 3-20 residues
  VARIANT 2305..2308: note = This region may encompass one of the following sequences: GG, LS, APS, GAG, GGG, KPS, RPS, or GGGG
  VARIANT 2309..2310: note = This region may encompass one of the following sequences: S, D, T, N, L, DS, DT, LS, SS, ST, TN, or TS
  VARIANT 2313: note = A, L, P, T or V
  VARIANT 2315..2334: note = Any amino acid
  VARIANT 2315..2334: note = MISC_FEATURE - This region may encompass 3-20 residues
  VARIANT 2335..2338: note = This region may encompass one of the following sequences: GG, LS, APS, GAG, GGG, KPS, RPS, or GGGG
  VARIANT 2339..2340: note = This region may encompass one of the following sequences: S, D, T, N, L, DS, DT, LS, SS, ST, TN, or TS
  VARIANT 2343: note = A, L, P, T or V
  VARIANT 2345..2364: note = Any amino acid
  VARIANT 2345..2364: note = MISC_FEATURE - This region may encompass 3-20 residues
  VARIANT 2365..2368: note = This region may encompass one of the following sequences: GG, LS, APS, GAG, GGG, KPS, RPS, or GGGG
  VARIANT 2369..2370: note = This region may encompass one of the following sequences: S, D, T, N, L, DS, DT, LS, SS, ST, TN, or TS
  VARIANT 2373: note = A, L, P, T or V
  VARIANT 2375..2394: note = Any amino acid
  VARIANT 2375..2394: note = MISC_FEATURE - This region may encompass 3-20 residues
  VARIANT 2395..2398: note = This region may encompass one of the following sequences: GG, LS, APS, GAG, GGG, KPS, RPS, or GGGG
  VARIANT 2399..2400: note = This region may encompass one of the following sequences: S, D, T, N, L, DS, DT, LS, SS, ST, TN, or TS
  VARIANT 2403: note = A, L, P, T or V
  VARIANT 2405..2424: note = Any amino acid
  VARIANT 2405..2424: note = MISC_FEATURE - This region may encompass 3-20 residues
  VARIANT 2425..2428: note = This region may encompass one of the following sequences: GG, LS, APS, GAG, GGG, KPS, RPS, or GGGG
  VARIANT 2429..2430: note = This region may encompass one of the following sequences: S, D, T, N, L, DS, DT, LS, SS, ST, TN, or TS
  VARIANT 2433: note = A, L, P, T or V
  VARIANT 2435..2454: note = Any amino acid
  VARIANT 2435..2454: note = MISC_FEATURE - This region may encompass 3-20 residues
  VARIANT 2455..2458: note = This region may encompass one of the following sequences: GG, LS, APS, GAG, GGG, KPS, RPS, or GGGG
  VARIANT 2459..2460: note = This region may encompass one of the following sequences: S, D, T, N, L, DS, DT, LS, SS, ST, TN, or TS
  VARIANT 2463: note = A, L, P, T or V
  VARIANT 2465..2484: note = Any amino acid
  VARIANT 2465..2484: note = MISC_FEATURE - This region may encompass 3-20 residues
  VARIANT 2485..2488: note = This region may encompass one of the following sequences: GG, LS, APS, GAG, GGG, KPS, RPS, or GGGG
  VARIANT 2489..2490: note = This region may encompass one of the following sequences: S, D, T, N, L, DS, DT, LS, SS, ST, TN, or TS
  VARIANT 2493: note = A, L, P, T or V
  VARIANT 2495..2514: note = Any amino acid
  VARIANT 2495..2514: note = MISC_FEATURE - This region may encompass 3-20 residues
  VARIANT 2515..2518: note = This region may encompass one of the following sequences: GG, LS, APS, GAG, GGG, KPS, RPS, or GGGG
  VARIANT 2519..2520: note = This region may encompass one of the following sequences: S, D, T, N, L, DS, DT, LS, SS, ST, TN, or TS
  VARIANT 2523: note = A, L, P, T or V
  VARIANT 2525..2544: note = Any amino acid
  VARIANT 2525..2544: note = MISC_FEATURE - This region may encompass 3-20 residues
  VARIANT 2545..2548: note = This region may encompass one of the following sequences: GG, LS, APS, GAG, GGG, KPS, RPS, or GGGG
  VARIANT 2549..2550: note = This region may encompass one of the following sequences: S, D, T, N, L, DS, DT, LS, SS, ST, TN, or TS
  VARIANT 2553: note = A, L, P, T or V
  VARIANT 2555..2574: note = Any amino acid
  VARIANT 2555..2574: note = MISC_FEATURE - This region may encompass 3-20 residues
  VARIANT 2575..2578: note = This region may encompass one of the following sequences: GG, LS, APS, GAG, GGG, KPS, RPS, or GGGG
  VARIANT 2579..2580: note = This region may encompass one of the following sequences: S, D, T, N, L, DS, DT, LS, SS, ST, TN, or TS
  VARIANT 2583: note = A, L, P, T or V
  VARIANT 2585..2604: note = Any amino acid
  VARIANT 2585..2604: note = MISC_FEATURE - This region may encompass 3-20 residues
  VARIANT 2605..2608: note = This region may encompass one of the following sequences: GG, LS, APS, GAG, GGG, KPS, RPS, or GGGG
  VARIANT 2609..2610: note = This region may encompass one of the following sequences: S, D, T, N, L, DS, DT, LS, SS, ST, TN, or TS
  VARIANT 2613: note = A, L, P, T or V
  VARIANT 2615..2634: note = Any amino acid
  VARIANT 2615..2634: note = MISC_FEATURE - This region may encompass 3-20 residues
  VARIANT 2635..2638: note = This region may encompass one of the following sequences: GG, LS, APS, GAG, GGG, KPS, RPS, or GGGG
  VARIANT 2639..2640: note = This region may encompass one of the following sequences: S, D, T, N, L, DS, DT, LS, SS, ST, TN, or TS
  VARIANT 2643: note = A, L, P, T or V
  VARIANT 2645..2664: note = Any amino acid
  VARIANT 2645..2664: note = MISC_FEATURE - This region may encompass 3-20 residues
  VARIANT 2665..2668: note = This region may encompass one of the following sequences: GG, LS, APS, GAG, GGG, KPS, RPS, or GGGG
  VARIANT 2669..2670: note = This region may encompass one of the following sequences: S, D, T, N, L, DS, DT, LS, SS, ST, TN, or TS
  VARIANT 2673: note = A, L, P, T or V
  VARIANT 2675..2694: note = Any amino acid
  VARIANT 2675..2694: note = MISC_FEATURE - This region may encompass 3-20 residues
  VARIANT 2695..2698: note = This region may encompass one of the following sequences: GG, LS, APS, GAG, GGG, KPS, RPS, or GGGG
  VARIANT 2699..2700: note = This region may encompass one of the following sequences: S, D, T, N, L, DS, DT, LS, SS, ST, TN, or TS
  VARIANT 2703: note = A, L, P, T or V
  VARIANT 2705..2724: note = Any amino acid
  VARIANT 2705..2724: note = MISC_FEATURE - This region may encompass 3-20 residues
  VARIANT 2725..2728: note = This region may encompass one of the following sequences: GG, LS, APS, GAG, GGG, KPS, RPS, or GGGG
  VARIANT 2729..2730: note = This region may encompass one of the following sequences: S, D, T, N, L, DS, DT, LS, SS, ST, TN, or TS
  VARIANT 2733: note = A, L, P, T or V
  VARIANT 2735..2754: note = Any amino acid
  VARIANT 2735..2754: note = MISC_FEATURE - This region may encompass 3-20 residues
  VARIANT 2755..2758: note = This region may encompass one of the following sequences: GG, LS, APS, GAG, GGG, KPS, RPS, or GGGG
  VARIANT 2759..2760: note = This region may encompass one of the following sequences: S, D, T, N, L, DS, DT, LS, SS, ST, TN, or TS
  VARIANT 2763: note = A, L, P, T or V
  VARIANT 2765..2784: note = Any amino acid
  VARIANT 2765..2784: note = MISC_FEATURE - This region may encompass 3-20 residues
  VARIANT 2785..2788: note = This region may encompass one of the following sequences: GG, LS, APS, GAG, GGG, KPS, RPS, or GGGG
  VARIANT 2789..2790: note = This region may encompass one of the following sequences: S, D, T, N, L, DS, DT, LS, SS, ST, TN, or TS
  VARIANT 2793: note = A, L, P, T or V
  VARIANT 2795..2814: note = Any amino acid
  VARIANT 2795..2814: note = MISC_FEATURE - This region may encompass 3-20 residues
  VARIANT 2815..2818: note = This region may encompass one of the following sequences: GG, LS, APS, GAG, GGG, KPS, RPS, or GGGG
  VARIANT 2819..2820: note = This region may encompass one of the following sequences: S, D, T, N, L, DS, DT, LS, SS, ST, TN, or TS
  VARIANT 2823: note = A, L, P, T or V
  VARIANT 2825..2844: note = Any amino acid
  VARIANT 2825..2844: note = MISC_FEATURE - This region may encompass 3-20 residues
  VARIANT 2845..2848: note = This region may encompass one of the following sequences: GG, LS, APS, GAG, GGG, KPS, RPS, or GGGG
  VARIANT 2849..2850: note = This region may encompass one of the following sequences: S, D, T, N, L, DS, DT, LS, SS, ST, TN, or TS
  VARIANT 2853: note = A, L, P, T or V
  VARIANT 2855..2874: note = Any amino acid
  VARIANT 2855..2874: note = MISC_FEATURE - This region may encompass 3-20 residues
  VARIANT 2875..2878: note = This region may encompass one of the following sequences: GG, LS, APS, GAG, GGG, KPS, RPS, or GGGG
  VARIANT 2879..2880: note = This region may encompass one of the following sequences: S, D, T, N, L, DS, DT, LS, SS, ST, TN, or TS
  VARIANT 2883: note = A, L, P, T or V
  VARIANT 2885..2904: note = Any amino acid
  VARIANT 2885..2904: note = MISC_FEATURE - This region may encompass 3-20 residues
  VARIANT 2905..2908: note = This region may encompass one of the following sequences: GG, LS, APS, GAG, GGG, KPS, RPS, or GGGG
  VARIANT 2909..2910: note = This region may encompass one of the following sequences: S, D, T, N, L, DS, DT, LS, SS, ST, TN, or TS
  VARIANT 2913: note = A, L, P, T or V
  VARIANT 2915..2934: note = Any amino acid
  VARIANT 2915..2934: note = MISC_FEATURE - This region may encompass 3-20 residues
  VARIANT 2935..2938: note = This region may encompass one of the following sequences: GG, LS, APS, GAG, GGG, KPS, RPS, or GGGG
  VARIANT 2939..2940: note = This region may encompass one of the following sequences: S, D, T, N, L, DS, DT, LS, SS, ST, TN, or TS
  VARIANT 2943: note = A, L, P, T or V
  VARIANT 2945..2964: note = Any amino acid
  VARIANT 2945..2964: note = MISC_FEATURE - This region may encompass 3-20 residues
  VARIANT 2965..2968: note = This region may encompass one of the following sequences: GG, LS, APS, GAG, GGG, KPS, RPS, or GGGG
  VARIANT 2969..2970: note = This region may encompass one of the following sequences: S, D, T, N, L, DS, DT, LS, SS, ST, TN, or TS
  VARIANT 2973: note = A, L, P, T or V
  VARIANT 2975..2994: note = Any amino acid
  VARIANT 2975..2994: note = MISC_FEATURE - This region may encompass 3-20 residues
  VARIANT 2995..2998: note = This region may encompass one of the following sequences: GG, LS, APS, GAG, GGG, KPS, RPS, or GGGG
  VARIANT 2999..3000: note = This region may encompass one of the following sequences: S, D, T, N, L, DS, DT, LS, SS, ST, TN, or TS
  VARIANT 1..3000: note = MISC_FEATURE - This region may encompass 3-20 residues
YGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXXYGXPXXXXXXXXXXXXXXXXXXXXXXXXXX

SEQ ID NO: 65 - protein, 4000 aa, synthetic construct
  REGION 1..4000: note = Description of Artificial Sequence: Synthetic polypeptide
  VARIANT 1..4000: note = MISC_FEATURE - This sequence may encompass 1-100 "Tyr Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa Xaa" repeating units
  VARIANT 2..5: note = Any amino acid
  VARIANT 2..5: note = MISC_FEATURE - This region may encompass 1-4 residues or this region may be absent in its entirety
  VARIANT 6..10: note = This region may encompass one of the following sequences: GQ, GN, RPG, RPGGQ, RPGGN, SSS, SKG, or SN
  VARIANT 11..40: note = Any amino acid
  VARIANT 11..40: note = MISC_FEATURE - This region may encompass 4-30 residues
  VARIANT 42..45: note = Any amino acid
  VARIANT 42..45: note = MISC_FEATURE - This region may encompass 1-4 residues or this region may be absent in its entirety
  VARIANT 46..50: note = This region may encompass one of the following sequences: GQ, GN, RPG, RPGGQ, RPGGN, SSS, SKG, or SN
  VARIANT 51..80: note = Any amino acid
  VARIANT 51..80: note = MISC_FEATURE - This region may encompass 4-30 residues
  VARIANT 82..85: note = Any amino acid
  VARIANT 82..85: note = MISC_FEATURE - This region may encompass 1-4 residues or this region may be absent in its entirety
  VARIANT 86..90: note = This region may encompass one of the following sequences: GQ, GN, RPG, RPGGQ, RPGGN, SSS, SKG, or SN
  VARIANT 91..120: note = Any amino acid
  VARIANT 91..120: note = MISC_FEATURE - This region may encompass 4-30 residues
  VARIANT 122..125: note = Any amino acid
  VARIANT 122..125: note = MISC_FEATURE - This region may encompass 1-4 residues or this region may be absent in its entirety
  VARIANT 126..130: note = This region may encompass one of the following sequences: GQ, GN, RPG, RPGGQ, RPGGN, SSS, SKG, or SN
  VARIANT 131..160: note = Any amino acid
  VARIANT 131..160: note = MISC_FEATURE - This region may encompass 4-30 residues
  VARIANT 162..165: note = Any amino acid
  VARIANT 162..165: note = MISC_FEATURE - This region may encompass 1-4 residues or this region may be absent in its entirety
  VARIANT 166..170: note = This region may encompass one of the following sequences: GQ, GN, RPG, RPGGQ, RPGGN, SSS, SKG, or SN
  VARIANT 171..200: note = Any amino acid
  VARIANT 171..200: note = MISC_FEATURE - This region may encompass 4-30 residues
  VARIANT 202..205: note = Any amino acid
  VARIANT 202..205: note = MISC_FEATURE - This region may encompass 1-4 residues or this region may be absent in its entirety
  VARIANT 206..210: note = This region may encompass one of the following sequences: GQ, GN, RPG, RPGGQ, RPGGN, SSS, SKG, or SN
  VARIANT 211..240: note = Any amino acid
  VARIANT 211..240: note = MISC_FEATURE - This region may encompass 4-30 residues
  VARIANT 242..245: note = Any amino acid
  VARIANT 242..245: note = MISC_FEATURE - This region may encompass 1-4 residues or this region may be absent in its entirety
  VARIANT 246..250: note = This region may encompass one of the following sequences: GQ, GN, RPG, RPGGQ, RPGGN, SSS, SKG, or SN
  VARIANT 251..280: note = Any amino acid
  VARIANT 251..280: note = MISC_FEATURE - This region may encompass 4-30 residues
  VARIANT 282..285: note = Any amino acid
  VARIANT 282..285: note = MISC_FEATURE - This region may encompass 1-4 residues or this region may be absent in its entirety
  VARIANT 286..290: note = This region may encompass one of the following sequences: GQ, GN, RPG, RPGGQ, RPGGN, SSS, SKG, or SN
  VARIANT 291..320: note = Any amino acid
  VARIANT 291..320: note = MISC_FEATURE - This region may encompass 4-30 residues
  VARIANT 322..325: note = Any amino acid
  VARIANT 322..325: note = MISC_FEATURE - This region may encompass 1-4 residues or this region may be absent in its entirety
  VARIANT 326..330: note = This region may encompass one of the following sequences: GQ, GN, RPG, RPGGQ, RPGGN, SSS, SKG, or SN
  VARIANT 331..360: note = Any amino acid
  VARIANT 331..360: note = MISC_FEATURE - This region may encompass 4-30 residues
  VARIANT 362..365: note = Any amino acid
  VARIANT 362..365: note = MISC_FEATURE - This region may encompass 1-4 residues or this region may be absent in its entirety
  VARIANT 366..370: note = This region may encompass one of the following sequences: GQ, GN, RPG, RPGGQ, RPGGN, SSS, SKG, or SN
  VARIANT 371..400: note = Any amino acid
  VARIANT 371..400: note = MISC_FEATURE - This region may encompass 4-30 residues
  VARIANT 402..405: note = Any amino acid
  VARIANT 402..405: note = MISC_FEATURE - This region may encompass 1-4 residues or this region may be absent in its entirety
  VARIANT 406..410: note = This region may encompass one of the following sequences: GQ, GN, RPG, RPGGQ, RPGGN, SSS, SKG, or SN
  VARIANT 411..440: note = Any amino acid
  VARIANT 411..440: note = MISC_FEATURE - This region may encompass 4-30 residues
  VARIANT 442..445: note = Any amino acid
  VARIANT 442..445: note = MISC_FEATURE - This region may encompass 1-4 residues or this region may be absent in its entirety
  VARIANT 446..450: note = This region may encompass one of the following sequences: GQ, GN, RPG, RPGGQ, RPGGN, SSS, SKG, or SN
  VARIANT 451..480: note = Any amino acid
  VARIANT 451..480: note = MISC_FEATURE - This region may encompass 4-30 residues
  VARIANT 482..485: note = Any amino acid
  VARIANT 482..485: note = MISC_FEATURE - This region may encompass 1-4 residues or this region may be absent in its entirety
  VARIANT 486..490: note = This region may encompass one of the following sequences: GQ, GN, RPG, RPGGQ, RPGGN, SSS, SKG, or SN
  VARIANT 491..520: note = Any amino acid
  VARIANT 491..520: note = MISC_FEATURE - This region may encompass 4-30 residues
  VARIANT 522..525: note = Any amino acid
  VARIANT 522..525: note = MISC_FEATURE - This region may encompass 1-4 residues or this region may be absent in its entirety
  VARIANT 526..530: note = This region may encompass one of the following sequences: GQ, GN, RPG, RPGGQ, RPGGN, SSS, SKG, or SN
  VARIANT 531..560: note = Any amino acid
  VARIANT 531..560: note = MISC_FEATURE - This region may encompass 4-30 residues
  VARIANT 562..565: note = Any amino acid
  VARIANT 562..565: note = MISC_FEATURE - This region may encompass 1-4 residues or this region may be absent in its entirety
  VARIANT 566..570: note = This region may encompass one of the following sequences: GQ, GN, RPG, RPGGQ, RPGGN, SSS, SKG, or SN
  VARIANT 571..600: note = Any amino acid
  VARIANT 571..600: note = MISC_FEATURE - This region may encompass 4-30 residues
  VARIANT 602..605: note = Any amino acid
  VARIANT 602..605: note = MISC_FEATURE - This region may encompass 1-4 residues or this region may be absent in its entirety
  VARIANT 606..610: note = This region may encompass one of the following sequences: GQ, GN, RPG, RPGGQ, RPGGN, SSS, SKG, or SN
  VARIANT 611..640: note = Any amino acid
  VARIANT 611..640: note = MISC_FEATURE - This region may encompass 4-30 residues
  VARIANT 642..645: note = Any amino acid
  VARIANT 642..645: note = MISC_FEATURE - This region may encompass 1-4 residues or this region may be absent in its entirety
  VARIANT 646..650: note = This region may encompass one of the following sequences: GQ, GN, RPG, RPGGQ, RPGGN, SSS, SKG, or SN
  VARIANT 651..680: note = Any amino acid
  VARIANT 651..680: note = MISC_FEATURE - This region may encompass 4-30 residues
  VARIANT 682..685: note = Any amino acid
  VARIANT 682..685: note = MISC_FEATURE - This region may encompass 1-4 residues or this region may be absent in its entirety
  VARIANT 686..690: note = This region may encompass one of the following sequences: GQ, GN, RPG, RPGGQ, RPGGN, SSS, SKG, or SN
  VARIANT 691..720: note = Any amino acid
  VARIANT 691..720: note = MISC_FEATURE - This region may encompass 4-30 residues
  VARIANT 722..725: note = Any amino acid
  VARIANT 722..725: note = MISC_FEATURE - This region may encompass 1-4 residues or this region may be absent in its entirety
  VARIANT 726..730: note = This region may encompass one of the following sequences: GQ, GN, RPG, RPGGQ, RPGGN, SSS, SKG, or SN
  VARIANT 731..760: note = Any amino acid
  VARIANT 731..760: note = MISC_FEATURE - This region may encompass 4-30 residues
  VARIANT 762..765: note = Any amino acid
  VARIANT 762..765: note = MISC_FEATURE - This region may encompass 1-4 residues or this region may be absent in its entirety
  VARIANT 766..770: note = This region may encompass one of the following sequences: GQ, GN, RPG, RPGGQ, RPGGN, SSS, SKG, or SN
  VARIANT 771..800: note = Any amino acid
  VARIANT 771..800: note = MISC_FEATURE - This region may encompass 4-30 residues
  VARIANT 802..805: note = Any amino acid
  VARIANT 802..805: note = MISC_FEATURE - This region may encompass 1-4 residues or this region may be absent in its entirety
  VARIANT 806..810: note = This region may encompass one of the following sequences: GQ, GN, RPG, RPGGQ, RPGGN, SSS, SKG, or SN
  VARIANT 811..840: note = Any amino acid
  VARIANT 811..840: note = MISC_FEATURE - This region may encompass 4-30 residues
  VARIANT 842..845: note = Any amino acid
  VARIANT 842..845: note = MISC_FEATURE - This region may encompass 1-4 residues or this region may be absent in its entirety
  VARIANT 846..850: note = This region may encompass one of the following sequences: GQ, GN, RPG, RPGGQ, RPGGN, SSS, SKG, or SN
  VARIANT 851..880: note = Any amino acid
  VARIANT 851..880: note = MISC_FEATURE - This region may encompass 4-30 residues
  VARIANT 882..885: note = Any amino acid
  VARIANT 882..885: note = MISC_FEATURE - This region may encompass 1-4 residues or this region may be absent in its entirety
  VARIANT 886..890: note = This region may encompass one of the following sequences: GQ, GN, RPG, RPGGQ, RPGGN, SSS, SKG, or SN
  VARIANT 891..920: note = Any amino acid
  VARIANT 891..920: note = MISC_FEATURE - This region may encompass 4-30 residues
  VARIANT 922..925: note = Any amino acid
  VARIANT 922..925: note = MISC_FEATURE - This region may encompass 1-4 residues or this region may be absent in its entirety
  VARIANT 926..930: note = This region may encompass one of the following sequences: GQ, GN, RPG, RPGGQ, RPGGN, SSS, SKG, or SN
  VARIANT 931..960: note = Any amino acid
  VARIANT 931..960: note = MISC_FEATURE - This region may encompass 4-30 residues
  VARIANT 962..965: note = Any amino acid
  VARIANT 962..965: note = MISC_FEATURE - This region may encompass 1-4 residues or this region may be absent in its entirety
  VARIANT 966..970: note = This region may encompass one of the following sequences: GQ, GN, RPG, RPGGQ, RPGGN, SSS, SKG, or SN
  VARIANT 971..1000: note = Any amino acid
  VARIANT 971..1000: note = MISC_FEATURE - This region may encompass 4-30 residues
  VARIANT 1002..1005: note = Any amino acid
  VARIANT 1002..1005: note = MISC_FEATURE - This region may encompass 1-4 residues or this region may be absent in its entirety
  VARIANT 1006..1010: note = This region may encompass one of the following sequences: GQ, GN, RPG, RPGGQ, RPGGN, SSS, SKG, or SN
  VARIANT 1011..1040: note = Any amino acid
  VARIANT 1011..1040: note = MISC_FEATURE - This region may encompass 4-30 residues
  VARIANT 1042..1045: note = Any amino acid
  VARIANT 1042..1045: note = MISC_FEATURE - This region may encompass 1-4 residues or this region may be absent in its entirety
  VARIANT 1046..1050: note = This region may encompass one of the following sequences: GQ, GN, RPG, RPGGQ, RPGGN, SSS, SKG, or SN
  VARIANT 1051..1080: note = Any amino acid
  VARIANT 1051..1080: note = MISC_FEATURE - This region may encompass 4-30 residues
  VARIANT 1082..1085: note = Any amino acid
  VARIANT 1082..1085: note = MISC_FEATURE - This region may encompass 1-4 residues or this region may be absent in its entirety
  VARIANT 1086..1090: note = This region may encompass one of the following sequences: GQ, GN, RPG, RPGGQ, RPGGN, SSS, SKG, or SN
  VARIANT 1091..1120: note = Any amino acid
  VARIANT 1091..1120: note = MISC_FEATURE - This region may encompass 4-30 residues
  VARIANT 1122..1125: note = Any amino acid
  VARIANT 1122..1125: note = MISC_FEATURE - This region may encompass 1-4 residues or this region may be absent in its entirety
  VARIANT 1126..1130: note = This region may encompass one of the following sequences: GQ, GN, RPG, RPGGQ, RPGGN, SSS, SKG, or SN
  VARIANT 1131..1160: note = Any amino acid
  VARIANT 1131..1160: note = MISC_FEATURE - This region may encompass 4-30 residues
  VARIANT 1162..1165: note = Any amino acid
  VARIANT 1162..1165: note = MISC_FEATURE - This region may encompass 1-4 residues or this region may be absent in its entirety
  VARIANT 1166..1170: note = This region may encompass one of the following sequences: GQ, GN, RPG, RPGGQ, RPGGN, SSS, SKG, or SN
  VARIANT 1171..1200: note = Any amino acid
  VARIANT 1171..1200: note = MISC_FEATURE - This region may encompass 4-30 residues
  VARIANT 1202..1205: note = Any amino acid
  VARIANT 1202..1205: note = MISC_FEATURE - This region may encompass 1-4 residues or this region may be absent in its entirety
  VARIANT 1206..1210: note = This region may encompass one of the following sequences: GQ, GN, RPG, RPGGQ, RPGGN, SSS, SKG, or SN
  VARIANT 1211..1240: note = Any amino acid
  VARIANT 1211..1240: note = MISC_FEATURE - This region may encompass 4-30 residues
  VARIANT 1242..1245: note = Any amino acid
  VARIANT 1242..1245: note = MISC_FEATURE - This region may encompass 1-4 residues or this region may be absent in its entirety
  VARIANT 1246..1250: note = This region may encompass one of the following sequences: GQ, GN, RPG, RPGGQ, RPGGN, SSS, SKG, or SN
  VARIANT 1251..1280: note = Any amino acid
  VARIANT 1251..1280: note = MISC_FEATURE - This region may encompass 4-30 residues
  VARIANT 1282..1285: note = Any amino acid
  VARIANT 1282..1285: note = MISC_FEATURE - This region may encompass 1-4 residues or this region may be absent in its entirety
  VARIANT 1286..1290: note = This region may encompass one of the following sequences: GQ, GN, RPG, RPGGQ, RPGGN, SSS, SKG, or SN
  VARIANT 1291..1320: note = Any amino acid
  VARIANT 1291..1320: note = MISC_FEATURE - This region may encompass 4-30 residues
  VARIANT 1322..1325: note = Any amino acid
  VARIANT 1322..1325: note = MISC_FEATURE - This region may encompass 1-4 residues or this region may be absent in its entirety
  VARIANT 1326..1330: note = This region may encompass one of the following sequences: GQ, GN, RPG, RPGGQ, RPGGN, SSS, SKG, or SN
  VARIANT 1331..1360: note = Any amino acid
  VARIANT 1331..1360: note = MISC_FEATURE - This region may encompass 4-30 residues
  VARIANT 1362..1365: note = Any amino acid
  VARIANT 1362..1365: note = MISC_FEATURE - This region may encompass 1-4 residues or this region may be absent in its entirety
  VARIANT 1366..1370: note = This region may encompass one of the following sequences: GQ, GN, RPG, RPGGQ, RPGGN, SSS, SKG, or SN
  VARIANT 1371..1400: note = Any amino acid
  VARIANT 1371..1400: note = MISC_FEATURE - This region may encompass 4-30 residues
  VARIANT 1402..1405: note = Any amino acid
  VARIANT 1402..1405: note = MISC_FEATURE - This region may encompass 1-4 residues or this region may be absent in its entirety
  VARIANT 1406..1410: note = This region may encompass one of the following sequences: GQ, GN, RPG, RPGGQ, RPGGN, SSS, SKG, or SN
  VARIANT 1411..1440: note = Any amino acid
  VARIANT 1411..1440: note = MISC_FEATURE - This region may encompass 4-30 residues
  VARIANT 1442..1445: note = Any amino acid
  VARIANT 1442..1445: note = MISC_FEATURE - This region may encompass 1-4 residues or this region may be absent in its entirety
  VARIANT 1446..1450: note = This region may encompass one of the following sequences: GQ, GN, RPG, RPGGQ, RPGGN, SSS, SKG, or SN
  VARIANT 1451..1480: note = Any amino acid
  VARIANT 1451..1480: note = MISC_FEATURE - This region may encompass 4-30 residues
  VARIANT 1482..1485: note = Any amino acid
  VARIANT 1482..1485: note = MISC_FEATURE - This region may encompass 1-4 residues or this region may be absent in its entirety
  VARIANT 1486..1490: note = This region may encompass one of the following sequences: GQ, GN, RPG, RPGGQ, RPGGN, SSS, SKG, or SN
  VARIANT 1491..1520: note = Any amino acid
  VARIANT 1491..1520: note = MISC_FEATURE - This region may encompass 4-30 residues
  VARIANT 1522..1525: note = Any amino acid
  VARIANT 1522..1525: note = MISC_FEATURE - This region may encompass 1-4 residues or this region may be absent in its entirety
  VARIANT 1526..1530: note = This region may encompass one of the following sequences: GQ, GN, RPG, RPGGQ, RPGGN, SSS, SKG, or SN
  VARIANT 1531..1560: note = Any amino acid
  VARIANT 1531..1560: note = MISC_FEATURE - This region may encompass 4-30 residues
  VARIANT 1562..1565: note = Any amino acid
  VARIANT 1562..1565: note = MISC_FEATURE - This region may encompass 1-4 residues or this region may be absent in its entirety
  VARIANT 1566..1570: note = This region may encompass one of the following sequences: GQ, GN, RPG, RPGGQ, RPGGN, SSS, SKG, or SN
  VARIANT 1571..1600: note = Any amino acid
  VARIANT 1571..1600: note = MISC_FEATURE - This region may encompass 4-30 residues
  VARIANT 1602..1605: note = Any amino acid
  VARIANT 1602..1605: note = MISC_FEATURE - This region may encompass 1-4 residues or this region may be absent in its entirety
  VARIANT 1606..1610: note = This region may encompass one of the following sequences: GQ, GN, RPG, RPGGQ, RPGGN, SSS, SKG, or SN
  VARIANT 1611..1640: note = Any amino acid
  VARIANT 1611..1640: note = MISC_FEATURE - This region may encompass 4-30 residues
  VARIANT 1642..1645: note = Any amino acid
  VARIANT 1642..1645: note = MISC_FEATURE - This region may encompass 1-4 residues or this region may be absent in its entirety
  VARIANT 1646..1650: note = This region may encompass one of the following sequences: GQ, GN, RPG, RPGGQ, RPGGN, SSS, SKG, or SN
  VARIANT 1651..1680: note = Any amino acid
  VARIANT 1651..1680: note = MISC_FEATURE - This region may encompass 4-30 residues
  VARIANT 1682..1685: note = Any amino acid
  VARIANT 1682..1685: note = MISC_FEATURE - This region may encompass 1-4 residues or this region may be absent in its entirety
  VARIANT 1686..1690: note = This region may encompass one of the following sequences: GQ, GN, RPG, RPGGQ, RPGGN, SSS, SKG, or SN
  VARIANT 1691..1720: note = Any amino acid
  VARIANT 1691..1720: note = MISC_FEATURE - This region may encompass 4-30 residues
  VARIANT 1722..1725: note = Any amino acid
  VARIANT 1722..1725: note = MISC_FEATURE - This region may encompass 1-4 residues or this region may be absent in its entirety
  VARIANT 1726..1730: note = This region may encompass one of the following sequences: GQ, GN, RPG, RPGGQ, RPGGN, SSS, SKG, or SN
  VARIANT 1731..1760: note = Any amino acid
  VARIANT 1731..1760: note = MISC_FEATURE - This region may encompass 4-30 residues
  VARIANT 1762..1765: note = Any amino acid
  VARIANT 1762..1765: note = MISC_FEATURE - This region may encompass 1-4 residues or this region may be absent in its entirety
  VARIANT 1766..1770: note = This region may encompass one of the following sequences: GQ, GN, RPG, RPGGQ, RPGGN, SSS, SKG, or SN
  VARIANT 1771..1800: note = Any amino acid
  VARIANT 1771..1800: note = MISC_FEATURE - This region may encompass 4-30 residues
  VARIANT 1802..1805: note = Any amino acid
  VARIANT 1802..1805: note = MISC_FEATURE - This region may encompass 1-4 residues or this region may be absent in its entirety
  VARIANT 1806..1810: note = This region may encompass one of the following sequences: GQ, GN, RPG, RPGGQ, RPGGN, SSS, SKG, or SN
  VARIANT 1811..1840: note = Any amino acid
  VARIANT 1811..1840: note = MISC_FEATURE - This region may encompass 4-30 residues
  VARIANT 1842..1845: note = Any amino acid
  VARIANT 1842..1845: note = MISC_FEATURE - This region may encompass 1-4 residues or this region may be absent in its entirety
  VARIANT 1846..1850: note = This region may encompass one of the following sequences: GQ, GN, RPG, RPGGQ, RPGGN, SSS, SKG, or SN
  VARIANT 1851..1880: note = Any amino acid
  VARIANT 1851..1880: note = MISC_FEATURE - This region may encompass 4-30 residues
  VARIANT 1882..1885: note = Any amino acid
  VARIANT 1882..1885: note = MISC_FEATURE - This region may encompass 1-4 residues or this region may be absent in its entirety
  VARIANT 1886..1890: note = This region may encompass one of the following sequences: GQ, GN, RPG, RPGGQ, RPGGN, SSS, SKG, or SN
  VARIANT 1891..1920: note = Any amino acid
  VARIANT 1891..1920: note = MISC_FEATURE - This region may encompass 4-30 residues
  VARIANT 1922..1925: note = Any amino acid
  VARIANT 1922..1925: note = MISC_FEATURE - This region may encompass 1-4 residues or this region may be absent in its entirety
  VARIANT 1926..1930: note = This region may encompass one of the following sequences: GQ, GN, RPG, RPGGQ, RPGGN, SSS, SKG, or SN
  VARIANT 1931..1960: note = Any amino acid
  VARIANT 1931..1960: note = MISC_FEATURE - This region may encompass 4-30 residues
  VARIANT 1962..1965: note = Any amino acid
  VARIANT 1962..1965: note = MISC_FEATURE - This region may encompass 1-4 residues or this region may be absent in its entirety
  VARIANT 1966..1970: note = This region may encompass one of the following sequences: GQ, GN, RPG, RPGGQ, RPGGN, SSS, SKG, or SN
  VARIANT 1971..2000: note = Any amino acid
  VARIANT 1971..2000: note = MISC_FEATURE - This region may encompass 4-30 residues
  VARIANT 2002..2005: note = Any amino acid
  VARIANT 2002..2005: note = MISC_FEATURE - This region may encompass 1-4 residues or this region may be absent in its entirety
  VARIANT 2006..2010: note = This region may encompass one of the following sequences: GQ, GN, RPG, RPGGQ, RPGGN, SSS, SKG, or SN
  VARIANT 2011..2040: note = Any amino acid
  VARIANT 2011..2040: note = MISC_FEATURE - This region may encompass 4-30 residues
  VARIANT 2042..2045: note = Any amino acid
  VARIANT 2042..2045: note = MISC_FEATURE - This region may encompass 1-4 residues or this region may be absent in its entirety
  VARIANT 2046..2050: note = This region may encompass one of the following sequences: GQ, GN, RPG, RPGGQ, RPGGN, SSS, SKG, or SN
  VARIANT 2051..2080: note = Any amino acid
  VARIANT 2051..2080: note = MISC_FEATURE - This region may encompass 4-30 residues
  VARIANT 2082..2085: note = Any amino acid
  VARIANT 2082..2085: note = MISC_FEATURE - This region may encompass 1-4 residues or this region may be absent in its entirety
  VARIANT 2086..2090: note = This region may encompass one of the following sequences: GQ, GN, RPG, RPGGQ, RPGGN, SSS, SKG, or SN
  VARIANT 2091..2120: note = Any amino acid
  VARIANT 2091..2120: note = MISC_FEATURE - This region may encompass 4-30 residues
  VARIANT 2122..2125: note = Any amino acid
  VARIANT 2122..2125: note = MISC_FEATURE - This region may encompass 1-4 residues or this region may be absent in its entirety
  VARIANT 2126..2130: note = This region may encompass one of the following sequences: GQ, GN, RPG, RPGGQ, RPGGN, SSS, SKG, or SN
  VARIANT 2131..2160: note = Any amino acid
  VARIANT 2131..2160: note = MISC_FEATURE - This region may encompass 4-30 residues
  VARIANT 2162..2165: note = Any amino acid
  VARIANT 2162..2165: note = MISC_FEATURE - This region may encompass 1-4 residues or this region may be absent in its entirety
  VARIANT 2166..2170: note = This region may encompass one of the following sequences: GQ, GN, RPG, RPGGQ, RPGGN, SSS, SKG, or SN
  VARIANT 2171..2200: note = Any amino acid
  VARIANT 2171..2200: note = MISC_FEATURE - This region may encompass 4-30 residues
  VARIANT 2202..2205: note = Any amino acid
  VARIANT 2202..2205: note = MISC_FEATURE - This region may encompass 1-4 residues or this region may be absent in its entirety
  VARIANT 2206..2210: note = This region may encompass one of the following sequences: GQ, GN, RPG, RPGGQ, RPGGN, SSS, SKG, or SN
  VARIANT 2211..2240: note = Any amino acid
  VARIANT 2211..2240: note = MISC_FEATURE - This region may encompass 4-30 residues
  VARIANT 2242..2245: note = Any amino acid
  VARIANT 2242..2245: note = MISC_FEATURE - This region may encompass 1-4 residues or this region may be absent in its entirety
  VARIANT 2246..2250: note = This region may encompass one of the following sequences: GQ, GN, RPG, RPGGQ, RPGGN, SSS, SKG, or SN
  VARIANT 2251..2280: note = Any amino acid
  VARIANT 2251..2280: note = MISC_FEATURE - This region may encompass 4-30 residues
  VARIANT 2282..2285: note = Any amino acid
  VARIANT 2282..2285: note = MISC_FEATURE - This region may encompass 1-4 residues or this region may be absent in its entirety
  VARIANT 2286..2290: note = This region may encompass one of the following sequences: GQ, GN, RPG, RPGGQ, RPGGN, SSS, SKG, or SN
  VARIANT 2291..2320: note = Any amino acid
  VARIANT 2291..2320: note = MISC_FEATURE - This region may encompass 4-30 residues
  VARIANT 2322..2325: note = Any amino acid
  VARIANT 2322..2325: note = MISC_FEATURE - This region may encompass 1-4 residues or this region may be absent in its entirety
  VARIANT 2326..2330: note = This region may encompass one of the following sequences: GQ, GN, RPG, RPGGQ, RPGGN, SSS, SKG, or SN
  VARIANT 2331..2360: note = Any amino acid
  VARIANT 2331..2360: note = MISC_FEATURE - This region may encompass 4-30 residues
  VARIANT 2362..2365: note = Any amino acid
  VARIANT 2362..2365: note = MISC_FEATURE - This region may encompass 1-4 residues or this region may be absent in its entirety
  VARIANT 2366..2370: note = This region may encompass one of the following sequences: GQ, GN, RPG, RPGGQ, RPGGN, SSS, SKG, or SN
  VARIANT 2371..2400: note = Any amino acid
  VARIANT 2371..2400: note = MISC_FEATURE - This region may encompass 4-30 residues
  VARIANT 2402..2405: note = Any amino acid
  VARIANT 2402..2405: note = MISC_FEATURE - This region may encompass 1-4 residues or this region may be absent in its entirety
  VARIANT 2406..2410: note = This region may encompass one of the following sequences: GQ, GN, RPG, RPGGQ, RPGGN, SSS, SKG, or SN
  VARIANT 2411..2440: note = Any amino acid
  VARIANT 2411..2440: note = MISC_FEATURE - This region may encompass 4-30 residues
  VARIANT 2442..2445: note = Any amino acid
  VARIANT 2442..2445: note = MISC_FEATURE - This region may encompass 1-4 residues or this region may be absent in its entirety
  VARIANT 2446..2450: note = This region may encompass one of the following sequences: GQ, GN, RPG, RPGGQ, RPGGN, SSS, SKG, or SN
  VARIANT 2451..2480: note = Any amino acid
  VARIANT 2451..2480: note = MISC_FEATURE - This region may encompass 4-30 residues
  VARIANT 2482..2485: note = Any amino acid
  VARIANT 2482..2485: note = MISC_FEATURE - This region may encompass 1-4 residues or this region may be absent in its entirety
  VARIANT 2486..2490: note = This region may encompass one of the following sequences: GQ, GN, RPG, RPGGQ, RPGGN, SSS, SKG, or SN
  VARIANT 2491..2520: note = Any amino acid
  VARIANT 2491..2520: note = MISC_FEATURE - This region may encompass 4-30 residues
  VARIANT 2522..2525: note = Any amino acid
  VARIANT 2522..2525: note = MISC_FEATURE - This region may encompass 1-4 residues or this region may be absent in its entirety
  VARIANT 2526..2530: note = This region may encompass one of the following sequences: GQ, GN, RPG, RPGGQ, RPGGN, SSS, SKG, or SN
  VARIANT 2531..2560: note = Any amino acid
  VARIANT 2531..2560: note = MISC_FEATURE - This region may encompass 4-30 residues
  VARIANT 2562..2565: note = Any amino acid
  VARIANT 2562..2565: note = MISC_FEATURE - This region may encompass 1-4 residues or this region may be absent in its entirety
  VARIANT 2566..2570: note = This region may encompass one of the following sequences: GQ, GN, RPG, RPGGQ, RPGGN, SSS, SKG, or SN
  VARIANT 2571..2600: note = Any amino acid
  VARIANT 2571..2600: note = MISC_FEATURE - This region may encompass 4-30 residues
  VARIANT 2602..2605: note = Any amino acid
  VARIANT 2602..2605: note = MISC_FEATURE - This region may encompass 1-4 residues or this region may be absent in its entirety
  VARIANT 2606..2610: note = This region may encompass one of the following sequences: GQ, GN, RPG, RPGGQ, RPGGN, SSS, SKG, or SN
  VARIANT 2611..2640: note = Any amino acid
  VARIANT 2611..2640: note = MISC_FEATURE - This region may encompass 4-30 residues
  VARIANT 2642..2645: note = Any amino acid
  VARIANT 2642..2645: note = MISC_FEATURE - This region may encompass 1-4 residues or this region may be absent in its entirety
  VARIANT 2646..2650: note = This region may encompass one of the following sequences: GQ, GN, RPG, RPGGQ, RPGGN, SSS, SKG, or SN
  VARIANT 2651..2680: note = Any amino acid
  VARIANT 2651..2680: note = MISC_FEATURE - This region may encompass 4-30 residues
  VARIANT 2682..2685: note = Any amino acid
  VARIANT 2682..2685: note = MISC_FEATURE - This region may encompass 1-4 residues or this region may be absent in its entirety
  VARIANT 2686..2690: note = This region may encompass one of the following sequences: GQ, GN, RPG, RPGGQ, RPGGN, SSS, SKG, or SN
  VARIANT 2691..2720: note = Any amino acid
  VARIANT 2691..2720: note = MISC_FEATURE - This region may encompass 4-30 residues
  VARIANT 2722..2725: note = Any amino acid
  VARIANT 2722..2725: note = MISC_FEATURE - This region may encompass 1-4 residues or this region may be absent in its entirety
  VARIANT 2726..2730: note = This region may encompass one of the following sequences: GQ, GN, RPG, RPGGQ, RPGGN, SSS, SKG, or SN
  VARIANT 2731..2760: note = Any amino acid
  VARIANT 2731..2760: note = MISC_FEATURE - This region may encompass 4-30 residues
  VARIANT 2762..2765: note = Any amino acid
  VARIANT 2762..2765: note = MISC_FEATURE - This region may encompass 1-4 residues or this region may be absent in its entirety
  VARIANT 2766..2770: note = This region may encompass one of the following sequences: GQ, GN, RPG, RPGGQ, RPGGN, SSS, SKG, or SN
  VARIANT 2771..2800: note = Any amino acid
  VARIANT 2771..2800: note = MISC_FEATURE - This region may encompass 4-30 residues
  VARIANT 2802..2805: note = Any amino acid
  VARIANT 2802..2805: note = MISC_FEATURE - This region may encompass 1-4 residues or this region may be absent in its entirety
  VARIANT 2806..2810: note = This region may encompass one of the following sequences: GQ, GN, RPG, RPGGQ, RPGGN, SSS, SKG, or SN
  VARIANT 2811..2840: note = Any amino acid
  VARIANT 2811..2840: note = MISC_FEATURE - This region may encompass 4-30 residues
  VARIANT 2842..2845: note = Any amino acid
  VARIANT 2842..2845: note = MISC_FEATURE - This region may encompass 1-4 residues or this region may be absent in its entirety
  VARIANT 2846..2850: note = This region may encompass one of the following sequences: GQ, GN, RPG, RPGGQ, RPGGN, SSS, SKG, or SN
  VARIANT 2851..2880: note = Any amino acid
  VARIANT 2851..2880: note = MISC_FEATURE - This region may encompass 4-30 residues
  VARIANT 2882..2885: note = Any amino acid
  VARIANT 2882..2885: note = MISC_FEATURE - This region may encompass 1-4 residues or this region may be absent in its entirety
  VARIANT 2886..2890: note = This region may encompass one of the following sequences: GQ, GN, RPG, RPGGQ, RPGGN, SSS, SKG, or SN
  VARIANT 2891..2920: note = Any amino acid
  VARIANT 2891..2920: note = MISC_FEATURE - This region may encompass 4-30 residues
  VARIANT 2922..2925: note = Any amino acid
  VARIANT 2922..2925: note = MISC_FEATURE - This region may encompass 1-4 residues or this region may be absent in its entirety
  VARIANT 2926..2930: note = This region may encompass one of the following sequences: GQ, GN, RPG, RPGGQ, RPGGN, SSS, SKG, or SN
  VARIANT 2931..2960: note = Any amino acid
  VARIANT 2931..2960: note = MISC_FEATURE - This region may encompass 4-30 residues
  VARIANT 2962..2965: note = Any amino acid
  VARIANT 2962..2965: note = MISC_FEATURE - This region may encompass 1-4 residues or this region may be absent in its entirety
  VARIANT 2966..2970: note = This region may encompass one of the following sequences: GQ, GN, RPG, RPGGQ, RPGGN, SSS, SKG, or SN
  VARIANT 2971..3000: note = Any amino acid
  VARIANT 2971..3000: note = MISC_FEATURE - This region may encompass 4-30 residues
  VARIANT 3002..3005: note = Any amino acid
  VARIANT 3002..3005: note = MISC_FEATURE - This region may encompass 1-4 residues or this region may be absent in its entirety
  VARIANT 3006..3010: note = This region may encompass one of the following sequences: GQ, GN, RPG, RPGGQ, RPGGN, SSS, SKG, or SN
  VARIANT 3011..3040: note = Any amino acid
  VARIANT 3011..3040: note = MISC_FEATURE - This region may encompass 4-30 residues
  VARIANT 3042..3045: note = Any amino acid
  VARIANT 3042..3045: note = MISC_FEATURE - This region may encompass 1-4 residues or this region may be absent in its entirety
  VARIANT 3046..3050: note = This region may encompass one of the following sequences: GQ, GN, RPG, RPGGQ, RPGGN, SSS, SKG, or SN
  VARIANT 3051..3080: note = Any amino acid
  VARIANT 3051..3080: note = MISC_FEATURE - This region may encompass 4-30 residues
  VARIANT 3082..3085: note = Any amino acid
  VARIANT 3082..3085: note = MISC_FEATURE - This region may encompass 1-4 residues or this region may be absent in its entirety
  VARIANT 3086..3090: note = This region may encompass one of the following sequences: GQ, GN, RPG, RPGGQ, RPGGN, SSS, SKG, or SN
  VARIANT 3091..3120: note = Any amino acid
  VARIANT 3091..3120: note = MISC_FEATURE - This region may encompass 4-30 residues
  VARIANT 3122..3125: note = Any amino acid
  VARIANT 3122..3125: note = MISC_FEATURE - This region may encompass 1-4 residues or this region may be absent in its entirety
  VARIANT 3126..3130: note = This region may encompass one of the following sequences: GQ, GN, RPG, RPGGQ, RPGGN, SSS, SKG, or SN
  VARIANT 3131..3160: note = Any amino acid
  VARIANT 3131..3160: note = MISC_FEATURE - This region may encompass 4-30 residues
  VARIANT 3162..3165: note = Any amino acid
  VARIANT 3162..3165: note = MISC_FEATURE - This region may encompass 1-4 residues or this region may be absent in its entirety
  VARIANT 3166..3170: note = This region may encompass one of the following sequences: GQ, GN, RPG, RPGGQ, RPGGN, SSS, SKG, or SN
  VARIANT 3171..3200: note = Any amino acid
  VARIANT 3171..3200: note = MISC_FEATURE - This region may encompass 4-30 residues
  VARIANT 3202..3205: note = Any amino acid
  VARIANT 3202..3205: note = MISC_FEATURE - This region may encompass 1-4 residues or this region may be absent in its entirety
  VARIANT 3206..3210: note = This region may encompass one of the following sequences: GQ, GN, RPG, RPGGQ, RPGGN, SSS, SKG, or SN
  VARIANT 3211..3240: note = Any amino acid
  VARIANT 3211..3240: note = MISC_FEATURE - This region may encompass 4-30 residues
  VARIANT 3242..3245: note = Any amino acid
  VARIANT 3242..3245: note = MISC_FEATURE - This region may encompass 1-4 residues or this region may be absent in its entirety
  VARIANT 3246..3250: note = This region may encompass one of the following sequences: GQ, GN, RPG, RPGGQ, RPGGN, SSS, SKG, or SN
  VARIANT 3251..3280: note = Any amino acid
  VARIANT 3251..3280: note = MISC_FEATURE - This region may encompass 4-30 residues
  VARIANT 3282..3285: note = Any amino acid
  VARIANT 3282..3285: note = MISC_FEATURE - This region may encompass 1-4 residues or this region may be absent in its entirety
  VARIANT 3286..3290: note = This region may encompass one of the following sequences: GQ, GN, RPG, RPGGQ, RPGGN, SSS, SKG, or SN
  VARIANT 3291..3320: note = Any amino acid
  VARIANT 3291..3320: note = MISC_FEATURE - This region may encompass 4-30 residues
  VARIANT 3322..3325: note = Any amino acid
  VARIANT 3322..3325: note = MISC_FEATURE - This region may encompass 1-4 residues or this region may be absent in its entirety
  VARIANT 3326..3330: note = This region may encompass one of the following sequences: GQ, GN, RPG, RPGGQ, RPGGN, SSS, SKG, or SN
  VARIANT 3331..3360: note = Any amino acid
  VARIANT 3331..3360: note = MISC_FEATURE - This region may encompass 4-30 residues
  VARIANT 3362..3365: note = Any amino acid
  VARIANT 3362..3365: note = MISC_FEATURE - This region may encompass 1-4 residues or this region may be absent in its entirety
  VARIANT 3366..3370: note = This region may encompass one of the following sequences: GQ, GN, RPG, RPGGQ, RPGGN, SSS, SKG, or SN
  VARIANT 3371..3400: note = Any amino acid
  VARIANT 3371..3400: note = MISC_FEATURE - This region may encompass 4-30 residues
  VARIANT 3402..3405: note = Any amino acid
  VARIANT 3402..3405: note = MISC_FEATURE - This region may encompass 1-4 residues or this region may be absent in its entirety
  VARIANT 3406..3410: note = This region may encompass one of the following sequences: GQ, GN, RPG, RPGGQ, RPGGN, SSS, SKG, or SN
  VARIANT 3411..3440: note = Any amino acid
  VARIANT 3411..3440: note = MISC_FEATURE - This region may encompass 4-30 residues
  VARIANT 3442..3445: note = Any amino acid
  VARIANT 3442..3445: note = MISC_FEATURE - This region may encompass 1-4 residues or this region may be absent in its entirety
  VARIANT 3446..3450: note = This region may encompass one of the following sequences: GQ, GN, RPG, RPGGQ, RPGGN, SSS, SKG, or SN
  VARIANT 3451..3480: note = Any amino acid
  VARIANT 3451..3480: note = MISC_FEATURE - This region may encompass 4-30 residues
  VARIANT 3482..3485: note = Any amino acid
  VARIANT 3482..3485: note = MISC_FEATURE - This region may encompass 1-4 residues or this region may be absent in its entirety
  VARIANT 3486..3490: note = This region may encompass one of the following sequences: GQ, GN, RPG, RPGGQ, RPGGN, SSS, SKG, or SN
  VARIANT 3491..3520: note = Any amino acid
  VARIANT 3491..3520: note = MISC_FEATURE - This region may encompass 4-30 residues
  VARIANT 3522..3525: note = Any amino acid
  VARIANT 3522..3525: note = MISC_FEATURE - This region may encompass 1-4 residues or this region may be absent in its entirety
  VARIANT 3526..3530: note = This region may encompass one of the following sequences: GQ, GN, RPG, RPGGQ, RPGGN, SSS, SKG, or SN
  VARIANT 3531..3560: note = Any amino acid
  VARIANT 3531..3560: note = MISC_FEATURE - This region may encompass 4-30 residues
  VARIANT 3562..3565: note = Any amino acid
  VARIANT 3562..3565: note = MISC_FEATURE - This region may encompass 1-4 residues or this region may be absent in its entirety
  VARIANT 3566..3570: note = This region may encompass one of the following sequences: GQ, GN, RPG, RPGGQ, RPGGN, SSS, SKG, or SN
  VARIANT 3571..3600: note = Any amino acid
  VARIANT 3571..3600: note = MISC_FEATURE - This region may encompass 4-30 residues
  VARIANT 3602..3605: note = Any amino acid
  VARIANT 3602..3605: note = MISC_FEATURE - This region may encompass 1-4 residues or this region may be absent in its entirety
  VARIANT 3606..3610: note = This region may encompass one of the following sequences: GQ, GN, RPG, RPGGQ, RPGGN, SSS, SKG, or SN
  VARIANT 3611..3640: note = Any amino acid
  VARIANT 3611..3640: note = MISC_FEATURE - This region may encompass 4-30 residues
  VARIANT 3642..3645: note = Any amino acid
  VARIANT 3642..3645: note = MISC_FEATURE - This region may encompass 1-4 residues or this region may be absent in its entirety
  VARIANT 3646..3650: note = This region may encompass one of the following sequences: GQ, GN, RPG, RPGGQ, RPGGN, SSS, SKG, or SN
  VARIANT 3651..3680: note = Any amino acid
  VARIANT 3651..3680: note = MISC_FEATURE - This region may encompass 4-30 residues
  VARIANT 3682..3685: note = Any amino acid
  VARIANT 3682..3685: note = MISC_FEATURE - This region may encompass 1-4 residues or this region may be absent in its entirety
  VARIANT 3686..3690: note = This region may encompass one of the following sequences: GQ, GN, RPG, RPGGQ, RPGGN, SSS, SKG, or SN
  VARIANT 3691..3720: note = Any amino acid
  VARIANT 3691..3720: note = MISC_FEATURE - This region may encompass 4-30 residues
  VARIANT 3722..3725: note = Any amino acid
  VARIANT 3722..3725
  VARIANT 3726..3730: note = This region may encompass one of the following sequences: GQ, GN, RPG, RPGGQ, RPGGN, SSS, SKG, or SN
  VARIANT 3731..3760: note = Any amino acid
  VARIANT 3731..3760: note = MISC_FEATURE - This region may encompass 4-30 residues
  VARIANT 3762..3765: note = Any amino acid
  VARIANT 3762..3765: note = MISC_FEATURE - This region may encompass 1-4 residues or this region may be absent in its entirety
  VARIANT 3766..3770: note = This region may encompass one of the following sequences: GQ, GN, RPG, RPGGQ, RPGGN, SSS, SKG, or SN
  VARIANT 3771..3800: note = Any amino acid
  VARIANT 3771..3800: note = MISC_FEATURE - This region may encompass 4-30 residues
  VARIANT 3802..3805: note = Any amino acid
  VARIANT 3802..3805: note = MISC_FEATURE - This region may encompass 1-4 residues or this region may be absent in its entirety
  VARIANT 3806..3810: note = This region may encompass one of the following sequences: GQ, GN, RPG, RPGGQ, RPGGN, SSS, SKG, or SN
  VARIANT 3811..3840: note = Any amino acid
  VARIANT 3811..3840: note = MISC_FEATURE - This region may encompass 4-30 residues
  VARIANT 3842..3845: note = Any amino acid
  VARIANT 3842..3845: note = MISC_FEATURE - This region may encompass 1-4 residues or this region may be absent in its entirety
  VARIANT 3846..3850: note = This region may encompass one of the following sequences: GQ, GN, RPG, RPGGQ, RPGGN, SSS, SKG, or SN
  VARIANT 3851..3880: note = Any amino acid
  VARIANT 3851..3880: note = MISC_FEATURE - This region may encompass 4-30 residues
  VARIANT 3882..3885: note = Any amino acid
  VARIANT 3882..3885: note = MISC_FEATURE - This region may encompass 1-4 residues or this region may be absent in its entirety
  VARIANT 3886..3890: note = This region may encompass one of the following sequences: GQ, GN, RPG, RPGGQ, RPGGN, SSS, SKG, or SN
  VARIANT 3891..3920: note = Any amino acid
  VARIANT 3891..3920: note = MISC_FEATURE - This region may encompass 4-30 residues
  VARIANT 3922..3925: note = Any amino acid
  VARIANT 3922..3925: note = MISC_FEATURE - This region may encompass 1-4 residues or this region may be absent in its entirety
  VARIANT 3926..3930: note = This region may encompass one of the following sequences: GQ, GN, RPG, RPGGQ, RPGGN, SSS, SKG, or SN
  VARIANT 3931..3960: note = Any amino acid
  VARIANT 3931..3960: note = MISC_FEATURE - This region may encompass 4-30 residues
  VARIANT 3962..3965: note = Any amino acid
  VARIANT 3962..3965: note = MISC_FEATURE - This region may encompass 1-4 residues or this region may be absent in its entirety
  VARIANT 3966..3970: note = This region may encompass one of the following sequences: GQ, GN, RPG, RPGGQ, RPGGN, SSS, SKG, or SN
  VARIANT 3971..4000: note = Any amino acid
  VARIANT 3971..4000: note = MISC_FEATURE - This region may encompass 4-30 residues
YXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXXYXXXXXXXXXXXXXXXXXXXXXXXXXXXXXXXXXXXXXXX

(19) United States
(12) Patent Application Publication
(10) Pub. No.: US 2026/0108013 A1
(43) Pub. Date: Apr. 23, 2026

(54) Cross-Linked Elastomeric Proteins in Polar Nonaqueous Solvents and Uses Thereof

(71) Applicant: BOLT THREADS, INC., Emeryville, CA (US)

(72) Inventors: Matthew Jordan Smith, Emeryville, CA (US); Michael Eun-Suk Lee, Berkeley, CA (US); Mitchell Joseph Heinrich, Oakland, CA (US); Paul James Jehlen, Richmond, CA (US)

(21) Appl. No.: 19/172,617

(22) Filed: Apr. 7, 2025

Related U.S. Application Data
(63) Continuation of application No. 16/515,577, filed on Jul. 18, 2019, now Pat. No. 12,268,275.
(60) Provisional application No. 62/700,197, filed on Jul. 18, 2018.

(51) Int. Cl. A43B 13/04; A43B 3/10; A43B 3/12; A43B 13/02; A43B 13/12; A43B 13/14; A43B 13/16; A43B 13/18; A43B 17/00; A43B 23/08; B29C 39/00; B29D 35/12; B29L 31/50; C07K 1/02; C07K 1/14; C07K 14/435; C07K 14/78; C08J 3/24; C08J 9/00; C08J 9/04; C08J 9/08; C08J 9/12

(52) U.S. Cl. CPC A43B 13/04; A43B 3/101; A43B 3/108; A43B 3/128; A43B 13/02; A43B 13/122; A43B 13/125; A43B 13/127; A43B 13/14; A43B 13/16; A43B 13/186; A43B 13/187; A43B 13/188; A43B 17/003; A43B 23/088; B29C 39/003; B29D 35/122; C07K 1/02; C07K 1/14; C07K 14/43563; C07K 14/78; C08J 3/247; C08J 9/0066; C08J 9/04; C08J 9/08; C08J 9/122; B29K 2089/00; B29L 2031/504; B29L 2031/507; C08J 2389/00

(57) ABSTRACT

Disclosed herein are improved cross-linked resilin compositions and methods of making these improved compositions. These include new methods of cross-linking resilin compositions, use of polar nonaqueous solvents for adjusting material properties of cross-linked resilin solid compositions, and resilin foam compositions and methods of making the same.

### CROSS-REFERENCE TO RELATED APPLICATIONS

This is a Continuation of U.S. patent application Ser. No. 16/515,577, filed Jul. 18, 2019, which claims the benefit of U.S. Provisional Application No. 62/700,197, filed Jul. 18, 2018, the contents of which are incorporated by reference in their entirety.

### INCORPORATION BY REFERENCE FOR MATERIAL SUBMITTED ELECTRONICALLY

The Sequence Listing, which is a part of the present disclosure, is submitted concurrently with the specification as an XML file. The name of the XML file containing the Sequence Listing is “50027A_SeqListing.xml”, which was created on Dec. 24, 2025, and is 514,664 bytes in size. The subject matter of the Sequence Listing is incorporated herein in its entirety by reference.

### BACKGROUND

Elastomeric proteins are polypeptides that exhibit viscoelastic mechanical properties, and include elastin, resilin, abductin, and octopus arterial elastomers. Resilin is a particularly interesting elastomeric protein because it dissipates very little energy during loading and unloading. Resilin is found in many insects, and the low energy dissipation enables the extraordinary ability of many insect species to jump or pivot their wings very efficiently. The unique properties of resilin make it an interesting elastomeric material that could have many industrial applications. What is needed, therefore, are new resilin compositions and methods of making the same that have desirable mechanical properties and are suitable for large-scale, efficient production.

Variations of natural resilins and resilin-like proteins (based on resilin sequences) have been recombinantly produced by a number of groups in E. coli cultures, and have been isolated by lysing the cells to extract recombinantly expressed proteins, and using affinity chromatography techniques to purify (Elvin et al., 2005; Charati et al.; 2009, McGann et al., 2013). The recombinantly produced resilin and resilin-like proteins have been cross-linked targeting the tyrosine residues that also form the cross-links in natural resilin (see, for example, Elvin et al., 2005; Qin et al., 2011). Recombinantly produced resilin has also been cross-linked targeting lysine residues (Li et al., 2011) or cysteine residues (McGann et al., 2013). Cross-linking methods have included enzymatically catalyzed cross-linking and photoactivated cross-linking. Enzymes for enzymatic catalysis of cross-linking can be hard to remove from the final composition, while photoactivated cross-linking may suffer from inefficiency due to only the surface being exposed to light, while also leaving impurities. The enzymatic impurities can degrade the resilin, while impurities from either enzymes or photocatalysts left in the final solid cross-linked composition can result in undesirable mechanical properties. What is needed, therefore, are new methods of cross-linking recombinant resilin proteins that do not leave behind degradation causing impurities and can be performed efficiently at a large scale.

### SUMMARY OF THE INVENTION

Disclosed herein are improved cross-linked resilin compositions and methods of making these improved compositions. These include new methods of cross-linking resilin compositions and the use of polar nonaqueous solvents for adjusting material properties of cross-linked resilin solid compositions.

In some embodiments, provided herein is a method of cross-linking recombinant resilin, comprising: providing a composition comprising purified recombinant resilin; placing said recombinant resilin in a cross-linking solution comprising ammonium persulfate; and incubating said recombinant resilin in said cross-linking solution at a temperature of at least 60° C., thereby generating a cross-linked recombinant resilin solid composition.

In some embodiments, the incubation is performed for at least 15 minutes, at least 30 minutes, at least 45 minutes, at least 60 minutes, at least 90 minutes, or at least 2 hours.

In some embodiments, the recombinant resilin in said cross-linking solution is incubated at a temperature of from 60° C. to 85° C., from 70° C. to 85° C., or from 75° C. to 85° C.

In some embodiments, the incubation is performed for at least 2 hours.

In some embodiments, the cross-linking solution does not comprise a photocatalyst or a cross-linking enzyme.

In some embodiments, the cross-linked recombinant resilin solid composition is stable at room temperature for more than 5 days, more than 10 days, more than 20 days, or more than 40 days.

In some embodiments, the purified recombinant resilin is prepared by recombinantly expressing a gene encoding said recombinant resilin in a modified organism in a culture, and purifying expressed recombinant resilin from said culture.

Also provided herein, according to some embodiments, is a composition comprising cross-linked recombinant resilin, wherein said recombinant resilin has been cross-linked by exposure of said resilin to ammonium persulfate and heat. In some embodiments, the composition does not comprise a cross-linking enzyme. In some embodiments, the composition does not comprise a photocatalyst.

In some embodiments, provided herein is a recombinant resilin composition comprising a cross-linked recombinant resilin in a polar nonaqueous solvent.

In some embodiments, the polar nonaqueous solvent is a protic solvent. In some embodiments, the protic solvent is selected from the group consisting of: glycerol, propylene glycol, and ethylene glycol. In some embodiments, the polar nonaqueous solvent is an aprotic solvent. In some embodiments, the aprotic solvent is DMSO.

In some embodiments, the composition comprises from 20-40% resilin by weight.

In some embodiments, the resilin is at least 20%, at least 50%, at least 70%, or at least 80% full length resilin as a portion of all resilin as measured by size exclusion chromatography.

In some embodiments, the polar nonaqueous solvent is at least 60%, at least 70%, at least 80%, at least 90%, at least 95%, at least 98%, or at least 99% by volume of said solvent.

In some embodiments, the recombinant resilin composition has an elastic modulus greater than a similar cross-linked recombinant resilin in an aqueous solvent.

In some embodiments, the recombinant resilin composition comprises a hardness of at least 10 as measured using a Shore OO Durometer via ASTM D2240.

In some embodiments, the recombinant resilin composition comprises a hardness of from about 10 to about 50 as measured using a Shore OO Durometer via ASTM D2240.

In some embodiments, the recombinant resilin composition comprises a rebound resilience from about 40% to about 60% as measured by ASTM D7121.

In some embodiments, the recombinant resilin composition comprises a compressive stress at 25% of about 6 to about 8 psi as measured by ASTM D575.

In some embodiments, the recombinant resilin composition does not undergo an elastic to plastic transition below 2 kN of compressive force as measured by a Zwick compression test. In some embodiments, the recombinant resilin composition is a foam material.

Also provided herein, according to some embodiments, is a method of preparing a recombinant resilin solid, comprising: providing a cross-linked recombinant resilin solid composition in an aqueous solvent; and exchanging said aqueous solvent with a polar nonaqueous solvent.

In some embodiments, the polar nonaqueous solvent is a protic solvent. In some embodiments, the protic solvent is selected from the group consisting of: glycerol, propylene glycol, and ethylene glycol. In some embodiments, the polar nonaqueous solvent is an aprotic solvent. In some embodiments, the aprotic solvent is DMSO.

In some embodiments, the solvent exchange is performed for at least 8 hours, at least 16 hours, at least 24 hours, or at least 48 hours.

In some embodiments, the solvent exchange is performed at about 60° C.

In some embodiments, the cross-linked recombinant resilin solid composition comprises at least 20%, at least 50%, at least 70%, or at least 80% full length resilin as a portion of all resilin as measured by size exclusion chromatography.

In some embodiments, the cross-linked recombinant solid composition is prepared by the ammonium persulfate and heat method described herein.

Also provided herein, according to some embodiments, is a method of tuning material properties of a solid composition comprising cross-linked recombinant resilin solid, comprising: providing a cross-linked recombinant resilin solid composition comprising an aqueous solvent; and performing a solvent exchange to replace said aqueous solvent with a polar nonaqueous solvent. In some embodiments, replacing said solvent changes the elastic modulus, the resilience, the hardness, the maximum elastic compressive load, or the material lifetime of the cross-linked recombinant resilin solid composition.

Also provided herein, according to some embodiments, is a method of preparing a recombinant cross-linked resilin solid, comprising: providing a composition comprising purified recombinant resilin; placing said recombinant resilin in an aqueous solvent comprising ammonium persulfate; incubating said recombinant resilin in said aqueous solvent at a temperature of at least 60° C., thereby generating a cross-linked recombinant resilin solid composition; and exchanging said aqueous solvent with a polar nonaqueous solvent selected from the group consisting of: glycerol, propylene glycol, ethylene glycol, and DMSO.

Also provided herein, according to some embodiments, is a method of preparing a recombinant resilin foam, comprising: providing a cross-linked recombinant resilin solid composition in an aqueous solvent; exchanging said aqueous solvent with a polar nonaqueous solvent; and introducing one or more bubbles to the cross-linked recombinant resilin solid composition.

In some embodiments, the polar nonaqueous solvent is a protic solvent. In some embodiments, the protic solvent is selected from the group consisting of: glycerol, propylene glycol, and ethylene glycol. In some embodiments, the polar nonaqueous solvent is an aprotic solvent. In some embodiments, the aprotic solvent is DMSO.

In some embodiments, the solvent exchange is performed for at least 8 hours, at least 16 hours, at least 24 hours, or at least 48 hours.

In some embodiments, the solvent exchange is performed at about 60° C.

In some embodiments, the cross-linked recombinant resilin solid composition comprises at least 20%, at least 50%, at least 70%, or at least 80% full length resilin as a portion of all resilin as measured by size exclusion chromatography.

In some embodiments, the cross-linked recombinant solid composition is prepared by the ammonium persulfate and heat method described herein.

In some embodiments, the introducing comprises adding a blowing agent to the cross-linked recombinant resilin solid composition.

In some embodiments, the blowing agent comprises a chemical blowing agent. In some embodiments, the chemical blowing agent comprises sodium bicarbonate, potassium bicarbonate, ammonium, azodicarbonamide, isocyanate, hydrazine, isopropanol, 5-phenyltetrazole, triazole, 4,4′oxybis(benzenesulfonyl hydrazide) (OBSH), trihydrazine triazine (THT), hydrogen phosphate, tartaric acid, citric acid, and toluenesulphonyl semicarbazide (TSS). In some embodiments, the chemical blowing agent comprises sodium bicarbonate.

In some embodiments, the blowing agent comprises a physical blowing agent. In some embodiments, the physical blowing agent comprises chlorofluorocarbon (CFC), dissolved nitrogen, N₂, CH₄, H₂, CO₂, Ar, pentane, isopentane, hexane, methylene dichloride, and dichlorotetra-fluoroethane. In some embodiments, the physical blowing agent comprises dissolved nitrogen.

According to some embodiments, provided herein is a method for producing a composition comprising a recombinant resilin protein, the method comprising: culturing a population of recombinant host cells in a fermentation, wherein said recombinant host cells comprise a vector comprising a secreted resilin coding sequence, and wherein said recombinant host cells secrete a recombinant resilin protein encoded by said secreted resilin coding sequence; and purifying said recombinant resilin protein from said fermentation.

In some embodiments, the recombinant resilin protein is a full-length or truncated native resilin. In some embodiments, the native resilin is from an organism selected from the group consisting of: Drosophila sechellia, Acromyrmex echinatior, Aeshna, Haematobia irritans, Ctenocephalides felis, Bombus terrestris, Tribolium castaneum, Apis mellifera, Nasonia vitripennis, Pediculus humanus corporis, Anopheles gambiae, Glossina morsitans, Atta cephalotes, Anopheles darlingi, Acyrthosiphon pisum, Drosophila virilis, Drosophila erecta, Lutzomyia longipalpis, Rhodnius prolixus, Solenopsis invicta, Culex quinquefasciatus, Bactrocera cucurbitae, and Trichogramma pretiosum. In some embodiments, the recombinant resilin protein comprises SEQ ID NO: 1. In some embodiments, the recombinant resilin protein comprises SEQ ID NO: 4.

In some embodiments, the recombinant resilin protein comprises an alpha mating factor secretion signal. In some embodiments, the recombinant resilin protein comprises a FLAG-tag. In some embodiments, the vector comprises more than one secreted resilin coding sequence.

In some embodiments, the recombinant host cells are yeast cells. In some embodiments, the yeast cells are methylotrophic yeast cells. In some embodiments, the recombinant host cells are a species selected from the group consisting of: Pichia (Komagataella) pastoris, Hansenula polymorpha, Arxula adeninivorans, Yarrowia lipolytica, Pichia (Scheffersomyces) stipitis, Pichia methanolica, Saccharomyces cerevisiae, and Kluyveromyces lactis.

In some embodiments, the recombinant host cells produce the recombinant resilin at a rate of greater than 2 mg resilin/g dry cell weight/hour. In some embodiments, the recombinant host cells produce a secreted fraction of the recombinant resilin that is greater than 50% as compared to the total recombinant resilin protein expressed by the recombinant host cells. In some embodiments, the recombinant host cells secrete the recombinant resilin at a rate of greater than 2 mg resilin/g dry cell weight/hour. In some embodiments, greater than 80% of the recombinant resilin is outside of the recombinant host cells in said fermentation. In some embodiments, the fermentation comprises at least 2 g recombinant resilin/L.

In some embodiments, purifying said recombinant resilin protein comprises: generating a first pellet fraction and a first supernatant fraction by centrifuging the fermentation; and isolating the recombinant resilin protein from the first pellet fraction. In some embodiments, purifying said recombinant resilin protein further comprises: adding a chaotrope to the first pellet fraction to generate a solution in which the recombinant resilin protein is soluble; generating a second supernatant fraction and a second pellet fraction by centrifuging the first pellet fraction comprising said chaotrope; and isolating the soluble full-length resilin from the second supernatant fraction.

In some embodiments, provided herein is a vector comprising a secreted resilin coding sequence. In some embodiments, the secreted resilin coding sequence encodes a full-length or truncated native resilin. In some embodiments, the secreted resilin coding sequence encodes a modified full-length or truncated native resilin. In some embodiments, the modified resilin comprises an addition, subtraction, replacement, or change in position of an amino acid residue capable of cross-linking to another resilin.

In some embodiments, the full-length or truncated native resilin is from an organism selected from the group consisting of: Drosophila sechellia, Acromyrmex echinatior, Aeshna, Haematobia irritans, Ctenocephalides felis, Bombus terrestris, Tribolium castaneum, Apis mellifera, Nasonia vitripennis, Pediculus humanus corporis, Anopheles gambiae, Glossina morsitans, Atta cephalotes, Anopheles darlingi, Acyrthosiphon pisum, Drosophila virilis, Drosophila erecta, Lutzomyia longipalpis, Rhodnius prolixus, Solenopsis invicta, Culex quinquefasciatus, Bactrocera cucurbitae, and Trichogramma pretiosum.

In some embodiments, the secreted resilin coding sequences encodes a polypeptide comprising SEQ ID NO: 1. In some embodiments, the secreted resilin coding sequence encodes a polypeptide comprising SEQ ID NO: 4. In some embodiments, the secreted resilin coding sequence encodes a recombinant resilin comprising one or more A-repeats or quasi-A-repeats. In some embodiments, the secreted resilin coding sequence encodes a recombinant resilin comprising one or more B-repeats or quasi-B-repeats. In some embodiments, the secreted resilin coding sequence encodes a recombinant resilin comprising either one or more A-repeats or quasi-A-repeats or one or more B-repeats or quasi-B repeats but not both. In some embodiments, the secreted resilin coding sequence encodes a recombinant resilin comprising one or more A-repeats or quasi-A-repeats and one or more B-repeats or quasi-B-repeats.

In some embodiments, the recombinant resilin further comprises a chitin binding domain. In some embodiments, the secreted resilin coding sequence encodes a polypeptide comprising an alpha mating factor secretion signal. In some embodiments, the secreted resilin coding sequence comprises a FLAG-tag.

In some embodiments, the vector comprises more than one secreted resilin coding sequence. In some embodiments, the vector comprises 3 secreted resilin coding sequences. In some embodiments, the secreted resilin coding sequence is operatively linked to a constitutive or inducible promoter.

Also provided herein, according to some embodiments, is a recombinant host cell comprising one or more vectors comprising a secreted resilin coding sequence. In some embodiments, the recombinant host cell is a yeast cell. In some embodiments, the yeast cell is a methylotrophic yeast cell. In some embodiments, the recombinant host cell is a species selected from the group consisting of: Pichia (Komagataella) pastoris, Hansenula polymorpha, Arxula adeninivorans, Yarrowia lipolytica, Pichia (Scheffersomyces) stipitis, Pichia methanolica, Saccharomyces cerevisiae, and Kluyveromyces lactis.

In some embodiments, the recombinant host cell comprises 3 vectors comprising a secreted resilin coding sequence.

In some embodiments, the recombinant host cell produces recombinant resilin at a rate of greater than 2 mg resilin/g dry cell weight/hour. In some embodiments, the recombinant host cell has a secreted fraction of recombinant resilin that is greater than 50%. In some embodiments, the recombinant host cell secretes resilin at a rate of greater than 2 mg resilin/g dry cell weight/hour.

Also provided herein, according to some embodiments, is a fermentation comprising a recombinant host cell comprising one or more vectors comprising a secreted resilin coding sequence and a culture medium suitable for growing the recombinant host cell.

In some embodiments, the fermentation comprises at least 2 g recombinant resilin/L.

In some embodiments of the fermentation, greater than 80% of recombinant resilin is outside of the recombinant host cells.

In some embodiments of the fermentation, the recombinant resilin is full-length recombinant resilin.

Also provided herein, according to some embodiments, is a composition comprising recombinant resilin derived from a fermentation comprising a recombinant host cell comprising one or more vectors comprising a secreted resilin coding sequence and a culture medium suitable for growing the recombinant host cell. In some embodiments, the composition comprises at least 60% by weight of recombinant resilin.

In some embodiments, the composition has similar properties compared to compositions comprising similar amounts of native resilins. In some embodiments, the composition has different properties compared to compositions comprising similar amounts of native resilins.

In some embodiments, the composition comprises a resilience of greater than 50%. In some embodiments, the composition comprises has a compressive elastic modulus of less than 10 MPa. In some embodiments, the composition has a tensile elastic modulus of less than 10 MPa. In some embodiments, the composition has a shear modulus of less than 1 MPa. In some embodiments, the composition has an extension to break of greater than 1%. In some embodiments, the composition has a maximum tensile strength of greater than 0.1 kPa. In some embodiments, the composition has a Shore OO Hardness of less than 90. In some embodiments, the composition comprises full-length resilin.

Also provided herein, according to some embodiments, is a method for producing a composition comprising recombinant resilin, the method comprising the step of culturing a recombinant host cell comprising one or more vectors comprising a secreted resilin coding sequence to produce a fermentation under conditions that promote secretion of recombinant resilin from the recombinant host cell.

In some embodiments, the method for producing a composition comprising recombinant resilin further comprises the step of purifying said recombinant resilin to produce full-length native resilin. In some embodiments, purifying said recombinant resilin to produce full-length native resilin comprises: generating a first pellet fraction and a first supernatant fraction by centrifuging the fermentation; and isolating the recombinant resilin protein from the first pellet fraction. In some embodiments, isolating the recombinant resilin protein from the first pellet fraction comprises: adding a chaotrope to the first pellet fraction to generate a solution in which the recombinant resilin protein is soluble; generating a second supernatant fraction and a second pellet fraction by centrifuging the first pellet fraction comprising said chaotrope; and isolating the recombinant resilin protein from the second supernatant fraction.

In some embodiments, the method for producing a composition comprising recombinant resilin further comprises the step of cross-linking a plurality of said recombinant resilins. In some embodiments, said cross-linking is enzymatic cross-linking. In some embodiments, said cross-linking is photochemical cross-linking. In some embodiments, the recombinant resilin protein comprises a full-length resilin protein.

Also provided herein, according to some embodiments, is a fermentation comprising a culture medium and a recombinant host cell, wherein the recombinant host cell comprises a vector, wherein the vector comprises a secreted resilin coding sequence, and wherein the recombinant host cell secretes recombinant resilin at a rate of at least 2 mg/g dry cell weight/hour.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead placed upon illustrating the principles of various embodiments of the invention.

FIG. 1 schematically illustrates the structure of an exemplary resilin.

FIG. 2 is a flow diagram of methods for the production of compositions comprising recombinant resilins.

FIG. 3 is an illustrative map of a vector that comprises 3 secreted resilin coding sequences.

FIG. 4 shows photographs of proteinaceous block co-polymers comprising cross-linked purified recombinant resilins in various shapes and forms.

FIG. 5 shows photographs of the compression of a proteinaceous block co-polymer comprising cross-linked recombinant resilin.

FIG. 6 shows the total degraded resilin as measured by ELISA using an antibody specific for the FLAG tag on resilin from resilin solid compositions cross-linked via ammonium persulfate plus heat (AP); ammonium persulfate plus heat and washed 3 times to remove excess cross-linking solution (AP 3× washed); and enzymatic cross-linking via horseradish peroxidase (HRP).

FIG. 7 shows a photograph of resilin foam with 10 wt % fumed silica (left) and 5 wt % fumed silica (right).

FIG. 8 shows a photograph of resilin foam with 5 wt % fumed silica. A quarter dollar is included for comparison.

FIG. 9 shows an image of glycerol-based and water-based cross-linked resilin compositions after cross-linking (top) and after 7 days of unsealed storage for one week (bottom).

FIGS. 10A, 10B, and 10C depict stress strain curves as measured by a rheometer for the following cross-linked resilin compositions: 27% by weight resilin in a glycerol solvent prepared by a 60% glycerol solvent exchange followed by a 100% glycerol solvent exchange (FIG. 10A); 27% by weight resilin in a propylene glycol solvent (FIG. 10B); and 27% full length resilin (i.e., about 80% full length resilin as a proportion of all resilin) in a propylene glycol solvent (FIG. 10C).

FIG. 11 shows a stress-strain curve of resilin foam with 5 wt % fumed silica.

FIG. 12 shows a plot of the relative values of resilience and elastic modulus as measured by a rheometer for each of the following cross-linked resilin solid compositions: 20 wt % resilin, propylene glycol (“A”); 27 wt % resilin, propylene glycol (“B”); 40 wt % resilin, propylene glycol (“C”); resilin coacervate, propylene glycol (“D”); 27 wt % full-length resilin, propylene glycol (“E”); 27 wt % resilin, 60-100% glycerol (“F”); 27 wt % resilin, 100% glycerol (“H”); and 27 wt % resilin, ethylene glycol (“I”).

FIG. 13 shows a plot of applied force vs. strain curves as measured from the Zwick tensile tester for each of the following cross-linked resilin solids: 27 wt % resilin ethylene glycol solid (“A”), 27 wt % resilin propylene glycol solid (“C), coacervate resilin propylene glycol solid (“D”), 27 wt % resilin 60-100% glycerol solid (“G”), and 27 wt % resilin 100% glycerol solid (“H”).

The figures depict various embodiments of the present disclosure for purposes of illustration only. One skilled in the art will readily recognize from the following discussion that alternative embodiments of the structures and methods illustrated herein may be employed without departing from the principles described herein.

### DETAILED DESCRIPTION

The details of various embodiments of the invention are set forth in the description below. Other features, objects, and advantages of the invention will be apparent from the description and the drawings, and from the claims.

### Definitions

Unless defined otherwise, all technical and scientific terms used herein have the same meaning as is commonly understood by one of ordinary skill in the art to which this disclosure pertains.

The terms “a” and “an” and “the” and similar referents as used herein refer to both the singular and the plural, unless otherwise indicated herein or clearly contradicted by context.

The term “about,” “approximately,” or “similar to” means within an acceptable error range for the particular value as determined by one of ordinary skill in the art, which can depend in part on how the value is measured or determined, or on the limitations of the measurement system. It should be understood that all ranges and quantities described below are approximations and are not intended to limit the invention. Where ranges and numbers are used these can be approximate to include statistical ranges or measurement errors or variation. In some embodiments, for instance, measurements could be plus or minus 10%.

Amino acids can be referred to by their single-letter codes or by their three-letter codes. The single-letter codes, amino acid names, and three-letter codes are as follows: G-Glycine (Gly), P—Proline (Pro), A—Alanine (Ala), V—Valine (Val), L—Leucine (Leu), I—Isoleucine (Ile), M—Methionine (Met), C—Cysteine (Cys), F—Phenylalanine (Phe), Y—Tyrosine (Tyr), W—Tryptophan (Trp), H—Histidine (His), K—Lysine (Lys), R—Arginine (Arg), Q—Glutamine (Gln), N—Asparagine (Asn), E—Glutamic Acid (Glu), D—Aspartic Acid (Asp), S—Serine (Ser), T—Threonine (Thr).

The terms “including,” “includes,” “having,” “has,” “with,” or variants thereof are intended to be inclusive in a manner similar to the term “comprising.”

The term “microbe” as used herein refers to a microorganism, and refers to a unicellular organism. As used herein, the term includes all bacteria, all archaea, unicellular protista, unicellular animals, unicellular plants, unicellular fungi, unicellular algae, all protozoa, and all chromista.

The term “native” as used herein refers to compositions found in nature in their natural, unmodified state.

The terms “optional” or “optionally” mean that the feature or structure may or may not be present, or that an event or circumstance may or may not occur, and that the description includes instances where a particular feature or structure is present and instances where the feature or structure is absent, or instances where the event or circumstance occurs and instances where the event or circumstance does not occur.

The term “secreted fraction” as used herein refers to the fraction of recombinant resilins that are secreted from cells compared to the total resilins produced by the cells.

The term “secretion signal” as used herein refers to a short peptide that when fused to a polypeptide mediates the secretion of that polypeptide from a cell.

The term “secreted resilin coding sequence” as used herein refers to a nucleotide sequence that encodes a resilin as provided herein fused at its N-terminus to a secretion signal and optionally at its C-terminus to a tag peptide or polypeptide.

The term “recombinant” as used herein in reference to a polypeptide (e.g., resilin) refers to a polypeptide that is produced in a recombinant host cell, or to a polypeptide that is synthesized from a recombinant nucleic acid.

The term “recombinant host cell” as used herein refers to a host cell that comprises a recombinant nucleic acid.

The term “recombinant nucleic acid” as used herein refers to a nucleic acid that is removed from its naturally occurring environment, or a nucleic acid that is not associated with all or a portion of a nucleic acid abutting or proximal to the nucleic acid when it is found in nature, or a nucleic acid that is operatively linked to a nucleic acid that it is not linked to in nature, or a nucleic acid that does not occur in nature, or a nucleic acid that contains a modification that is not found in that nucleic acid in nature (e.g., insertion, deletion, or point mutation introduced artificially, e.g., by human intervention), or a nucleic acid that is integrated into a chromosome at a heterologous site. The term includes cloned DNA isolates and nucleic acids that comprise chemically-synthesized nucleotide analog.

The term “vector” as used herein refers to a nucleic acid molecule capable of transporting another nucleic acid to which it has been linked. One type of vector is a “plasmid,” which generally refers to a circular double stranded DNA loop into which additional DNA segments can be ligated, but also includes linear double-stranded molecules such as those resulting from amplification by the polymerase chain reaction (PCR) or from treatment of a circular plasmid with a restriction enzyme. Other vectors include bacteriophages, cosmids, bacterial artificial chromosomes (BAC), and yeast artificial chromosomes (YAC). Another type of vector is a viral vector, wherein additional DNA segments can be ligated into the viral genome. Certain vectors are capable of autonomous replication in a cell into which they are introduced (e.g., vectors having an origin of replication that functions in the cell). Other vectors can be integrated into the genome of a cell upon introduction into the cell, and are thereby replicated along with the cell genome.

The term “repeat” as used herein, in reference to an amino acid or nucleic acid sequence, refers to a sub-sequence that is present more than once in a polynucleotide or polypeptide (e.g., a concatenated sequence). A polynucleotide or polypeptide can have a direct repetition of the repeat sequence without any intervening sequence, or can have non-consecutive repetition of the repeat sequence with intervening sequences. The term “quasi-repeat” as used herein, in reference to amino acid or nucleic acid sequences, is a sub-sequence that is inexactly repeated (i.e., wherein some portion of the quasi-repeat subsequence is variable between quasi-repeats) across a polynucleotide or polypeptide. Repeating polypeptides and DNA molecules (or portions of polypeptides or DNA molecules) can be made up of either repeat sub-sequences (i.e., exact repeats) or quasi-repeat sub-sequences (i.e., inexact repeats).

The term “native resilin” as used herein refers to an elastomeric polypeptide or protein produced by insects. GenBank Accession Nos. of non-limiting examples of native resilin includes the following NCBI sequence numbers: NP 995860 (Drosophila melanogaster), NP 611157 (Drosophila melanogaster), Q9V7U0 (Drosophila melanogaster), AAS64829, AAF57953 (Drosophila melanogaster), XP 001817028 (Tribolium castaneum) and XP001947408 (Acyrthosiphon pisum).

The term “modified” as used herein refers to a protein or polypeptide sequence that differs in composition from a native protein or polypeptide sequence, where the functional properties are preserved to within 10% of the native protein or polypeptide properties. In some embodiments, the difference between the modified protein or polypeptide and the native protein or polypeptide can be in primary sequence (e.g., one or more amino acids are removed, inserted or substituted) or post-translation modifications (e.g., glycosylation, phosphorylation). Amino acid deletion refers to removal of one or more amino acids from a protein. Amino acid insertion refers to one or more amino acid residues being introduced in a protein or polypeptide. Amino acid insertions may comprise N-terminal and/or C-terminal fusions as well as intra-sequence insertions of single or multiple amino acids. Amino acid substitution includes non-conservative or conservative substitution, where conservative amino acid substitution tables are well known in the art (see for example Creighton (1984) Proteins. W. H. Freeman and Company (Eds)). In some embodiments, the modified protein or polypeptide and the native protein or polypeptide amino acid or nucleotide sequence identity is at least 60%, at least 65%, at least 70%, at least 75%, at least 80%, at least 85%, at least 90%, at least 95%, or at least 98% of the amino acids or nucleotide bases.

The term “truncated” as used herein refers to a protein or polypeptide sequence that is shorter in length than a native protein or polypeptide. In some embodiments, the truncated protein or polypeptide can be greater than 10%, or greater than 20%, or greater than 30%, or greater than 40%, or greater than 50%, or greater than 60%, or greater than 70%, or greater than 80%, or greater than 90% of the length of the native protein or polypeptide.

The term “homolog” or “substantial similarity,” as used herein, when referring to a polypeptide, nucleic acid or fragment thereof, indicates that, when optimally aligned with appropriate amino acid or nucleotide insertions or deletions with another amino acid or nucleic acid (or its complementary strand), there is amino acid or nucleotide sequence identity in at least 60%, at least 65%, at least 70%, at least 75%, at least 80%, at least 85%, at least 90%, at least 95%, or at least 98% of the amino acids or nucleotide bases, as measured by any well-known algorithm of sequence identity, such as FASTA, BLAST or Gap, as discussed above.

The term “resilin” as used herein refers to a protein or a polypeptide, capable of cross-linking to form an elastomer, where the protein or polypeptide is a native resilin, or a native resilin that is modified, or a native resilin that is truncated. Resilins of the present invention are preferably recombinant resilins. In some embodiments, recombinant resilins comprise a natural or modified (e.g., truncated or concatenated) nucleotide sequence coding for resilin or resilin fragments (e.g., isolated from insects), heterologously expressed and secreted from a host cell. In preferred embodiments, the secreted recombinant resilin protein is collected from a solution extracellular to the host cell. In some embodiments, resilin is in a form of a foam material, e.g., a solid foam.

As used herein, the term “elastomer” refers to a polymer with viscoelasticity and typically weak inter-molecular forces (except for covalent cross-links between molecules, if they are present). Viscoelasticity is a property of materials that exhibit both viscous and elastic characteristics when undergoing deformation, and therefore exhibit time-dependent strain. Elasticity is associated with bond stretching along crystallographic planes in an ordered solid, and viscosity is the result of the diffusion of atoms or molecules inside an amorphous material. Elastomers that are viscoelastic, therefore, generally have low Young's modulus and high failure strain compared with other materials. Due to the viscous component of the material, viscoelastic materials dissipate energy when a load is applied and then removed. This phenomenon is observed as hysteresis in the stress-strain curve of viscoelastic materials. As a load is applied there is a particular stress-strain curve, and as the load is removed the stress-strain curve upon unloading is different than that of the curve during loading. The energy dissipated is the area between the loading and unloading curves.

As used herein, the term “nonaqueous” refers to a solvent that predominantly comprises one or more compounds that are not water. This includes compositions that have undergone a solvent exchange process with a solvent that results in an overall decrease in the proportion of water present as a solvent, i.e., water has been replaced by non-water molecules as a solvent. In some embodiments, a nonaqueous solvent is one that comprises less than 50% water. A polar nonaqueous solvent, as used herein with respect to solvents for cross-linked resilin compositions, refers to any nonaqueous solvent that is capable of dissolving resilin.

Recitation of ranges of values herein are merely intended to serve as a shorthand method of referring individually to each separate value inclusively falling within the range, unless otherwise indicated herein, and each separate value is incorporated into the specification as if it were individually recited herein.

### Resilin Compositions

Provided herein are compositions comprising recombinant resilins, and methods for their production.

Resilins have many unique properties compared to petroleum-based elastomers. Most notably, resilin has an extreme elastic efficiency (i.e., resilience), where very little of the energy input into deformation is lost as heat. Other desirable properties of resilin include, for example, compressive elastic modulus, tensile elastic modulus, shear modulus, hardness, rebound, and compression set. Moreover, resilin is a protein, and therefore can be biodegraded, which makes it more environmentally friendly than petroleum-based polymers. Also, resilin is biocompatible and can therefore be used in applications that involve contact with humans or animals. Lastly, the mechanical properties of recombinant resilins can be tuned through varying protein sequence, protein structure, amount of intermolecular cross-linking and processing variables to produce elastomers designed for a universe of specific applications.

In some embodiments, provided herein are cross-linked resilin compositions with desirable mechanical properties and methods of producing them. In some embodiments, provided herein are methods of cross-linking resilin compositions to form a cross-linked resilin solid that can be performed efficiently in large batches and is not susceptible to degradation from impurities left over from the cross-linking reaction. In some embodiments, the cross-linking reaction comprises exposure of the resilin to a persulfate, such as ammonium persulfate. Heat can be applied to initiate a cross-linking reaction catalyzed by persulfate. This type of cross-linking reaction does not leave any photoactive or enzymatic compounds in the composition. Furthermore, this cross-linking reaction does not require photoactivation, so large batches can be produced efficiently without the requirement for light to reach all parts of the cross-linking solution. In some embodiments, cross-linking occurs in vessels or molds such that the recombinant resilin compositions obtained have specific shapes or forms.

The cross-linked resilin solid compositions provided herein also include cross-linked resilin compositions comprising a polar nonaqueous solvent to provide desired mechanical properties, such as elastic modulus, hardness, maximum elastic compressive load, resilience, and material lifetime/fatigue that are preferred in certain applications. In some embodiments, the compositions are made by performing a solvent exchange with a resilin composition to replace an aqueous solvent with a nonaqueous solvent. Solvents that are capable of doing solvent exchange with cross-linked resilin include solvents that dissolve resilin in its non-crosslinked form.

In preferred embodiments, the nonaqueous solvent is non-volatile and water soluble or polar. In some embodiments, the molecular weight of the solvent is about 100 or less. In some embodiments, the polar nonaqueous solvent comprises glycerol, propylene glycol, ethylene glycol, or DMSO. In some embodiments, the solvent exchange is done using a polar nonaqueous solvent gradient (e.g., from 60% to 100% glycerol).

In some embodiments, the cross-linked resilin compositions described herein can be used to provide a composition having improved physical properties, including, e.g., for absorption of energy from an applied force as desired. In some embodiments, the cross-linked resilin compositions described herein can be used to improve existing products containing rubber. In particular, some of the cross-linked resilin compositions provided herein can absorb a large amount of force, while not transitioning to an inelastic material.

In some embodiments, the cross-linked resilin compositions provided herein can be used as a mid-sole. In some embodiments, the cross-linked resilin compositions provided herein can be used as part of a core for a golf ball. In other embodiments, the cross-linked resilin compositions provided herein can be used in handles or grips, e.g., for sports equipment such as golf clubs or tennis rackets, as bicycle grips or motorcycle grips, or as groups for tools and industrial uses such as hammers, nail guns, jackhammers, and any other tools where it is preferable to absorb and return energy. In some embodiments, the cross-linked resilin compositions provided herein can be used in brushings or dampenings, e.g., skate board trucks or hard drive platter vibration dampener. In some embodiments, the cross-linked resilin compositions can be used as superior material for wheels, such as for skate boards, roller blades, or scooters. In some embodiments, the cross-linked resilin compositions provided herein can be used for safety and protective gear, such as padding for protective equipment such as helmets, elbow or knee pads, or hard hats, or as a protective outer layer to protect the skin from abrasions.

In some embodiments, the cross-linked resilin compositions provided herein can be used for automotive parts, e.g., suspension components such as bushings or shock absorbers, or for interior cushioning such as seat bolsters and lumbar support. In some embodiments, the cross-linked resilin compositions provided herein can be used for tires and inner tubes. In some embodiments, the cross-linked resilin compositions provided herein can be used for suberballs. In some embodiments, the cross-linked resilin compositions provided herein can be used for shoe insoles, midsoles, and outsoles. In some embodiments, the cross-linked resilin compositions provided herein can be used in a padded mat. In some embodiments, the cross-linked resilin compositions provided herein can be used for several types of gaskets or O-rings. In some embodiments, the cross-linked resilin compositions provided herein can be added to plastic items to increase their impact resistance. In some embodiments, the cross-linked resilin compositions provided herein can be used for protective cases, such as phone or tablet cases. In some embodiments, the cross-linked resilin compositions provided herein can be used for rubber stamps. In some embodiments, the cross-linked resilin compositions provided herein can be used for rollers. In some embodiments, the cross-linked resilin compositions provided herein can be used for rubber bands.

In some embodiments, the cross-linked resilin compositions provided herein can be used for shoe soles, basement flooring, noise protection for sound studios, car bumpers, cushion pads, door mats, yoga mats, drum pads, window wipers, car tires, fire hoses, electrical wiring insulation, rubber bands, rubber ducks, elastic gloves, cooking utensils, rain boots, teething toys, bicycle tires, watches, jars, gaskets, hair ties, flip-flops, phone cases, medicine balls, bouncy balls, seals for electronic devices to prevent contamination from water or dust, refrigerator or freezer door seals, seals to prevent air flow in or out of a chamber, trampolines, pacifiers, window seals, halloween masks, garden hoses, table tennis rackets, conveyer belts, ducting, stamps, balloons, cosmetic compositions for the care and protection of skin and hair, nail compositions, or preparations for decorative cosmetics.

Suitable skin cosmetic compositions are, for example, face tonics, facial masks, e.g., sheet masks, deodorants, and other cosmetic lotions. Compositions for use in decorative cosmetics include, for example, concealing sticks, stage make-up, mascara and eye shadows, lipsticks, kohl pencils, eyeliners, blushers, powders, and eyebrow pencils.

Depending on the field of use, the compositions described herein can be applied in a form suitable for skincare, such as, for example, as cream, foam, gel, stick, mousse, milk, spray (pump spray or propellant-containing spray) or lotion.

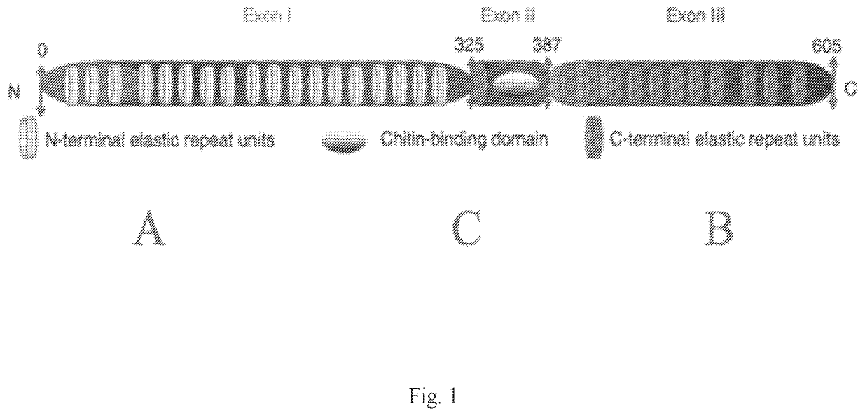

FIG. 1 illustrates an example of a native resilin, which contains an N-terminal A-domain comprising a plurality of repeat units comprising the consensus amino acid sequence YGXP (“A-repeat”), where X is any amino acid; a chitin-binding type RR-2 (C) domain (Pfam reference PF00379; Rebers J E & Willis, J H. A conserved domain in anthropod cuticular proteins binds chitin. Insect Biochem Mol Biol 31:1083-1093); and a C-terminal B-domain comprising a plurality of repeat units comprising the consensus amino acid sequence UYZXZ (“B-repeat”), where U is glycine or serine; Z is serine, glycine, arginine, or proline; and X is any amino acid. Not all naturally occurring resilins have A-, C-, and B-domains. Native resilins produced by various insects typically have inexact repeats (i.e., quasi-repeats) within the A- and/or B-domains with some amino acid variation between the quasi-repeats.

In some embodiments, the recombinant resilins provided herein comprise one or more A-repeats. In some embodiments, the recombinant resilins comprise N-terminal A-domains comprising a plurality of blocks of A-repeat and/or quasi-A-repeat amino acid sub-sequences each with the consensus sequence SXXYGXP (SEQ ID NO: 50), where S is serine, X is an amino acid, Y is tyrosine, G is glycine, and P is proline.

In some embodiments, the recombinant resilins provided herein comprise one or more B-repeats. In some embodiments, the recombinant resilins comprise a C-terminal B-domain comprising a plurality of blocks of B-repeat and/or quasi-B-repeat amino acid sub-sequences each with the consensus sequence GYZXZZX and/or SYZXZZX, where G is glycine; Y is tyrosine; Z is serine, glycine, proline, or arginine; S is serine; and X is any amino acid.

In some embodiments, the recombinant resilins provided herein comprise one or more A-repeats. In some such embodiments, the recombinant resilins comprise between 1 and 100 A-repeats, or from 2 to 50 A-repeats, or from 5 to 50 A-repeats, or from 5 to 20 A-repeats.

In some embodiments, the recombinant resilins comprise one or more consensus sequences described by the formula,

(1)
(SEQ ID NO: 64)
(X₁-X₂-X₃-X₄)ₙ

wherein the brackets delineate a repeat or quasi-repeat of the consensus sequence;
wherein n describes the number of A-repeats or quasi-A-repeats, and is from 1 to 100, or from 2 to 50, or from 5 to 50, or from 5 to 20;
wherein X₁ is a motif that is 4 amino acids in length, wherein the first amino acid of X₁ is Y, and wherein the remaining amino acids of X₁ are GAP, GLP, GPP, GTP, or GVP;
wherein X₂ is a motif that is from 3 to 20 amino acids in length;
wherein X₂ comprises GGG, GGGG (SEQ ID NO: 51), N, NG, NN, NGN, NGNG (SEQ ID NO: 52), GQGG (SEQ ID NO: 53), GQGN (SEQ ID NO: 54), GQGQ (SEQ ID NO: 55), GQGQG (SEQ ID NO: 56), or 3 or more glycine residues, or wherein 50% or more of the residues of X₂ are either glycine or asparagine, or wherein 60% or more of the residues of X₂ are either glycine or asparagine, or wherein 70% or more of the residues of X₂ are either glycine or asparagine, or wherein 80% or more of the residues of X₂ are either glycine or asparagine;
wherein X₃ is a motif that is from 2 to 4 amino acids in length, wherein X₃ is GG, LS, APS, GAG, GGG, KPS, RPS, or GGGG (SEQ ID NO: 51); and
wherein X₄ is a motif that is from 1 to 2 amino acids in length, wherein X₄ is S, D, T, N, L, DS, DT, LS, SS, ST, TN, or TS.

In some such embodiments, the recombinant resilins comprise motifs X₁, X₂, X₃, and X₄ whereas in other embodiments, the recombinant resilins comprise motifs X₁, X₂, X₃, or X₄, or combinations thereof.

In some embodiments, the recombinant resilins provided herein comprise one or more B-repeats. In some such embodiments, the recombinant resilins comprise between 1 and 100 B-repeats, or from 2 to 50 A-repeats, or from 5 to 50 A-repeats, or from 5 to 20 A-repeats.

In some embodiments, the recombinant resilins comprise one or more consensus sequences described by the formula,

(2)
(SEQ ID NO: 65)
(X₁₁-X₁₂-X₁₃)ₘ

wherein the brackets delineate a repeat or quasi-repeat of the consensus sequence;
wherein m describes the number of B-repeats or quasi-B-repeats, and is from 1 to 100;
wherein X₁₁ is a motif that is from 1 to 5 amino acids in length, the first amino acid is Y, and where the remaining amino acids can comprise GAP, GPP, SSG, or SGG;
wherein X₁₂ is a motif that is from 2 to 5 amino acids in length and comprises GQ, GN, RPG, RPGGQ (SEQ ID NO: 57), RPGGN (SEQ ID NO: 58), SSS, SKG, or SN; and
wherein X₁₃ is a motif that is from 4 to 30 amino acids in length and comprises GG, DLG, GFG, GGG, RDG, SGG, SSS, GGSF (SEQ ID NO: 59), GNGG (SEQ ID NO: 60), GGAGG (SEQ ID NO: 61), or 3 or more glycine residues, or 30% or more of the residues are glycine, or 40% or more of the residues are glycine, or 50% or more of the residues are glycine, or 60% or more of the residues are glycine.

In some such embodiments, the recombinant resilins comprise motifs X₁₁, X₁₂, and X₁₃ whereas in other such embodiments, the recombinant resilins comprise motifs X₁₁, X₁₂, or X₁₃, or combinations thereof.

In some embodiments, the recombinant resilins provided herein comprise one or more A-repeats, one or more B-repeats, and/or one or more C-domain. In some embodiments, the recombinant resilins comprise one or more A-repeats or one or more B-repeats but not both. In some embodiments, the recombinant resilins comprise one or more A-repeats but not B-repeats or C-domains. In some embodiments, the recombinant resilins comprise one or more B-repeats but not A-repeats or C-domains. In embodiments in which the recombinant resilins comprise a C-domain, the C-domain can be situated either on the N-terminal or the C-terminal sides of the A-repeats or B-repeats, or between the A-repeats and the B-repeats.

In some embodiments, the recombinant resilins further comprise the sequence XXEPPVSYLPPS (SEQ ID NO: 62), where X is any amino acid. In some such embodiments, the sequence is located on the N-terminal side of an A-repeat or B-repeat.

In some embodiments, the recombinant resilins are full-length native resilins expressed in a non-native environment. In some embodiments, the recombinant resilins comprise a truncated version of native resilins. In some embodiments, the truncated native resilins comprise at least one A-repeat. In some embodiments, the truncated native resilins comprise at least one B-repeat. Non-limiting examples of full-length and truncated native resilins are provided as SEQ ID NOs: 1 through 44. In some embodiments, the recombinant resilins are full-length Drosophila sechellia resilin (SEQ ID NO: 1). In some embodiments, the recombinant resilins are truncated Acromyrmex echinatior resilin (SEQ ID NO: 4). In some embodiments, the recombinant resilins are full-length or truncated native resilins that are cross-linked in a non-native manner (e.g., less or more cross-linking, cross-linking via different amino acid residues).

In some embodiments, the recombinant resilins are modified full-length or truncated native resilins. In some embodiments, the recombinant resilins are at least 60%, at least 65%, at least 70%, at least 75%, at least 80%, at least 85%, at least 90%, at least 95%, or at least 98% identical to a full-length or truncated native resilin. In some embodiments, the recombinant resilins are at least 60%, at least 65%, at least 70%, at least 75%, at least 80%, at least 85%, at least 90%, at least 95%, or at least 98% identical to full-length Drosophila sechellia resilin (SEQ ID NO: 1). In some embodiments, the recombinant resilins are at least 60%, at least 65%, at least 70%, at least 75%, at least 80%, at least 85%, at least 90%, at least 95%, or at least 98% identical to truncated Acromyrmex echinatior resilin (SEQ ID NO: 4).

There are a number of different algorithms known in the art which can be used to measure nucleotide sequence or protein sequence identity. For instance, polynucleotide sequences can be compared using FASTA, Gap, or Bestfit, which are programs in Wisconsin Package Version 10.0, Genetics Computer Group (GCG), Madison, Wis. FASTA provides alignments and percent sequence identity of the regions of the best overlap between the query and search sequences. See, e.g., Pearson, Methods Enzymol. 183:63-98, 1990 (hereby incorporated by reference in its entirety). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410, 1990; Gish and States, Nature Genet. 3:266-272, 1993; Madden et al., Meth. Enzymol. 266:131-141, 1996; Altschul et al., Nucleic Acids Res. 25:3389-3402, 1997; Zhang and Madden, Genome Res. 7:649-656, 1997, especially blastp or tblastn (Altschul et al., Nucleic Acids Res. 25:3389-3402, 1997.

In some embodiments, the modified resilins differ from full-length or truncated native resilins in amino acid residues that are post-translationally modified (e.g., glycosylated, phosphorylated) such that the modified resilins have one or more different locations and/or different amounts and/or different types of post-translational modifications than the full-length or truncated native resilins. In some embodiments, the modified resilins differ from full-length or truncated native resilins in amino acid residues that are involved in cross-linking such that the modified resilins have one or more different locations and/or different amounts and/or different types of amino acids that are involved in cross-linking than full-length or truncated native resilins. In some such embodiments, the modified resilins differ from the full-length or truncated native resilin in comprising one or more additional or fewer tyrosine residues, one or more additional or fewer lysine residues, and/or one or more additional or fewer cysteine residues.

In some embodiments, the recombinant resilins comprise concatenated native or truncated native resilins or concatenated modified resilins. In some embodiments, the concatenated native or truncated native resilins or concatenated modified resilins comprise at least 2 A-repeats (e.g., 2, 3, 4, 5, 6, 7, 8, 9, 10, or more). In some embodiments, the concatenated truncated native resilins or concatenated modified resilins comprise at least 2 B-repeats (e.g., 2, 3, 4, 5, 6, 7, 8, 9, 10, or more).

### Protein Quantification and Purity Analysis

In some embodiments, the total amount of full length resilin in a composition is determined by size exclusion chromatography (SEC), Reverse Phase High Performance Liquid Chromatography or other methods as known in the art (e.g. quantitative Western blots). In various embodiments, the same or similar assays may be used to assess the relative amount of full length resilin in its monomeric and aggregate forms.

As described herein, in a specific embodiment, data characterizing the relative amount of the predominant species of resilin (i.e. monomeric full length resilin) in a resilin composition is determined from Size Exclusion Chromatography (SEC) as follows: resilin powder is dissolved in 5M Guanidine Thiocyanate and injected onto a Yarra SEC-3000 SEC-HPLC column to separate constituents on the basis of molecular weight. Refractive index is used as the detection modality. BSA is used as a general protein standard with the assumption that >90% of all proteins demonstrate dn/dc values (the response factor of refractive index) within ˜7% of each other. Poly(ethylene oxide) is used as a retention time standard, and a BSA calibrator is used as a check standard to ensure consistent performance of the method. A range corresponding to monomeric full length resilin is assessed from 60-40 kDa; higher peaks observed which may correspond to aggregate or polymerized full length resilin are not included in this quantification. The relative percentage of this resilin peak is reported as mass % and area % to determine the amount of full length resilin in a composition as a portion of total resilin.

### Cross-Linking

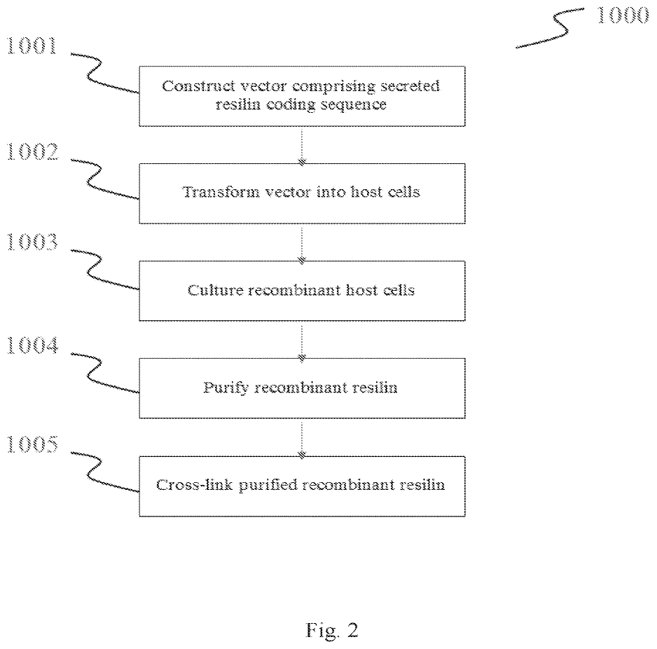

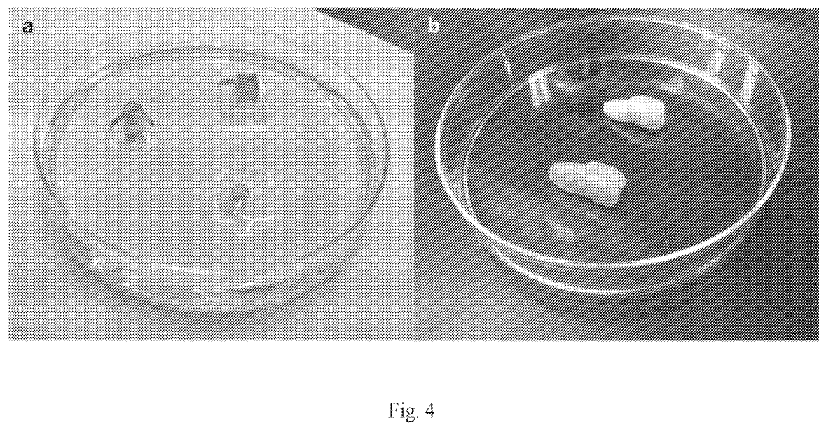

In some embodiments, the methods provided herein further comprise the step of cross-linking the recombinant resilins to obtain the recombinant resilin compositions provided herein (step 1005 in FIG. 2). The recombinant resilin in the desired solvent with cross-linking agents can be filled into small shaped molds to control the shape of the resulting solid after cross-linking. Examples of resulting recombinant resilin solids are shown in FIG. 4.

In some embodiments, cross-linking is achieved via tyrosine residues. Resilin has a tyrosine residue every 15-24 amino acids throughout most of its length. In some embodiments, cross-linking of resilin creates di- and tri-tyrosine crosslinking in resilin to form a resilin solid.

In other embodiments, cross linking is achieved via lysine residues. In some embodiments, cross linking is achieved via cysteine residues. In some embodiments, cross-linking employs transglutaminase (see, for example, Kim Y, Gill E E, Liu J C. Enzymatic Cross-Linking of Resilin-Based Proteins for Vascular Tissue Engineering Applications. Biomacromolecules. 17(8):2530-9). In some embodiments, cross-linking employs poly(ethylene glycol) (PEG) (McGann C L, Levenson E A, Kiick K L. Macromol. Chem. Phys. 2013, 214, 203-13; McGann C L, Akins R E, Kiick K L. Resilin-PEG Hybrid Hydrogels Yield Degradable Elastomeric Scaffolds with Heterogeneous Microstructure. Biomacromolecules. 2016; 17(1):128-40).

In some embodiments, recombinant resilin is cross-linked via enzymatic cross-linking (e.g., using horseradish peroxidase). While this method can efficiently cross-link large solutions of resilin, the resulting cross-linked product comprises covalently incorporated active enzyme in the cross-linked resilin solid. This yields radical chain reactions that degrade the protein backbone of the resilin.

In other embodiments, recombinant resilin is cross-linked via photochemical cross-linking (see, for example, Elvin C M, Carr A G, Huson M G, Maxwell J M, Pearson R D, Vuocolo T, Liyou N E, Wong D C C, Merritt D J, Dixon N E. Nature 2005, 437, 999-1002; Whittaker J L, Dutta N K, Elvin C M, Choudhury N R. Journal of Materials Chemistry B 2015, 3, 6576-79; Degtyar E, Mlynarczyk B, Fratzl P, Harrington M J. Polymer 2015, 69, 255-63).

This cross-linking reaction also results in the incorporation of an active catalyst into the resilin solid that is difficult to completely dialyze out of the material, uses relatively high catalyst loading, and is not efficient for reactions where photoactivation throughout the mold may be difficult.

Provided herein are new recombinant resilin cross-linking chemistries that prevent degradation and make solid substances with some mechanical properties preferred for certain applications where the amount and form of energy absorption is important.

In preferred embodiments, recombinant resilin is cross-linked via a solvent comprising ammonium persulfate and application of heat (e.g., incubation at a temperature of about 80° C. for about 2.5 hours). This cross-linking reaction is efficient and does not leave an active catalyst behind in the resilin solid, resulting in a composition with less degraded protein.

In some embodiments, the final concentration of ammonium persulfate for the resilin cross-linking reaction is about 30 to 40 mM. In some embodiments, the cross-linking solution with ammonium persulfate and resilin is incubated (i.e., exposed to heat) for at least 15 minutes, at least 30 minutes, at least 60 minutes, at least 90 minutes, or at least 2 hours to facilitate sufficient cross-linking to form a solid with desirable mechanical properties. In some embodiments, other persulfates are used.

In some embodiments, the heat applied to the ammonium persulfate and resilin composition solution is at least 40° C., at least 45° C., at least 50° C., at least 55° C., at least 60° C., at least 65° C., at least 70° C., at least 75° C., or at least 80° C. In some embodiments, the heat applied to the cross-linking solution of ammonium persulfate and resilin is from 50° C.-90° C. or from 60° C.-90° C. or from 70° C.-90° C. or from 75° C. to 85° C. or from 70° C. to 80° C. In some embodiments, the cross-linking solution does not comprise an enzymatic catalyst. In some embodiments, the cross-linking solution does not comprise a photoactive catalyst.

In some embodiments, the cross-linking solution comprises at least 1%, 5%, 10%, 20%, 25%, 30%, 35%, 40%, 45%, or 50% resilin, from 1% to 100%, 90%, 80%, 70%, 60%, 50%, 40%, 30%, 20%, or 10%; from 10% to 100%, 90%, 80%, 70%, 60%, 50%, 40%, 30%, or 20%; from 20% to 100%, 90%, 80%, 70%, 60%, 50%, 40%, or 30%; from 30% to 100%, 90%, 80%, 70%, 60%, 50%, or 40%; from 40% to 100%, 90%, 80%, 70%, 60%, or 50%; from 50% to 100%, 90%, 80%, 70%, or 60%; from 60% to 100%, 90%, 80%, or 70%; from 70% to 100%, 90%, or 80%; from 80% to 100%, or 90%; or from 90% to 100% by weight of the total cross-linking solution. In preferred embodiments, resilin is dissolved in a cross-linking solution to a concentration of between 20% to 30% by weight resilin. In some embodiments the total resilin comprises at least 10%, at least 20%, at least 30%, at least 40%, at least 50%, at least 60%, at least 70%, at least 80%, or at least 90% full length resilin as measured by size exclusion chromatography (SEC).

### Solvent-Exchanged Resilin Solids

Cross-linked resilin can be formed in an aqueous solvent resulting in a composition that has a low hardness and elastic modulus that is less suitable for certain applications where energy absorption and stiffness are desired. In some embodiments, a solvent exchange is performed on cross-linked resilin compositions to replace an aqueous solvent with a polar nonaqueous solvent to provide desired material properties. In some embodiments, the polar nonaqueous solvent comprises glycerol, propylene glycol, ethylene glycol, or DMSO. In some embodiments, the nonaqueous solvents are water-miscible. In some embodiments the nonaqueous solvents are non-volatile solvents. In some embodiments, the nonaqueous solvents comprise molecules with a plurality of alcohol functional groups. In some embodiments the nonaqueous solvent comprises at least 50%, at least 60%, at least 70%, at least 80%, at least 90%, or 100% of a molecule or molecules with a molecular weight less than about 100 g/mol. As described herein, material properties of cross-linked resilin compositions, including elastic modulus, hardness, maximum elastic compressive load, resilience, and material lifetime/fatigue, can be tuned using solvent exchange. Solvents that are capable of doing solvent exchange with cross-linked resilin include solvents that dissolve resilin in its non-crosslinked form.

In some embodiments, a solvent exchange to replace an aqueous solvent of resilin with a polar nonaqueous solvent is performed in the presence of heat, e.g., at a temperature of about 60° C. In some embodiments the solvent exchange process is performed in a solution containing at least 1×, at least 2×, at least 5×, at least 10×, or at least 20× the volume of exchange solvent relative to the resilin solid. In some embodiments, the solvent exchange is performed for at least 1 hour, at least 2 hours, at least 4 hours, at least 8 hours, at least 16 hours, at least 24 hours, or at least 48 hours. In some embodiments, glycerol, propylene glycol, ethylene glycol, or DMSO are used as exchange solvents for cross-linked resilin solid compositions.

In some embodiments, the choice of exchange solvent and the concentration used is selected to achieve a desired tunable mechanical property, such as stiffness, from the solvent design. This can be selected depending on the desired application (e.g., shoe soles, golf balls, etc.).

In some embodiments, the cross-linked resilin composition formed by the above solvent exchange process and in a nonaqueous solvent is stable at room temperature for at least 10 days, at least 20 days, at least 30 days, at least 40 days, or at least 50 days. In some embodiments, the cross-linked resilin composition formed by the above solvent exchange process and in a nonaqueous solvent is resistant to mold growth for at least 10 days, at least 20 days, at least 30 days, at least 40 days, or at least 50 days. In some embodiments, the cross-linked resilin composition formed by the above solvent exchange process and in a nonaqueous solvent is resistant to dehydration for at least 7 days or at least 14 days. By contrast, water-based cross-linked resilin solids were observed to dehydrate completely within 2 weeks and to grow mold within 5-14 days when stored in an open environment under ambient conditions. In addition, a 10 mm by 5 mm disc of water-based resilin solid breaks when 220 lbs of force is applied whereas a 10 mm by 5 mm disc of a glycerol-based resilin solid does not break under 220 lbs of force.

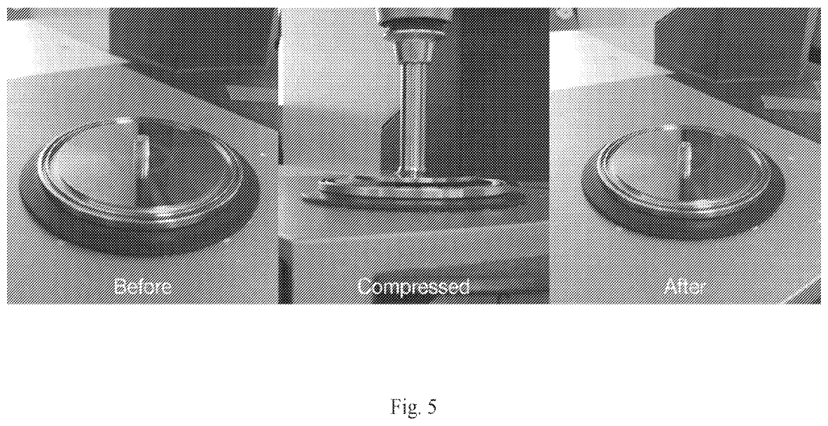

In some embodiments, relative material properties between consecutively tested materials can be tested using a rheometer. For example, a cross-linked resilin composition may be formed in a cylinder mold, and the resulting resilin cylinder is subjected to a compression test using a rheometer. A recombinant resilin cylinder could be compressed from an initial height of 7.3 mm (avg width 5.4 mm) to less than 0.66 mm without any breakage. As shown in FIG. 5, the cylinder returned to a height of 6.7 mm (avg width 5.6 mm) upon release of the compressive load.

In some embodiments, the polar nonaqueous solvent comprises at least 50%, at least 60%, at least 70%, at least 80%, at least 90%, at least 95%, at least 98%, or at least 99% of a polar nonaqueous composition by volume of said solvent. In some embodiments, the solvent comprises at least 50%, at least 60%, at least 70%, at least 80%, at least 90%, at least 95%, at least 98%, or at least 99% of glycerol, propylene glycol, ethylene glycol, or DMSO by volume of said solvent.

In some embodiments, the recombinant resilin composition provided herein has an elastic modulus greater than cross-linked recombinant resilin in an aqueous solvent.

In some embodiments, the cross-linked resilin compositions provided herein have a Shore OO Hardness of 50 or more, 40 or more, 30 or more, 20 or more, 10 or more, from 10 to 50, 40, 30, or 20; from 20 to 50, 40, or 30; from 30 to 50 or 40; or from 40 to 50; In some embodiments, hardness measurements in resilin are performed according to ASTM D2240. In some embodiments, the recombinant resilin composition comprises a hardness of at least 10 as measured using a Shore OO Durometer via ASTM D2240. In some embodiments, the recombinant resilin composition comprises a hardness of at least 30 as measured using a Shore OO Durometer via ASTM D2240. In some embodiments, the recombinant resilin composition comprises a hardness of from about 10 to about 50 as measured using a Shore OO Durometer via ASTM D2240. In some embodiments, the hardness of the cross-linked resilin composition is comparable to an orthopedic sole.

In some embodiments, the recombinant resilin composition comprises a rebound resilience from about 40% to about 60% as measured by ASTM D7121.

In some embodiments, the recombinant resilin composition comprises a compressive stress at 25% of about 6 psi to about 8 psi as measured by ASTM D575.

In some embodiments, the recombinant resilin composition does not undergo an elastic to plastic transition below 2 kN of compressive force as measured by a Zwick compression test.

In some embodiments, resilin solid material properties, such as resilience, compressive elastic modulus, tensile elastic modulus, shear modulus, extension to break, maximum tensile strength, hardness, stiffness, and rebound can be tuned based on the solvent used and how the solvent exchange is performed. For example, glycerol can be exchanged into the resilin solid either with a gradient or without, result in different resulting material properties. The concentration of resilin in the resilin solid and the amount of full length resilin as a portion of total resilin can also be adjusted to affect the material properties of the cross-linked resilin solid composition.

In some embodiments, solvent exchange to replace the water-based resilin composition (i.e, a cross-linked resilin composition in an aqueous solvent) with a polar nonaqueous-based resilin composition (i.e., a cross-linked resilin composition in a polar nonaqueous solvent) as described herein resulting in a stiffer material with a similar resilience and a similar elasticity.

In some embodiments, the compositions provided herein comprise at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%; between 10% and 100%, 90%, 80%, 70%, 60%, 50%, 40%, 30%, or 20%; between 20% and 100%, 90%, 80%, 70%, 60%, 50%, 40%, or 30%; between 30% and 100%, 90%, 80%, 70%, 60%, 50%, or 40%; between 40% and 100%, 90%, 80%, 70%, 60%, or 50%; between 50% and 100%, 90%, 80%, 70%, or 60%; between 60% and 100%, 90%, 80%, or 70%; between 70% and 100%, 90%, or 80%; between 80% and 100%, or 90%; or between 90% and 100% by weight of recombinant resilins. The recombinant resilins can be identical recombinant resilins or mixtures of recombinant resilins having at least 2 different amino acid sequences.

In some embodiments, the compositions provided herein comprise at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%; between 10% and 100%, 90%, 80%, 70%, 60%, 50%, 40%, 30%, or 20%; between 20% and 100%, 90%, 80%, 70%, 60%, 50%, 40%, or 30%; between 30% and 100%, 90%, 80%, 70%, 60%, 50%, or 40%; between 40% and 100%, 90%, 80%, 70%, 60%, or 50%; between 50% and 100%, 90%, 80%, 70%, or 60%; between 60% and 100%, 90%, 80%, or 70%; between 70% and 100%, 90%, or 80%; between 80% and 100%, or 90%; or between 90% and 100% by weight of full length recombinant resilins as a portion of total resilin in each composition.

In some embodiments, a recombinant resilin composition is a foam material. In some embodiments, a cross-linked resilin composition is a foam material, e.g., a foamed solid. In some embodiments, the foam material comprises at least 50%, at least 60%, at least 70%, at least 80%, at least 90%, at least 95%, at least 98%, or at least 99% porosity. As described herein, porosity refers to the percentage of the volume of interstices of a material to the volume of its mass. In some embodiments, density of the foam material as described herein is at least ⅓rd the density of a non-foam material, e.g., at least ½, at least ¾th, or at least twice the density of a non-foam cross-linked resilin composition. In some embodiments, average diameter of the bubbles in the foam material as described herein is about 0.01 mm to about 3 mm, e.g., about 0.02 mm to about 1 mm, about 0.05 mm to about 2 mm, about 0.1 mm to about 3 mm, about 0.2 mm to about 4 mm, about 0.5 mm to about 5 mm, about 1 mm to about 1.5 mm, or about 1.5 mm to about 2 mm.

In some embodiments, a recombinant resilin composition comprises one or more layers of foam material. In some embodiments, the one or more layers of foam material are configured in a desired order, e.g. a sequential or alternate configuration, wherein a supporting layer is positioned in between two layers of foam material. The one or more layers of foam material may be positioned in any manner known in the art, e.g., spray drying, injection molding. In some embodiments, the one or more layers of foam material includes one or more void spaces. The topography of each layer and the propensity of the one or more layers of foam material to nest together may differ. In some embodiments, the void spaces may provide flow channels on one or more sides to help guide air flow and provide additional absorbent capacity.

### Mechanical Properties

In other embodiments, the compositions provided herein have different properties compared to compositions comprising cross-linked resilins. In some embodiments, the compositions provided herein have similar properties compared to synthetic elastic materials. Non-limited examples of such properties include resilience, compressive elastic modulus, tensile elastic modulus, shear modulus, hardness, rebound, and compression set. Parameters that can be modified to obtain compositions with specific mechanical properties include, for example, the length and/or sequence of the recombinant resilins, the extent and/or type of post-translational modifications of the recombinant resilins, the extent and/or type of cross-linking of the recombinant resilins and the nature of the solvent of the cross-linked resilin composition.

In some embodiments, mechanical properties such as maximum tensile strength, compressive elastic modulus, tensile elastic modulus, shear modulus, extension to break and resilience can be measured using many different types of tensile and compression systems that conduct stress-strain measurements on elastomeric samples. The resulting stress-strain curves, including curves with hysteresis, can be measured in tension or compression. In some embodiments, tension and compression test systems can apply a strain to a sample and measure the resulting force using a load cell. In some embodiments, the mechanical properties can be measured at the macroscopic scale (e.g., using macroscopic compression testers), microscopic, or nanoscopic scale (e.g., using atomic-force microscopy (AFM) or nanoindentation measurements). In some embodiments, the compressive mechanical properties of elastomers can be measured according to the standard ASTM D575-91(2012) Standard Test Methods for Rubber Properties in Compression. Mechanical measurements of elastomers in tension can be performed using ASTM D412-15a Standard Test Methods for Vulcanized Rubber and Thermoplastic Elastomers—Tension. In some embodiments, tear strength of elastomers can be performed using ASTM D624-00 Standard Test Method for Tear Strength of Conventional Vulcanized Rubber and Thermoplastic Elastomers. In some embodiments, mechanical properties of slab, bonded, and molded elastomers can be performed using ASTM D3574-11 Standard Test Methods for Flexible Cellular Materials-Slab, Bonded, and Molded Urethane Foams. In some embodiments, the mechanical properties of elastomers can be measured using ASTM D5992-96(2011) Standard Guide for Dynamic Testing of Vulcanized Rubber and Rubber-Like Materials Using Vibratory Methods.

In some embodiments, the compositions provided herein have a resilience of greater than 50%, greater than 60%, greater than 70%, greater than 80%, greater than 90%, or greater than 95%; from 50% to 100%, 90%, 80%, 70%, or 60%; from 60% to 100%, 90%, 80%, or 70%; from 70% to 100%, 90%, or 80%; from 80% to 100%, or 90%; from 90% to 100%; from 95% to 100%, from 90% to 99%, or from 95% to 99%.

In some embodiments, the compositions provided herein have a compressive elastic modulus of less than 10 MPa, less than 7 MPa, less than 5 MPa, less than 2 MPa, less than 1 MPa, less than 0.5 MPa, or less than 0.1 MPa; from 0.01 MPa to 10 MPa, 7 MPa, 5 MPa, 2 MPa, 1 MPa, 0.5 MPa, or 0.1 MPa; from 0.1 MPa to 10 MPa, 7 MPa, 5 MPa, 2 MPa, 1 MPa, or 0.5 MPa; from 0.5 MPa to 10 MPa, 7 MPa, 5 MPa, 2 MPa, or 1 MPa; from 1 MPa to 10 MPa, 7 MPa, 5 MPa, or 2 MPa; from 2 MPa to 10 MPa, 7 MPa, or 5 MPa; from 5 MPa to 10 MPa, or 7 MPa; or from 7 MPa to 10 MPa. In some embodiments, the compressive elastic modulus of a composition can be measured as defined by the ASTM D575-91(2012) Standard Test Methods for Rubber Properties in Compression.

In some embodiments, the compositions provided herein have a tensile elastic modulus of less than 10 MPa, less than 7 MPa, less than 5 MPa, less than 2 MPa, less than 1 MPa, less than 0.5 MPa, or less than 0.1 MPa; from 0.01 MPa to 10 MPa, 7 MPa, 5 MPa, 2 MPa, 1 MPa, or 0.5 MPa; from 0.5 MPa to 10 MPa, 7 MPa, 5 MPa, 2 MPa, or 1 MPa; from 1 MPa to 10 MPa, 7 MPa, 5 MPa, or 2 MPa; from 2 MPa to 10 MPa, 7 MPa, or 5 MPa; from 5 MPa to 10 MPa, or 7 MPa; or from 7 MPa to 10 MPa.

In some embodiments, the compositions provided herein have a shear modulus of less than 1 MPa, less than 100 kPa, less than 50 kPa, less than 20 kPa, less than 10 kPa, or less than 1 kPa; from 0.1 kPa to 1 MPa, 100 kPa, 50 kPa, 20 kPa, 10 kPa, or 1 kPa; from 1 kPa to 1 MPa, 100 kPa, 50 kPa, 20 kPa, or 10 kPa; from 10 kPa to 1 MPa, 100 kPa, 50 kPa, or 20 kPa; from 20 kPa to 1 MPa, 100 kPa, or 50 kPa; from 50 kPa to 1 MPa, or 100 kPa; or from 100 kPa to 1 MPa.

In some embodiments, the compositions provided herein have an extension to break of greater than 1%, greater than 10%, greater than 50%, greater than 100%, greater than 300%, or greater than 500%; from 1% to 500%, 300%, 100%, 50%, or 10%; from 10% to 500%, 300%, 100%, or 50%; from 50% to 500%, 300%, or 100%; from 100% to 500%, or 300%; or from 300% to 500%.

In some embodiments, the compositions provided herein have a maximum tensile strength of greater than 0.1 kPa, greater than 1 kPa, greater than 2 kPa, greater than 5 kPa, or greater than 10 kPa; from 0.1 kPa to 100 kPa, 10 kPa, 5 kPa, 2 kPa, or 1 kPa; from 1 kPa to 100 kPa, 10 kPa, 5 kPa, or 2 kPa; from 2 kPa to 100 kPa, 10 kPa, or 5 kPa; from 5 kPa to 100 kPa, or 10 kPa; or from 10 kPa to 100 kPa.

In some embodiments, mechanical properties such as hardness and compressive elastic modulus can be measured using indentation and nanoindentation measurement systems. In some embodiments, indentation measurements utilizing a tip to indent the sample to a given amount of strain are used to measure the hardness and compressive elastic modulus of resilin, and the resulting force is measured using a load cell. In some embodiments, different tip shapes can be used including Vickers and Berkovich shaped tips. In some embodiments, the hardness measured by indentation techniques is characterized by the relation, Hardness=(Peak Force)/(Contact Area).

In some embodiments, the hardness in polymers, elastomers, and rubbers can be measured using a durometer. In some embodiments the hardness of an elastomer can be measured using the standard ASTM D2240, which recognizes twelve different durometer scales using combinations of specific spring forces and indentor configurations. The most common scales are the Shore OO, A and D Hardness Scales. Hardness scales range from 0 to 100, where 0 is softer material and 100 is harder material.

In some embodiments, the compositions provided herein have a Shore OO Hardness of less than 90, less than 80, less than 70, less than 60, less than 50, less than 40, less than 30, or less than 20; from 10 to 90, 80, 70, 60, 50, 40, 30, or 20; from 20 to 90, 80, 70, 60, 50, 40, or 30; from 30 to 90, 80, 70, 60, 50, or 40; from 40 to 90, 80, 70, 60, or 50; from 50 to 90, 80, 70, or 60; from 60 to 90, 80, or 70; from 70 to 90, or 80; or from 80 to 90. In some embodiments, hardness measurements in resilin are performed according to ASTM D2240.

As used here, the term “rebound” refers to a particular measure of resilience. In some embodiments, rebound can be measured with a number of different tools including pendulum tools and dropped balls. In the pendulum type measurements, RB, commonly called percentage rebound, is determined from the equation:

$$RB = \frac{[ 1 - cos ( angle of rebound ) ]}{[ 1 - cos ( originial angle ) ]} \times 100$$

The rebound resilience can be calculated as:

$$R = \frac{h}{H}$$

where h=apex height of the rebound, and H=initial height. The rebound resilience can also be determined by the measurement of the angle of rebound. Some examples of test methods for determining rebound in elastomers are ASTM D2632-15 and ASTM D7121-05(2012).

In some embodiments, the compositions provided herein have a rebound greater than 50%, greater than 60%, greater than 70%, greater than 80%, greater than 90%, or greater than 95%; from 50% to 100%, 90%, 80%, 70%, or 60%; from 60% to 100%, 90%, 80%, or 70%; from 70% to 100%, 90%, or 80%; from 80% to 100%, or 90%; from 90% to 100%; from 95% to 100%, from 90% to 99%, or from 95% to 99%. In some embodiments, rebound measurements in resilin are performed according to ASTM D2632-15, or ASTM D7121-05(2012).

As used herein, the term “compression set” refers to a measure of the permanent deformation remaining after an applied force is removed. In some embodiments, compression set can be measured in different ways, including compression set under constant force in air (referred to as Compression Set A), compression set under constant deflection in air (referred to as Compression Set B), and compression set under constant deflection in air considering material hardness (referred to as Compression Set C). Compression Set A (C_{A}) is calculated by the following expression: C_{A}=[(tₒ−t₁)/tₒ]×100, where to is the original specimen thickness, and tᵢ is the specimen thickness after testing). Compression set B (C_{B}) is given by C_{B}=[(tₒ−tᵢ)/(tₒ−tₙ)]×100, where to is the original specimen thickness, tᵢ is the specimen thickness after testing, and tₙ is the spacer thickness or the specimen thickness during the test. Some examples of test methods for determining compression set in elastomers are ASTM D3574-11 and ASTM D395
16.

In some embodiments, the compositions provided herein have a Compression Set A or a Compression Set B of greater than 50%, greater than 60%, greater than 70%, greater than 80%, greater than 90%, or greater than 95%; from 50% to 100%, 90%, 80%, 70%, or 60%; from 60% to 100%, 90%, 80%, or 70%; from 70% to 100%, 90%, or 80%; from 80% to 100%, or 90%; from 90% to 100%; from 95% to 100%, from 90% to 99%, or from 95% to 99%. In some embodiments, compression set measurements in resilin are performed according to ASTM D3574-11 and ASTM D395-16.

The processing and forming of resilin into products can take many forms for different applications. Accordingly, the compositions provided herein can have any shape and form, including but not limited to gels, porous sponges, films, machinable solids, cast forms, molded forms, and composites.

As used herein, the term “density” refers to the mass of the sample divided by the volume. In some embodiments, the density of an elastomer can be determined using a pycnometer with alcohol in place of water to eliminate air bubbles. In some embodiments, the density of an elastomer can be determined using a hydrostatic method. As used herein, the term “compressed volume density” refers to the ratio of the sample mass to the compressed volume of the sample, where the “compressed volume” is defined as the final equilibrium volume attained by an elastomeric sample when it is subjected to a compressive force sufficient to cause it to flow until it fully conforms to the surrounding shape of the piston-cylinder test chamber enclosure. In some embodiments, the compressed volume density of an elastomer can be determined using a compressed volume densimeter.

In some embodiments, the compositions provided herein have a density or a compressed volume density are from 0.5 mg/cm³ to 2.0 mg/cm³, or from 1.0 mg/cm³ to 1.5 mg/cm³, or from 1.1 mg/cm³ to 1.4 mg/cm³, or from 1.2 mg/cm³ to 1.35 mg/cm³. In some embodiments, he determination of the density or the compressed volume density of elastomers can be performed using ASTM D297-15 Standard Test Methods for Rubber Products-Chemical Analysis.

The compositions provided herein have a number of uses, including but not limited to applications in aerospace, automotive, sporting equipment, vibration isolation, footwear, and clothing among others. Some applications from these categories are listed as non-limiting examples. Due to the desirable elastic efficiency, resilin can be used as an energy storage device (e.g., a rubber band) for storing and recovering mechanical energy. Automobile suspension systems can be improved by application of resilin bushings to keep more tire contact on the road when going over bumps and through potholes at speed. Additionally, there are a number of sporting equipment applications for resilin with differently tuned mechanical properties including cores of golf balls, tennis racket grips, golf club grips, and table tennis paddles.

An application of particular interest is footwear due to the unique properties of resilin compositions provided herein. As an insole or midsole, resilin can improve the comfort and bioefficiency of shoes by cushioning the foot strike and restoring more of the energy from that footstrike as forward momentum. As a midsole, resilin can make up the entire midsole or be encapsulated within another material to complement its properties (e.g., an abrasion or wear resistant material, or a material tuned for traction). The resilin midsole can also contain a plurality of resilin materials with differently tuned mechanical properties that work in concert to provide enhanced performance (e.g., softer heel strike area and firmer arch support).

### Vectors, Host Cells, and Fermentations

Further provided herein are vectors encoding recombinant resilins, recombinant host cells comprising such vectors, and fermentations comprising such recombinant host cells and recombinant resilins.

In some embodiments, the vectors provided herein comprise secreted resilin coding sequences, which encode a resilin polypeptide fused at its N-terminus to a secretion signal and optionally at its C-terminus to a tag peptide or polypeptide. In some embodiments, the vectors comprise secreted resilin coding sequences that are codon-optimized for expression in a particular host cell.

Suitable secretion signals are secretion signals that mediate secretion of polypeptides in the recombinant host cells provided herein. Non-limiting examples of suitable secretion signals are the secretion signals of the alpha mating factor (α-MF) of Saccharomyces cerevisiae, acid phosphatase (PHO1) of Pichia pastoris, and phytohemagglutinin (PHA-E) from the common bean Phaseolus vulgaris. Additional secretion signals are known in the art, or can be identified by identification of proteins secreted by a host cell followed by genomic analysis of the secreted proteins and identification of the non-translated N-terminal sequences (see, for example, Huang et al. A proteomic analysis of the Pichia pastoris secretome in methanol-induced cultures. Appl Microbiol Biotechnol. 2011 April; 90(1):235-47).

The resilins encoded by the secreted resilin coding sequences can be further fused to tag peptides or polypeptides. Non-limiting examples of tag peptides or polypeptides include affinity tags (i.e., peptides or polypeptides that bind to certain agents or matrices), solubilization tags (i.e., peptides or polypeptides that assist in proper folding of proteins and prevent precipitation), chromatography tags (i.e., peptides or polypeptides that alter the chromatographic properties of a protein to afford different resolution across a particular separation techniques), epitope tags (i.e., peptides or polypeptides that are bound by antibodies), fluorescence tags (i.e., peptides or polypeptides that upon excitation with short-wavelength light emit high-wavelength light), chromogenic tags (i.e., peptides or polypeptides that absorb specific segments of the visible light spectrum), enzyme substrate tags (i.e., peptides or polypeptides that are the substrates for specific enzymatic reactions), chemical substrate tags (i.e., peptides or polypeptides that are the substrates for specific chemical modifications), or combinations thereof. Non-limiting examples of suitable affinity tags include maltose binding protein (MBP), glutathione-S-transferase (GST), poly(His) tag, SBP-tag, Strep-tag, and calmodulin-tag. Non-limiting examples of suitable solubility tags include thioredoxin (TRX), poly (NANP), MBP, and GST. Non-limiting examples of chromatography tags include polyanionic amino acids (e.g., FLAG-tag [GDYKDDDDKDYKDDDDKDYKDDDDK (SEQ ID NO: 45)]) and polyglutamate tag. Non-limiting examples of epitope tags include V5-tag, VSV-tag, Myc-tag, HA-tag, E-tag, NE-tag, and FLAG-tag. Non-limiting examples of fluorescence tags include green fluorescent protein (GFP), blue_fluorescent protein (BFP), cyan fluorescent protein (CFP), yellow fluorescent protein (YFP), orange fluorescent protein (OFP), red fluorescent protein (RFP), and derivatives thereof. Non-limiting examples of chromogenic tags include non-fluorescent members of the GFP-like family of proteins (e.g., BlitzenBlue, DonnerMagenta; DNA2.0, Neward, CA). Non-limiting examples of enzyme substrate tags include peptides or polypeptides comprising a lysine within a sequence suitable for biotinilation (e.g., AviTag, Biotin Carboxyl Carrier Protein [BCCP]). Non-limiting examples of chemical substrate tags include substrates suitable for reaction with FIASH-EDT2. The fusion of the C-terminal peptide or polypeptide to the resilin can be cleavable (e.g., by TEV protease, thrombin, factor Xa, or enteropeptidase) on non-cleavable.

In some embodiments, the vectors comprise single secreted resilin coding sequences. In other embodiments, the vectors comprise 2 or more (e.g., 3, 4, or 5) secreted resilin coding sequences. In some such embodiments, the secreted resilin coding sequences are identical. In other such embodiments, at least 2 of the secreted resilin coding sequences are not identical. In embodiments in which at least 2 of the secreted resilin coding sequences are not identical, the at least 2 secreted resilin coding sequences can differ from each other in the resilins and/or in the secretion signals and/or the optional tag peptides or polypeptides they encode.

In some embodiments, the vectors comprise promoters that are operably linked to the secreted resilin coding sequences such that they drive the expression of the secreted resilin coding sequences. The promotors can be constitutive promoters or inducible promoters. In some embodiments, induction of the inducible promoter occurs via glucose repression, galactose induction, sucrose induction, phosphate repression, thiamine repression, or methanol induction. Suitable promoters include promoters that mediate expression of proteins in the recombinant host cells provided herein. Non-limiting examples of suitable promoters include the AOX1 promoter, GAP promoter, LAC4-PBI promoter, T7 promoter, TAC promoter, GCW14 promoter, GAL1 promoter, λPL promoter, λPR promoter, beta-lactamase promoter, spa promoter, CYC1 promoter, TDH3 promoter, GPD promoter, TEF1 promoter, ENO2 promoter, PGL1 promoter, SUC2 promoter, ADH1 promoter, ADH2 promoter, HXT7 promoter, PHO5 promoter, and CLB1 promoter. Additional promoters that can be used to facilitate expression of the secreted resilin coding sequences are known in the art.

In some embodiments, the vectors comprise terminators that are operably linked to the secreted resilin coding sequences such that they effect termination of transcription of the secreted resilin coding sequences. Suitable terminators include terminators that terminate transcription in the recombinant host cells provided herein. Non-limiting examples of suitable terminators include the AOX1 terminator, PGK 1 terminator, and TPS1 terminator. Additional terminators that effect termination of transcription of the secreted resilin coding sequences are known in the art.

In embodiments in which the vectors comprise 2 or more resilin coding sequences, the 2 or more resilin coding sequences can be operably linked to the same promoters and/or terminators or to 2 or more different promoters and/or terminators.

The vectors provided herein can further comprise elements suitable for propagation of the vectors in recombinant host cells. Non-limiting examples of such elements include bacterial origins of replication and selection markers (e.g., antibiotic resistance genes, auxotrophic markers). Bacterial origins of replication and selection markers are known in the art. In some embodiments, the selection marker is a drug resistant marker. A drug resistant maker enables cells to detoxify an exogenously added drug that would otherwise kill the cell. Illustrative examples of drug resistant markers include but are not limited to those for resistance to antibiotics such as ampicillin, tetracycline, kanamycin, bleomycin, streptomycin, hygromycin, neomycin, Zeocin™, and the like. In some embodiments, the selection marker is an auxotrophic marker. An auxotrophic marker allows cells to synthesize an essential component (usually an amino acid) while grown in media that lacks that essential component. Selectable auxotrophic gene sequences include, for example, hisD, which allows growth in histidine-free media in the presence of histidinol. Other selection markers suitable for the vectors of the present invention include a bleomycin-resistance gene, a metallothionein gene, a hygromycin B-phosphotransferase gene, the AURI gene, an adenosine deaminase gene, an aminoglycoside phosphotransferase gene, a dihydrofolate reductase gene, a thymidine kinase gene, and a xanthine-guanine phosphoribosyltransferase gene.

The vectors of the present invention can further comprise targeting sequences that direct integration of the secreted resilin coding sequences to specific locations in the genome of host cells. Non-limiting examples of such targeting sequences include nucleotide sequences that are identical to nucleotide sequences present in the genome of a host cell. In some embodiments, the targeting sequences are identical to repetitive elements in the genome of host cells. In some embodiments, the targeting sequences are identical to transposable elements in the genome of host cells.

In some embodiments, recombinant host cells are provided herein that comprise the vectors described herein. In some embodiments, the vectors are stably integrated within the genome (e.g., a chromosome) of the recombinant host cells, e.g., via homologous recombination or targeted integration. Non-limiting examples of suitable sites for genomic integration include the Tyl loci in the Saccharomyces cerevisiae genome, the rDNA and HSP82 loci in the Pichia pastoris genome, and transposable elements that have copies scattered throughout the genome of the recombinant host cells. In other embodiments, the vectors are not stably integrated within the genome of the recombinant host cells but rather are extrachromosomal.

Recombinant host cells can be of mammalian, plant, algae, fungi, or microbe origin. Non-limiting examples of suitable fungi include methylotrophic yeast, filamentous yeast, Arxula adeninivorans, Aspergillus niger, Aspergillus niger var. awamori, Aspergillus oryzae, Candida etchellsii, Candida guilliermondii, Candida humilis, Candida lipolytica, Candida pseudotropicalis, Candida utilis, Candida versatilis, Debaryomyces hansenii, Endothia parasitica, Eremothecium ashbyii, Fusarium moniliforme, Hansenula polymorpha, Kluyveromyces lactis, Kluyveromyces marxianus, Kluyveromyces thermotolerans, Morteirella vinaceae var. raffinoseutilizer, Mucor miehei, Mucor miehei var. Cooney et Emerson, Mucor pusillus Lindt, Penicillium roquefortii, Pichia methanolica, Pichia pastoris (Komagataella phaffii), Pichia (Scheffersomyces) stipitis, Rhizopus niveus, Rhodotorula sp., Saccharomyces bayanus, Saccharomyces beticus, Saccharomyces cerevisiae, Saccharomyces chevalieri, Saccharomyces diastaticus, Saccharomyces ellipsoideus, Saccharomyces exiguus, Saccharomyces florentinus, Saccharomyces fragilis, Saccharomyces pastorianus, Saccharomyces pombe, Saccharomyces sake, Saccharomyces uvarum, Sporidiobolus johnsonii, Sporidiobolus salmonicolor, Sporobolomyces roseus, Trichoderma reesi, Xanthophyllomyces dendrorhous, Yarrowia lipolytica, Zygosaccharomyces rouxii, and derivatives and crosses thereof.

Non-limiting examples of suitable microbes include Acetobacter suboxydans, Acetobacter xylinum, Actinoplane missouriensis, Arthrospira platensis, Arthrospira maxima, Bacillus cereus, Bacillus coagulans, Bacillus licheniformis, Bacillus stearothermophilus, Bacillus subtilis, Escherichia coli, Lactobacillus acidophilus, Lactobacillus bulgaricus, Lactobacillus reuteri, Lactococcus lactis, Lactococcus lactis Lancefield Group N, Leuconostoc citrovorum, Leuconostoc dextranicum, Leuconostoc mesenteroides strain NRRL B-512 (F), Micrococcus lysodeikticus, Spirulina, Streptococcus cremoris, Streptococcus lactis, Streptococcus lactis subspecies diacetylactis, Streptococcus thermophilus, Streptomyces chattanoogensis, Streptomyces griseus, Streptomyces natalensis, Streptomyces olivaceus, Streptomyces olivochromogenes, Streptomyces rubiginosus, Xanthomonas campestris, and derivatives and crosses thereof. Additional strains that can be used as recombinant host cells are known in the art. It should be understood that the term “recombinant host cell” is intended to refer not only to the particular subject cell but to the progeny of such a cell. Because certain modifications may occur in succeeding generations due to either mutation or environmental influences, such progeny may not, in fact, be identical to the parent cell, but is still included within the scope of the term “recombinant host cell” as used herein.

In some embodiments, the recombinant host cells comprise genetic modifications that improve production of the recombinant resilins provided herein. Non-limiting examples of such genetic modifications include altered promoters, altered kinase activities, altered protein folding activities, altered protein secretion activities, altered gene expression induction pathways, and altered protease activities.

The recombinant host cells provided herein are generated by transforming cells of suitable origin with vectors provided herein. For such transformation, the vectors can be circularized or be linear. Recombinant host cell transformants comprising the vectors can be readily identified, e.g., by virtue of expressing drug resistance or auxotrophic markers encoded by the vectors that permit selection for or against growth of cells, or by other means (e.g., detection of light emitting peptide comprised in vectors, molecular analysis of individual recombinant host cell colonies, e.g., by restriction enzyme mapping, PCR amplification, or sequence analysis of isolated extrachromosomal vectors or chromosomal integration sites).

In some embodiments, the recombinant host cells provided herein can produce high titers of the recombinant resilins provided herein. In some such embodiments, the recombinant host cells produce the recombinant resilins at a rate of greater than 2 mg resilin/g dry cell weight/hour, 4 mg resilin/g dry cell weight/hour, 6 mg resilin/g dry cell weight/hour, 8 mg resilin/g dry cell weight/hour, 10 mg resilin/g dry cell weight/hour, 12 mg resilin/g dry cell weight/hour, 14 mg resilin/g dry cell weight/hour, 16 mg resilin/g dry cell weight/hour, 18 mg resilin/g dry cell weight/hour, 20 mg resilin/g dry cell weight/hour, 25 mg resilin/g dry cell weight/hour, or 30 mg resilin/g dry cell weight/hour; from 2 to 40, 30, 20, 10, or 5 mg resilin/g dry cell weight/hour; from 5 to 40, 30, 20, or 10 mg resilin/g dry cell weight/hour; from 10 to 40, 30, or 20 mg resilin/g dry cell weight/hour; from 20 to 40, or 30 mg resilin/g dry cell weight/hour; or from 30 to 40 mg resilin/g dry cell weight/hour. In other such embodiments, the recombinant host cells secrete the recombinant resilins at a rate of greater than 2 mg resilin/g dry cell weight/hour, 4 mg resilin/g dry cell weight/hour, 6 mg resilin/g dry cell weight/hour, 8 mg resilin/g dry cell weight/hour, 10 mg resilin/g dry cell weight/hour, 12 mg resilin/g dry cell weight/hour, 14 mg resilin/g dry cell weight/hour, 16 mg resilin/g dry cell weight/hour, 18 mg resilin/g dry cell weight/hour, 20 mg resilin/g dry cell weight/hour, 25 mg resilin/g dry cell weight/hour, or 30 mg resilin/g dry cell weight/hour; from 2 to 40, 30, 20, 10, or 5 mg resilin/g dry cell weight/hour; from 5 to 40, 30, 20, or 10 mg resilin/g dry cell weight/hour; from 10 to 40, 30, or 20 mg resilin/g dry cell weight/hour; from 20 to 40, or 30 mg resilin/g dry cell weight/hour; or from 30 to 40 mg resilin/g dry cell weight/hour. The identities of the recombinant resilins produced can be confirmed by HPLC quantification, Western blot analysis, polyacrylamide gel electrophoresis, and 2-dimensional mass spectroscopy (2D-MS/MS) sequence identification.

In some embodiments, the recombinant host cells provided herein have high secreted fractions of the recombinant resilins provided herein. In some such embodiments, the recombinant host cells have secreted fractions of recombinant resilient that is greater than 50%, 60%, 70%, 80%, or 90%; from 50% to 100%, 90%, 80%, 70%, or 60%; from 60% to 100%, 90%, 80%, or 70%; from 70% to 100%, 90%, or 80%; from 90% to 100%, or 90%; or from 90% to 100%.

Production and secretion of recombinant resilins can be influenced by the number of copies of the secreted resilin coding sequences comprised in the recombinant host cells and/or the rate of transcription of the secreted resilin coding sequences comprised in the recombinant host cells. In some embodiments, the recombinant host cells comprise a single secreted resilin coding sequence. In other embodiments, the recombinant host cells comprise 2 or more (e.g., 3, 4, 5, or more) secreted resilin coding sequences. In some embodiments, the recombinant host cells comprise secreted resilin coding sequences that are operably linked to strong promoters. Non-limiting examples of strong promoters include the pGCW14 promoter of Pichia pastoris. In some embodiments, the recombinant host cells comprise secreted resilin coding sequences that are operably linked to medium promoters. Non-limiting examples of such medium promoters include the pGAP promoter of Pichia pastoris. In some embodiments, the recombinant host cells comprise coding sequences encoding resilins under the control of weak promoters.

The fermentations provided herein comprise recombinant host cells described herein and a culture medium suitable for growing the recombinant host cells.

The fermentations are obtained by culturing the recombinant host cells in culture media that provide nutrients needed by the recombinant host cells for cell survival and/or growth, and for secretion of the recombinant resilins. Such culture media typically contain an excess carbon source. Non-limiting examples of suitable carbon sources include monosaccharides, disaccharides, polysaccharides, and combinations thereof. Non-limiting examples of suitable monosaccharides include glucose, galactose, mannose, fructose, ribose, xylose, arabinose, ribose, and combinations thereof. Non-limiting examples of suitable disaccharides include sucrose, lactose, maltose, trehalose, cellobiose, and combinations thereof. Non-limiting examples of suitable polysaccharides include raffinose, starch, glycogen, glycan, cellulose, chitin, and combinations thereof.

In some embodiments, the fermentations comprise recombinant resilins in amounts of at least 1%, 5%, 10%, 20%, or 30%; from 1% to 100%, 90%, 80%, 70%, 60%, 50%, 40%, 30%, 20%, or 10%; from 10% to 100%, 90%, 80%, 70%, 60%, 50%, 40%, 30%, or 20%; from 20% to 100%, 90%, 80%, 70%, 60%, 50%, 40%, or 30%; from 30% to 100%, 90%, 80%, 70%, 60%, 50%, or 40%; from 40% to 100%, 90%, 80%, 70%, 60%, or 50%; from 50% to 100%, 90%, 80%, 70%, or 60%; from 60% to 100%, 90%, 80%, or 70%; from 70% to 100%, 90%, or 80%; from 80% to 100%, or 90%; or from 90% to 100% by weight of the total fermentation.

In some embodiments, the fermentations comprise recombinant resilin in an amount of at least 2 g/L, 5 g/L, 10 g/L, 15 g/L, 20 g/L, 25 g/L, or 30 g/L; from 2 g/L to 300 g/L, 200 g/L, 100 g/L, 90 g/L, 80 g/L, 70 g/L, 60 g/L, 50 g/L, 40 g/L, 30 g/L, 20 g/L, or 10 g/L; from 10 g/L to 300 g/L, 200 g/L, 100 g/L, 90 g/L, 80 g/L, 70 g/L, 60 g/L, 50 g/L, 40 g/L, 30 g/L, or 20 g/L; from 20 g/L to 300 g/L, 200 g/L, 100 g/L, 90 g/L, 80 g/L, 70 g/L, 60 g/L, 50 g/L, 40 g/L, or 30 g/L; from 30 g/L to 300 g/L, 200 g/L, 100 g/L, 90 g/L, 80 g/L, 70 g/L, 60 g/L, 50 g/L, or 40 g/L; from 40 g/L to 300 g/L, 200 g/L, 100 g/L, 90 g/L, 80 g/L, 70 g/L, 60 g/L, or 50 g/L; from 50 g/L to 300 g/L, 200 g/L, 100 g/L, 90 g/L, 80 g/L, 70 g/L, or 60 g/L; from 60 g/L to 300 g/L, 200 g/L, 100 g/L, 90 g/L, 80 g/L, or 70 g/L; from 70 g/L to 300 g/L, 200 g/L, 100 g/L, 90 g/L, or 80 g/L; from 80 g/L to 300 g/L, 200 g/L, 100 g/L, or 90 g/L; from 90 g/L to 300 g/L, 200 g/L, or 100 g/L; from 100 g/L to 300 g/L, or 200 g/L; or from 200 g/L to 300 g/L.

### Methods of Producing Recombinant Resilin

Further provided herein are methods for the production of the recombinant resilins described herein.

The methods are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y., 1989; Ausubel et al., Current Protocols in Molecular Biology, Greene Publishing Associates, 1992, and Supplements to 2002); Harlow and Lane, Antibodies: A Laboratory Manual, Cold Spring Harbor Laboratory Press, Cold Spring Harbor, N.Y., 1990; Taylor and Drickamer, Introduction to Glycobiology, Oxford Univ. Press, 2003; Worthington Enzyme Manual, Worthington Biochemical Corp., Freehold, N.J.; Handbook of Biochemistry: Section A Proteins, Vol I, CRC Press, 1976; Handbook of Biochemistry: Section A Proteins, Vol II, CRC Press, 1976; Essentials of Glycobiology, Cold Spring Harbor Laboratory Press, 1999.

In some embodiments, a novel method is utilized to secrete resilin extracellularly from a host cell. In some embodiments, the method comprises constructing a vector comprising a secreted resilin coding sequence (step 1001 in FIG. 2), transforming the vector into a host cell (step 1002 in FIG. 2), and then culturing the recombinant host cells to secrete resilin extracellularly (step 1003 in FIG. 2). In some embodiments, the method includes secreting the resilin extracellularly at a rate greater than 2 mg resilin/g dry cell weight/hour, 4 mg resilin/g dry cell weight/hour, 6 mg resilin/g dry cell weight/hour, 8 mg resilin/g dry cell weight/hour, 10 mg resilin/g dry cell weight/hour, 12 mg resilin/g dry cell weight/hour, 14 mg resilin/g dry cell weight/hour, 16 mg resilin/g dry cell weight/hour, 18 mg resilin/g dry cell weight/hour, 20 mg resilin/g dry cell weight/hour, 25 mg resilin/g dry cell weight/hour, or 30 mg resilin/g dry cell weight/hour; from 2 to 40, 30, 20, 10, or 5 mg resilin/g dry cell weight/hour; from 5 to 40, 30, 20, or 10 mg resilin/g dry cell weight/hour; from 10 to 40, 30, or 20 mg resilin/g dry cell weight/hour; from 20 to 40, or 30 mg resilin/g dry cell weight/hour; or from 30 to 40 mg resilin/g dry cell weight/hour. In some embodiments, the secreted resilin is then purified (step 1004 in FIG. 2), and the purified resilin is cross-linked to form an elastomer (step 1005 in FIG. 2). In some embodiments, the methods provided herein comprise the step of transforming cells with vectors provided herein to obtain recombinant host cells provided herein (step 1002 in FIG. 2). Methods for transforming cells with vectors are well-known in the art. Non-limiting examples of such methods include calcium phosphate transfection, dendrimer transfection, liposome transfection (e.g., cationic liposome transfection), cationic polymer transfection, electroporation, cell squeezing, sonoporation, optical transfection, protoplast fusion, impalefection, hyrodynamic delivery, gene gun, magnetofection, and viral transduction. One skilled in the art is able to select one or more suitable methods for transforming cells with vectors provided herein based on the knowledge in the art that certain techniques for introducing vectors work better for certain types of cells.

In some embodiments, the methods further comprise the step of culturing the recombinant host cells provided herein in culture media under conditions suitable for obtaining the fermentations provided herein (step 1003 in FIG. 2). In some embodiments, the conditions and culture media are suitable to facilitate secretion of the recombinant proteins from the recombinant host cells into the culture media. Suitable culture media for use in these methods are known in the art, as are suitable culture conditions. Exemplary details of culturing yeast host cells are described in Idiris et al., Appl. Microbiol. Biotechnol. 86:403-417, 2010; Zhang et al., Biotechnol. Bioprocess. Eng. 5:275-287, 2000; Zhu, Biotechnol. Adv. 30:1158-1170, 2012; Li et al., MAbs 2:466-477, 2010.

In some embodiments, the methods further comprise the step of purifying secreted recombinant resilins from the fermentations provided herein to obtain the recombinant resilins provided herein (step 1004 in FIG. 2). Purification can occur by a variety of methods known in the art for purifying secreted proteins from fermentations. Common steps in such methods include centrifugation (to remove cells) followed by precipitation of the proteins using precipitants or other suitable cosmotropes (e.g., ammonium sulfate). The precipitated protein can then be separated from the supernatant by centrifugation, and resuspended in a solvent (e.g., phosphate buffered saline [PBS]). The suspended protein can be dialyzed to remove the dissolved salts. Additionally, the dialyzed protein can be heated to denature other proteins, and the denatured proteins can be removed by centrifugation. Optionally, the purified recombinant resilins can be coacervated.

In various embodiments, methods of purifying the secreted recombinant proteins from the fermentation can include various centrifugation steps in conjunction with solubilizing protein in a whole cell broth or cell pellet with known chaotropes such as urea or guanidine thiocyanate.

In some embodiments, recombinant resilin protein can be purified by centrifuging a whole cell broth to produce a first pellet of cells and a first supernatant, and extracting the first supernatant to produce a clear cell broth. The first supernatant is then precipitated using ammonium sulfate and centrifuged to produce a second pellet and second supernatant which was discarded. The second pellet is then re-suspended in PBS for dialysis. In some embodiments, the dialyzed solution is then subject to high temperature to denature proteins that are less stable than the target recombinant protein. Then, denatured proteins are removed by centrifuging the dialyzed and denatured solution to produce a third pellet and third supernatant. The third supernatant is retained from the denatured solution, then coacervated by chilling the third supernatant to induce a phase separation into a dense lower layer containing the target recombinant resilin protein and an upper layer. Samples purified by this method can be referred to as “CCB” samples. In some embodiments, multiple coacervations are performed by retaining the lower layer and incubating the lower layer at a lower temperature to induce further phase separation.

In some embodiments, recombinant resilin protein can be purified by centrifuging a whole cell broth to produce a first pellet of cells and protein proximal to the cells (e.g. adherent to the cells, on the surface of the cells) and/or insoluble protein (e.g. protein aggregates) and first supernatant, then discarding the first supernatant to obtain the first pellet. The first pellet can then be re-suspended in guanidine thiocyanate to solubilize recombinant resilin protein. The re-suspension is then centrifuged again produce a second pellet and a second supernatant. The second supernatant is then dialyzed against PBS and subject to high temperature in order to denature proteins other than recombinant resilin and centrifuged to produce a third pellet and third supernatant. In some embodiments, the third supernatant is then subject to coacervation by chilling to yield phase separation into a dense lower layer containing recombinant resilin and an upper layer. Samples purified by this method can be referred to as “gel layer” samples. In some embodiments, multiple coacervations are performed by retaining the lower layer and incubating the lower layer at a lower temperature to induce further phase separation.

In some embodiments, recombinant resilin protein can be purified by centrifuging a whole cell broth to produce a pellet and supernatant, then discarding the supernatant to obtain a pellet of cells and protein proximal to the cells (e.g. adherent to the cells, on the surface of the cells) and/or insoluble protein (e.g. protein aggregates). The pellet of cells is then re-suspended in guanidine thiocyanate to solubilize the protein that was proximal to the cells. The re-suspension is centrifuged again produce a second pellet of cells and a second supernatant. The second supernatant is then precipitated with ammonium sulfate and centrifuged to produce a third pellet and third supernatant. The third pellet is then suspended in guanidine thiocyanate, then dialyzed against PBS and subject to high temperature to denature proteins other than recombinant resilin and centrifuged to produce a fourth supernatant and fourth pellet. The fourth supernatant is then subject to coacervation by chilling to yield phase separation. Samples purified by this method can be referred to as “gel layer precipitated” samples.

In some embodiments, recombinant resilin protein can be purified by adding urea to a whole cell broth to solubilize the protein, then centrifuging the whole cell broth to produce a first pellet and first supernatant. The first supernatant is then precipitated using ammonium sulfate and centrifuged to produce a second pellet and second supernatant. The second supernatant is discarded and the second pellet is then re-suspended in guanidine thiocyanate and dialyzed against PBS, then subject to high temperature in order to denature proteins other than recombinant resilin and centrifuged again to produce a third pellet and a third supernatant. The third supernatant is then coacervated by chilling the third supernatant to induce a phase separation into a dense lower layer containing recombinant resilin and an upper layer. Samples purified by this method can be referred to as “Urea WCBE” samples.

In some embodiments, recombinant resilin protein can be purified by centrifuging a whole cell broth to produce a first pellet and first supernatant, then discarding the first supernatant to obtain a first pellet of cells and protein proximal to the cells (e.g. adherent to the cells, on the surface of the cells) and/or insoluble protein (e.g. protein aggregates). The first pellet of cells is then re-suspended in guanidine thiocyanate to solubilize the protein. The re-suspension is centrifuged again to produce a second pellet of cells and a second supernatant. The second supernatant is then dialyzed against PBS and then centrifuged to produce a heavy phase of protein, a light phase of supernatant and a film separating the heavy phase from the light phase. The heavy phase of protein is then isolated by discarding the light phase and the film. Samples purified by this method can be referred to as “Dense layer” samples.

In some embodiments, the recombinant resilin composition is a foam material. In some embodiments, a method of preparing the recombinant resilin foam, comprises: providing a cross-linked recombinant resilin solid composition in an aqueous solvent; exchanging said aqueous solvent with a polar nonaqueous solvent; and introducing one or more bubbles to the cross-linked recombinant resilin solid composition. Any method of introducing bubbles known in the art may be used herein. For instance, methods of introducing bubbles include, but are not limited to, vortexing, mixing, adding yeast, and chemical reactions. In some embodiments, the introducing the one or more bubbles may occur at the same time the cross-linked recombinant resilin solid composition is provided. In some embodiments, the introducing the one or more bubbles may occur after the cross-linked recombinant resilin solid composition is provided.

Blowing agents typically are introduced into polymeric material to make polymer foams. According to one embodiment, a chemical blowing agent is mixed with a polymer. The chemical blowing agent undergoes a chemical reaction in the polymeric material, typically under conditions in which the polymer is molten, causing formation of a gas.

Chemical blowing agents generally are low molecular weight organic compounds that decompose at a particular temperature and release a gas such as nitrogen, carbon dioxide, or carbon monoxide.

Exemplary chemical blowing agents include, but are not limited to, sodium bicarbonate, potassium bicarbonate, ammonium, azodicarbonamide, isocyanate, hydrazine, isopropanol, 5-phenyltetrazole, triazole, 4,4′oxybis(benzenesulfonyl hydrazide) (OBSH), trihydrazine triazine (THT), hydrogen phosphate, tartaric acid, citric acid, and toluenesulphonyl semicarbazide (TSS).

In some embodiments, foaming agents, thickeners, and/or hardeners are added to the recombinant resilin solid. Exemplary foaming agents include, but are not limited to, xanthan gum, sodium dodecyl sulfate, ammonium lauryl sulfate, bovine serum albumin. Exemplary thickeners include, but are not limited to, fumed silica and xanthan gum. Exemplary hardeners include, but are not limited to, aliphatic polyamine, fatty polyamides, aromatic polyamine hardeners, anhydride hardeners, boron trifluoride hardeners, and curing agents (dicyandiamide).

According to another embodiment, a physical blowing agent, i.e., a fluid that is a gas under ambient conditions, is injected into a molten polymeric stream to form a mixture. The mixture is subjected to a pressure drop, causing the blowing agent to expand and form bubbles (cells) in the polymer. In some embodiments, the pressure required is about 500 psi to about 2000 psi, e.g., about 600 psi to about 1000 psi, about 700 psi to about 1500 psi, and about 800 psi to about 2000 psi. In some embodiments, the pressure required is about 500 psi.

Exemplary physical blowing agents include, but are not limited to, chlorofluorocarbon (CFC), dissolved nitrogen, N₂, CH₄, H₂, CO₂, Ar, pentane, isopentane, hexane, methylene dichloride, and dichlorotetra-fluoroethane.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3rd Ed. (Plenum Press) Vols A and B (1992).

### Example 1: Production of Recombinant Resilin

Pichia pastoris recombinant host cells that secrete recombinant resilin were generated by transforming a HIS+derivative of GS115 (NRRL Y15851) Pichia pastoris (Komagataella phaffii) with vectors comprising secreted resilin coding sequences.

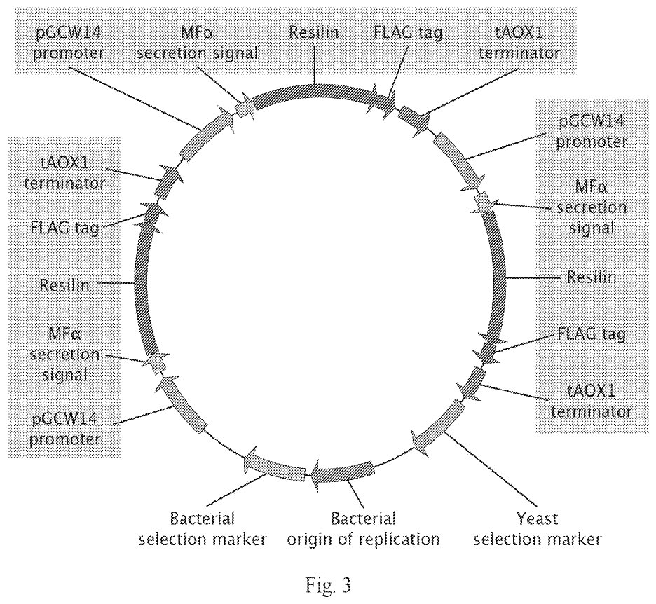

The vectors each comprised 3 resilin coding sequences fused in frame to an N-terminal secretion signal (alpha mating factor leader and pro sequence), and in some instances a C-terminal 3×FLAG tag (SEQ ID NO: 45) (see FIG. 3). Each of the secreted resilin coding sequences was flanked by a promoter (pGCW14) and a terminator (tAOX1 pA signal). The vectors further comprised a targeting region that can direct integration of the 3 secreted resilin coding sequences to the HSP82 locus of the Pichia pastoris genome, dominant resistance markers for selection of bacterial and yeast transformants, as well as a bacterial origin of replication.

The resilin coding sequences were obtained from scientific literature and from searching public sequence databases. The nucleotide sequences were translated into amino acid sequences and then codon-optimized. Both full length and truncated resilin sequences were chosen. Selected secreted resilin coding sequences are listed in Table 1.

TABLE 1
Exemplary full-length and truncated resilin amino acid sequences and recombinant host strains

| | | | Amino | | | | |
|---|---|---|---|---|---|---|---|
| | | | Acid | | | | |
| | | Short | SEQ | With FLAG tag | | Without FLAG tag | |
| Species | Type | Name | ID NO: | Plasmid | Strain | Plasmid | Strain |
| Drosophila | Full length | Ds_ACB | 1 | RMp4830 | RMs1209 | RMp4842 | RMs1221 |
| sechellia | | | | | | | |
| Drosophila | A repeats + | Ds_AC | 2 | RMp4831 | RMs1210 | RMp4843 | RMs1222 |
| sechellia | Chitin | | | | | | |
| | binding | | | | | | |
| | domain | | | | | | |
| Drosophila | A repeats | Ds_A | 3 | RMp4832 | RMs1211 | RMp4844 | RMs1223 |
| sechellia | only | | | | | | |
| Acromyrmex | A repeats | Ae_A | 4 | RMp4833 | RMs1212 | RMp4845 | RMs1224 |
| echinatior | only | | | | | | |
| Aeshna sp. | B repeats | As_B | 5 | RMp4834 | RMs1213 | RMp4846 | RMs1225 |
| | only | | | | | | |
| Aeshna sp. | Full length | As_ACB | 6 | RMp4835 | RMs1214 | RMp4847 | RMs1226 |
| Haematobia | A repeats | Hi_A | 7 | RMp4836 | RMs1215 | RMp4848 | RMs1227 |
| irritans | only | | | | | | |
| Haematobia | Full length | Hi_ACB | 8 | RMp4837 | RMs1216 | RMp4849 | RMs1228 |
| irritans | | | | | | | |
| Ctenocephalides | A repeats | Cf_A | 9 | RMp4838 | RMs1217 | RMp4850 | RMs1229 |
| felis | only | | | | | | |
| Ctenocephalides | B repeats | Cf_B | 10 | RMp4839 | RMs1218 | RMp4851 | RMs1230 |
| felis | only | | | | | | |
| Bombus | A repeats | Bt_A | 11 | RMp4840 | RMs1219 | RMp4852 | RMs1231 |
| terrestris | only | | | | | | |
| Tribolium | A repeats | Tc_A | 12 | RMp4841 | RMs1220 | RMp4853 | RMs1232 |
| castaneum | only | | | | | | |

The vectors were transformed into Pichia pastoris using electroporation to generate host strains comprising 3 integrated copies of each secreted resilin coding sequence. Transformants were plated on YPD agar plates supplemented with an antibiotic, and incubated for 48 hours at 30° C.

Clones from each final transformation were inoculated into 400 μL of Buffered Glycerol-complex Medium (BMGY) in 96-well blocks, and incubated for 24 hours at 30° C. with agitation at 1,000 rpm. A sample was removed, the recombinant host cells were pelleted via centrifugation, and the supernatant was recovered and run on a SDS-PAGE gel for analysis of resilin content via Coomassie gel and Western blot analysis (for polypeptides comprising the 3×FLAG tag). For FLAG-tagged proteins, the remaining cultures were used to inoculate minimal media cultures in duplicate for ELISA measurements. One duplicate was pelleted and the supernatant was measured directly. The second duplicate was extracted with guanidine thiocyanate and both the intra- and extra-cellular fractions were measured.

Recombinant resilin from numerous species expressed successfully in the Pichia pastoris recombinant host cells. Recombinant host cells secreted up to 90% of the recombinant resilin produced.

SEQ ID NO: 49 of the sequence listing provides the full-length Drosophila sechellia resilin sequence (Ds_ACB) that is expressed along with signal sequences that are later cleaved. The first sequence (italics), is an alpha mating factor precursor protein signal sequence (SEQ ID NO: 46) that is cleaved twice after transcription by a signal peptidase followed by cleavage with Kex2. The second sequence (bold) is an EAEA repeat (SEQ ID NO: 47) that is cleaved by Ste13 (SEQ ID NO: 47). The third sequence (lower case) corresponds to Drosophila sechellia full-length resilin (SEQ ID NO 1). The fourth sequence (bold and italicized) corresponds to a linker sequence (SEQ ID NO: 46). The fifth sequence (underlined) corresponds to the 3× FLAG tag (SEQ ID NO: 45).

Edman sequencing confirmed that the N-terminus of the protein sequences at the approximately 110 kDa band corresponded to the full-length length Drosophila sechellia resilin sequence. Specifically, the N-terminus sequencing showed that the N-terminus either corresponded to “EAEA” (SEQ ID NO: 47) or “GRPE” (SEQ ID NO: 63), respectively the full-length Drosophila sechellia resilin sequence with or without the EAEA repeat (SEQ ID NO: 47).

### Example 2: Purification of Recombinant Resilin

Non-FLAG-tagged Ds_ACB and Ae_A recombinant resilin polypeptides were purified as follows: Strains RMs1221 (expressing Ds_ACB) and RMs1224 (expressing Ae_A) were grown in 500 mL of BMGY in flasks for 48 hours at 30° C. with agitation at 300 rpm.

The protocol for purification was adapted from Lyons et al. (2007). Cells were pelleted by centrifugation, and supernatants were collected. Proteins were precipitated by addition of ammonium sulfate. The precipitated proteins were resuspended in a small volume of phosphate buffered saline (PBS), and the resuspended samples were dialyzed against PBS to remove salts. The dialyzed samples were then heated to denature native proteins, and denatured proteins were removed by centrifugation. The retained supernatants contained the purified resilin polypeptides. Optionally, the retained supernatants were chilled, which caused coacervation, resulting in a concentrated lower phase and dilute upper phase.

A second set of samples was prepared by centrifuging a whole cell broth to produce a first pellet of cells and protein proximal to the cells (e.g. adherent to the cells, on the surface of the cells) and/or insoluble protein (e.g. protein aggregates) and first supernatant, then discarding the first supernatant to obtain the first pellet. The first pellet was re-suspended in guanidine thiocyanate to solubilize Ds_ACB. The re-suspension was centrifuged again produce a second pellet and a second supernatant. The second supernatant was then dialyzed against PBS and subject to high temperature in order to denature proteins other than Ds_ACB and centrifuged to produce a third pellet and third supernatant. The third supernatant was subject to coacervation by chilling to yield phase separation into a dense lower layer containing Ds_ACB and an upper layer. This is also referred to herein as gel layer separation, or coacervation samples. In some gel layer samples, multiple coacervations were performed by retaining the lower layer and incubating the lower layer at a lower temperature to induce further phase separation.

TABLE 2
Stability of Cross-linked Resilin

| Sample | 110 kDa band? | Degradation | Time as solid | |
|---|---|---|---|---|
| A | Yes | Substantial | 13 | days |
| B | No | Substantial | 8 | days |
| C | No | Substantial | 6 | days |
| D | No | Substantial | 6 | days |
| E | Yes | Minimal | N/A | |
| F | Yes | Minimal | 27 | days |
| G | No | Substantial | 8 | days |
| H | Yes | Minimal | 15 | days |
| I | Yes | Substantial | 15 | days |
| J | No | Substantial | 6 | days |
| K | Yes | Minimal | 13 | days |
| L | Yes | Minimal | 13 | days |

### Example 3: Cross-Linking of Purified, Secreted, Recombinant Resilin

Purified resilin was cross-linked via one of three methods: photo cross-linking (adapted from Elvin et al. 2005), enzymatic cross-linking (adapted from Qin et al. 2009), or the method described herein (i.e., exposure to ammonium persulfate and heat).

### Photo Cross-Linking

For photo cross-linking, resilin protein was mixed with ammonium persulfate and tris (bipyridine) ruthenium (II) ([Ru(bpy)3]2+). The mixture was exposed to bright white light, after which the mixture formed a rubbery solid.

Specifically, a 10 mM Ru (II) photocatalyst (CAS 50525-27-4) in water and 100 mM ammonium persulfate in water stock solutions were prepared. Purified resilin powder was dissolved in phosphate buffered saline to a concentration between 20-30 wt % resilin to prepare the resilin stock solution.

2 μL of the 10 mM Ru (II) photocatalyst solution and 2 μL of the 100 mM ammonium persulfate was added to and mixed with 10 μL of resilin stock solution to form the photo cross-linking solution. Bubbles were removed from the photo cross-linking solution by centrifugation, and the photo cross-linking solution was poured into a mold.

The photo cross-linking solution was then exposed to white light (White LED floodlight, Zochlon USPT100W 110V) for 30 seconds to 1 minute.

### Enzymatic Cross-Linking

For enzymatic cross-linking, resilin protein was mixed with horseradish peroxidase (HRP) and hydrogen peroxide. The mixture was incubated at 37° C., after which the mixture formed a rubbery solid.

Specifically, a 10 mg/mL stock of horse radish peroxidase (type 1, P8125 Sigma) in water (HRP stock) and a 100 mM H₂O₂ stock in water. Purified resilin powder was dissolved in phosphate buffered saline to a concentration between 20-30 wt % resilin to prepare the resilin stock solution.

6 μL of HRP stock was added to and mixed with 21 μL of the resilin stock solution. 3 uL H₂O₂ stock was then added and mixed by vortexing to form the enzymatic cross-linking solution. Bubbles were removed from the enzymatic cross-linking solution by centrifugation, and the enzymatic cross-linking solution was poured into a mold while maintaining the solution at 4° C. To perform cross-linking, the enzymatic cross-linking solution was incubated for 15 minutes at 37° C. The resulting cross-linked resilin composition includes an active enzyme covalently bound to the resilin solid.

### Persulfate and Heat Cross-Linking

Purified resilin was cross-linked using ammonium persulfate and heat.

Specifically, purified resilin powder was dissolved in phosphate buffered saline to a concentration between 20-30 wt % resilin to prepare the resilin stock solution. 4 μL of n 200 mM ammonium persulfate in water was mixed with 18 μL of the resilin stock solution to a final concentration of 36 mM ammonium persulfate. Bubbles were removed from the persulfate cross-linking solution by centrifugation, and the persulfate cross-linking solution was poured into a mold.

To perform cross-linking, the persulfate cross-linking solution was incubated at 80° C. for 2.5 hours in a sealed humid environment. The resulting cross-linked resilin composition does not leave an active catalyst behind in the resilin solid and results in a composition with a less degraded protein.

### Degradation of HRP Cross-Linked Compositions

The stability of the cross-linked samples was assessed over time by determining the duration each cross-linked samples remained a solid through daily observation. Table 3 shows the time as a solid for each cross-linked sample prepared via photo cross-linking (Ruthv), enzymatic cross-linking (HRP), and cross-linking via ammonium persulfate and heat (AP+heat). As shown in Table 3, at room temperature, the resilin compositions cross-linked via photo cross-linking or via ammonium persulfate had a much longer duration of stability than resilin compositions cross-linked via enzymatic cross-linking.

TABLE 3
Degradation of resilin compositions cross-linked
by different cross-linking procedures

| | | | Incubation | Observed Melt |
|---|---|---|---|---|
| Resilin # | Crosslinking | Washing? | Temp. (° C.) | Time (days) |
| 1967 I-8 | HRP | N | RT | 8 |
| 1967 I-8 | HRP | N | RT | 8 |
| 1729 I-10 | HRP | N | RT | 8 |
| 1729 I-10 | HRP | N | RT | 10 |
| 1967 I-8 | Ru + hv | N | RT | >92 |
| 1967 I-8 | Ru + hv | N | RT | >92 |
| 1729 I-10 | AP + heat | N | RT | >91 |
| 1967 I-8 | AP + heat | N | RT | >91 |

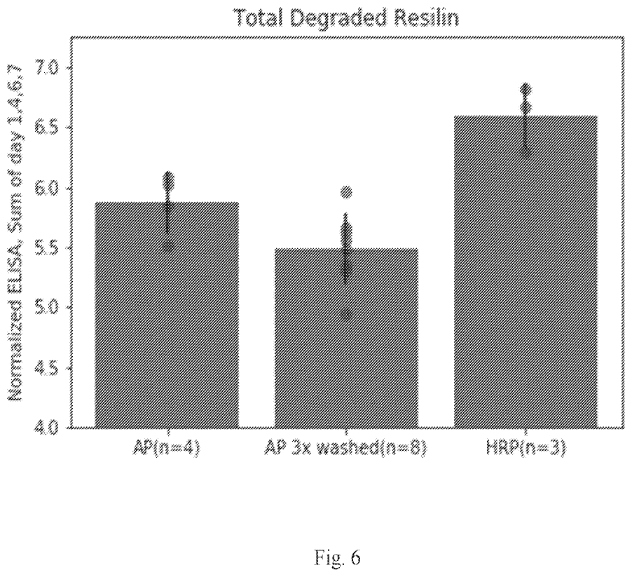

The total degraded resilin from ammonium persulfate cross-linked resilin compositions (AP) and enzymatically cross-linked resilin compositions (HRP) as measured by ELISA is shown in FIG. 6. Increased degradation and secretion of resilin fragments from cross-linked resilin compositions is observed in the HRP samples as compared to AP samples.

This data shows that ammonium persulfate cross-linking is preferred over enzymatic cross-linking for preparation of stable resilin compositions. For enzymatically cross-linked resilin compositions, the cross-linking enzyme remains in the composition and results in degradation of the composition.

### Example 4: Stability of Cross-Linked Full-Length Resilin Vs. Resilin Mixture

Resilin samples generated with varying levels of degradation products and full-length resilin were subject to enzymatic cross-linking as described above. The stability of the cross-linked samples was assessed over time by determining the duration each cross-linked samples remained a solid through daily observation. Table 4 shows the time as a solid for each cross-linked sample. As shown in Table 4, samples comprising full-length resilin had a longer duration of stability than the samples that did not comprise full-length resilin.

TABLE 4
Stability of cross-linked resilin

| Sample | 110 kDa band? | Degradation | Time as solid | |
|---|---|---|---|---|
| A | Yes | Substantial | 13 | days |
| B | No | Substantial | 8 | days |
| C | No | Substantial | 6 | days |
| D | No | Substantial | 6 | days |
| E | Yes | Minimal | N/A | |
| F | Yes | Minimal | 27 | days |
| G | No | Substantial | 8 | days |
| H | Yes | Minimal | 15 | days |
| I | Yes | Substantial | 15 | days |
| J | No | Substantial | 6 | days |
| K | Yes | Minimal | 13 | days |
| L | Yes | Minimal | 13 | days |

### Example 5: Resilin Foam

Crosslinking resilin in the presence of a thickening additive, e.g., fumed silica, and sodium bicarbonate resulted in a foamed resilin with a relatively uniform distribution of bubbles. Altering the amount of fumed silica impacted average bubble size, giving us a lever to tune foamed resilin solids. The results are shown in Table 5.

TABLE 5
Data on resilin foam

| Property | | |
|---|---|---|
| Diameter of bubbles of resilin foam | 0.2-2 | mm |
| with 5 wt % fumed silica (mm) | | |
| Diameter of bubbles of resilin foam | 0.05-0.2 | mm |
| with 10 wt % fumed silica (mm) | | |
| Amount of sodium bicarbonate (% wt) | 0.2-2% | wt |
| Amount of thickener (% wt) | 0-10% | wt |

A solution of resilin was prepared. 0.81 g of resilin was dissolved in 2.19 mL PBS at pH of 7.4. A foaming agent was added and the solution was vortexed to introduce bubbles and crosslinked via heat or light. Fifty mg of xantham gum was added to the solution followed by 0.65 mL of 225 mM ammonium persulfate. Xanthum gum was used as the foaming agent because it also increased viscosity which aided in trapping the bubbles in solution. The solution was then vortexed and heated at 80° C. for 3.5 h.

In some samples, ruthenium (II) and white light were used to crosslink the resilin. In such experiments, 0.405 g resilin lot 3 was dissolved in 1.10 mL PBS. Then, 0.3 mL of 100 mM ammonium persulfate and 0.3 mL of 10 mM Ru (II) catalyst. In other samples, 0.405 g resilin lot 3 was dissolved in 1.10 mL PBS. Then, 25 mg of xantham gum was added, followed by 0.3 mL of 100 mM ammonium persulfate and 0.3 mL of 10 mM Ru (II) catalyst.

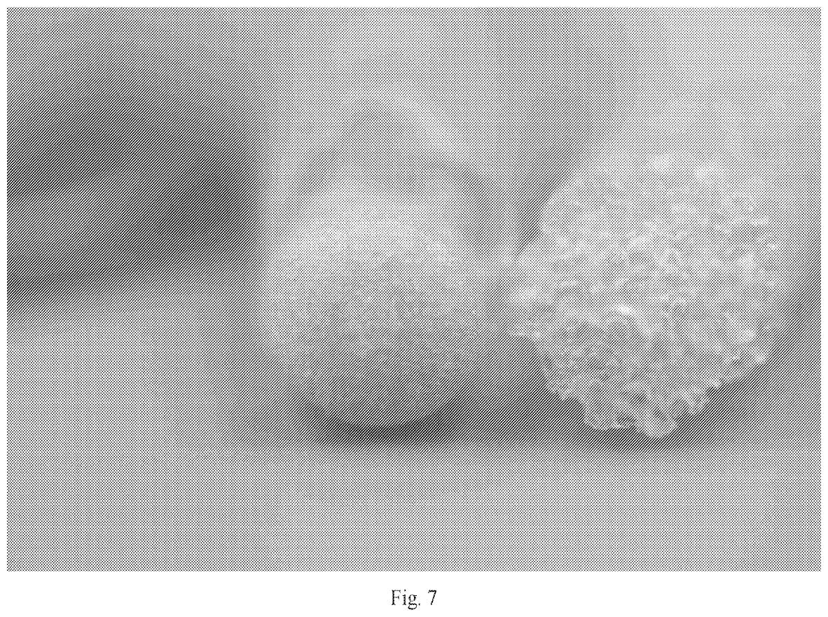

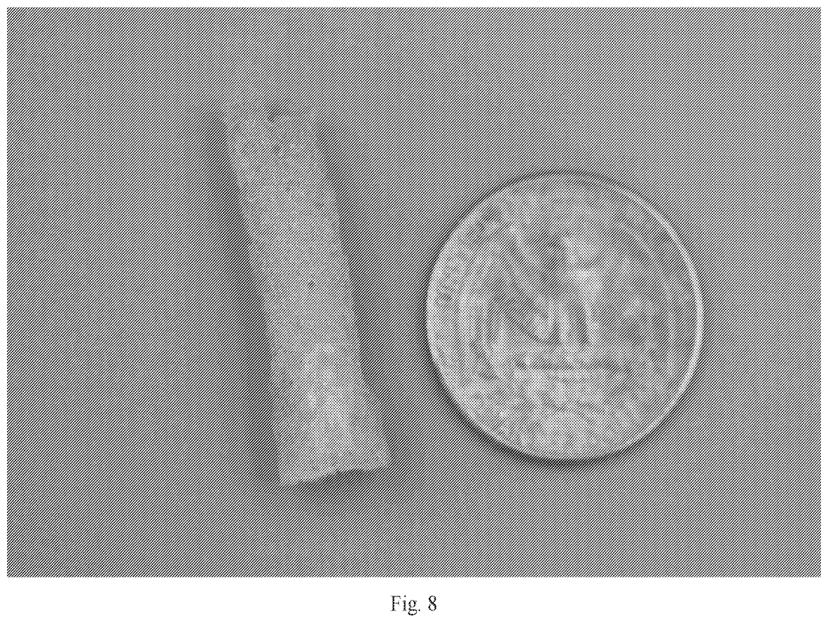

In some samples, sodium bicarbonate was used as a chemical blowing agent. Sodium bicarbonate was added to the resilin solution and crosslinked. A thickener, fumed silica, was added in some sampls. Too much sodium bicarbonate (33 mg/mL or more) inhibited gelation. Using between 6-20 mg/mL of sodium bicarbonate to resilin solution resulted in resilin solids after 3.5 hours at 80° C. with large bubbles. Adding a thickener, fumed silica, to between 4-10 wt %, resulted in a resilin foam with an even distribution of bubbles (FIGS. 7 and 8). Too little fumed silica resulted in a half-foamed resilin solid and adding more than 5 wt % fumed silica resulted in a foamed resilin with smaller bubbles (FIG. 7). The diameter of bubbles of resilin foam with 5 wt % fumed silica was 0.2 to 2 mm. The diameter of bubbles of resilin foam with 10 wt % fumed silica was 0.05 to 0.2 mm.

Specifically, 0.27 g of resilin lot 3 was dissolved in 0.73 mL PBS at pH of 7.4. Then, 37, 18, 10 or 6 mg of sodium bicarbonate was added to the solution followed by 0.22 mL of 225 mM ammonium persulfate and between 0-10 wt % fumed silica. The solution was vortexed and heated at 80° C. for 3.5 h. Experiments were performed in a capped 2 mL tube.

In some samples, an ISCO pump was used to introduce dissolved nitrogen at either 1600 psi or 500 psi while crosslinking the resilin at 83° C. Crosslinking at these pressures proceeded at a slower rate than crosslinking at atmospheric pressure, likely due to the increased amounts of dissolved oxygen.

Specifically, 1.1 mL solution of 225 or 550 mM ammonium persulfate in water was added to a 5 mL solution of 27 wt % resilin in PBS. This solution was then centrifuged for 5 min at 7197 rcf and added to the ISCO pump. The pump was then hooked up to house nitrogen and the ISCO pump was set to its maximum volume of 266 mL, purged with nitrogen for a 3 minutes and then sealed. Then, the pump was either set to 500 psi or 1600 psi and heated for between 2-6 h at 83° C. After releasing the pressure, the resilin was heated for an additional 1-2 hours at 83° C.

### Example 6: Solvent Exchange and Comparison of Cross-Linked Resilin in Aqueous Solvent Vs. Polar Nonaqueous Solvent

As described in detail below, a cross-linked mixture of recombinant resilin in an aqueous solvent was treated with a solvent exchange to replace the solvent with a polar nonaqueous solvent to form a composition with material properties better suited for certain applications.

A resilin powder comprising 19 mass % full-length resilin as measured via SEC (size exclusion chromatography), with the rest of the powder comprising partially degraded resilin, was prepared.

The resilin powder was then dissolved in 1× phosphate buffered saline (PBS), the reported weight percent of resilin is weight by weight (w/w). A 1 g solution of 27 wt % resilin is prepared by adding 0.27 g of resilin to 0.73 g of PBS solution. The resilin solution was then cross-linked in a mold to form a water-based resilin solid composition. The resilin solid formed was a 15 mm diameter×5 mm height disc.

To generate a glycerol-based resilin solid, the water-based solid is exchanged in 20× its volume of 60% glycerol (60:40 glycerol:water (v:v)) at 60 deg C. in a closed container for 16 h. Then, the solid is exchanged in 20× its volume of glycerol at 60 deg C. in an unsealed container for 16 h.

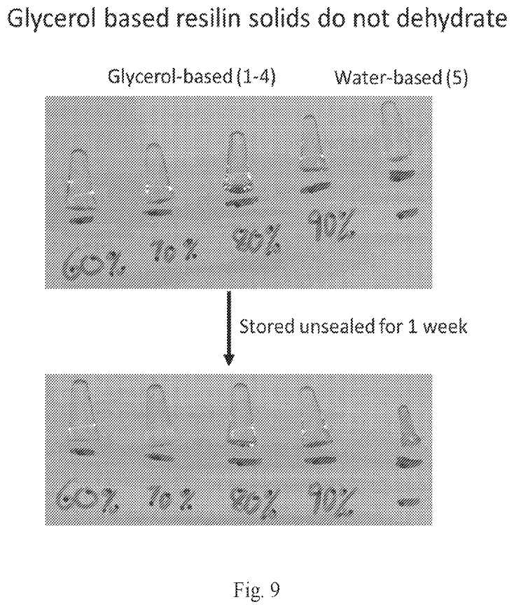

Samples of water-based and glycerol-based resilin solids were stored unsealed for 1 week at room temperature. As shown in FIG. 9, water-based resilin solids dehydrated after only 1 week, while glycerol-based resilin solids retained their original shape without any visible degradation or dehydration.

### Example 7: Solvent Exchange and Comparison of Properties of Multiple Polar Nonaqueous Resilin Solvents

Resilin solids in polar nonaqueous solvents were prepared from cross-linked resilin in 1× phosphate buffered saline (PBS) as follows:

Resilin solid in propylene glycol solvent was prepared by exchanging the resilin solid in 20× its volume of propylene glycol at 60 deg C. in an unsealed container for 16 h or for 2 days (i.e., 2 d. inc.).

Resilin solid in 60-100% glycerol solvent was prepared by exchanging the resilin solid in 20× its volume of 60% glycerol (60:40 glycerol:water (v:v)) at 60 deg C. in a closed container for 16 h. Then, the solid is exchanged in 20× its volume of glycerol at 60 deg C. in an unsealed container for 16 h.

Resilin solid in 100% glycerol solvent was prepared by exchanging the resilin solid in 20× its volume of glycerol at 60 deg C. in an unsealed container for 16 h or for 2 days (i.e., 2 d. inc.).

Resilin solid in ethylene glycol solvent was prepared by exchanging the resilin solid in 20× its volume of ethylene glycol at 60 deg C. in an unsealed container for 16 h.

Resilin solid in propylene glycol solvent was prepared by exchanging the resilin solid in 20× its volume of propylene glycol at 60 deg C. in an unsealed container for 16 h.

To form a resilin solid based on a purification via coacervation, a 27 wt % (w/w) resilin solution was incubated at −5 deg C. and a liquid-liquid phase separation occurred. The bottom coacervate layer was isolated and ammonium persulfate (APS) powder was mixed into the coacervate to a final concentration of 36 mM APS. This solution was then crosslinked at 80 deg C. for 2.5 h. The solid was then exchanged following the typical solvent exchange procedure.

Unless otherwise stated, the resilin powder used is 19 mass % full-length as measured via size-exclusion chromatography (SEC) and the rest of the powder is composed of partially degraded resilin. The 27 wt % “full-length resilin” sample was prepared from resilin powder that is 87 mass % full-length monomeric resilin as measured via SEC.

To characterize the relative amount of the predominant species of resilin in a resilin composition using SEC, resilin powder was dissolved in 5M Guanidine Thiocyanate and injected onto a Yarra SEC-3000 SEC-HPLC column to separate constituents on the basis of molecular weight. Refractive index was used as the detection modality. BSA was used as a general protein standard with the assumption that >90% of all proteins demonstrate dn/dc values (the response factor of refractive index) within ˜7% of each other. Poly(ethylene oxide) was used as a retention time standard, and a BSA calibrator was used as a check standard to ensure consistent performance of the method. A range corresponding to resilin was assessed from 60-40 kDa; higher peaks observed which may correspond to aggregate or polymerized resilin were not included in this quantification. The relative percentage of this resilin peak was reported as mass % and area % to determine the amount of full length resilin in a composition as a portion of total resilin.

A Malvern Kinexus Rheometer RNX2110 was then used to generate stress strain curves for each of the compositions produced as described above. The following protocol was applied at a temperature of 22 deg C.: Step 1: Initial Gap-6.5 mm; Step 2: Descent to final Gap. Steps 1 and 2 were repeated 5×. Values of elastic modulus as measured by the rheometer are relative only, and cannot be compared to materials tested under different conditions or with different instruments.

Stress-strain curves were generated plotting engineering stress vs. engineering strain. Engineering stress is calculated by the following formula: Engineering stress=(Normal Force (N)/A (mm²) of resilin solid in contact with rheometer geometry)×10⁶. Engineering strain is the absolute value of compressive strain=ABS ((current compressed height of resilin solid (mm)−height of uncompressed solid (mm))/ht. of uncompressed solid (mm))*100.

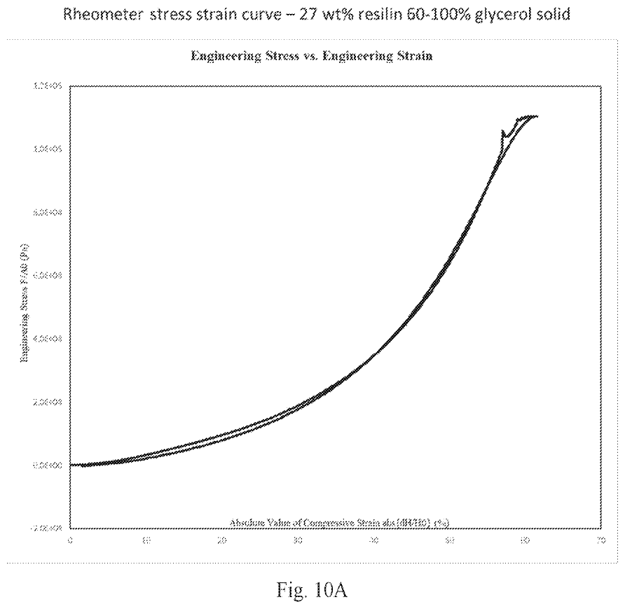

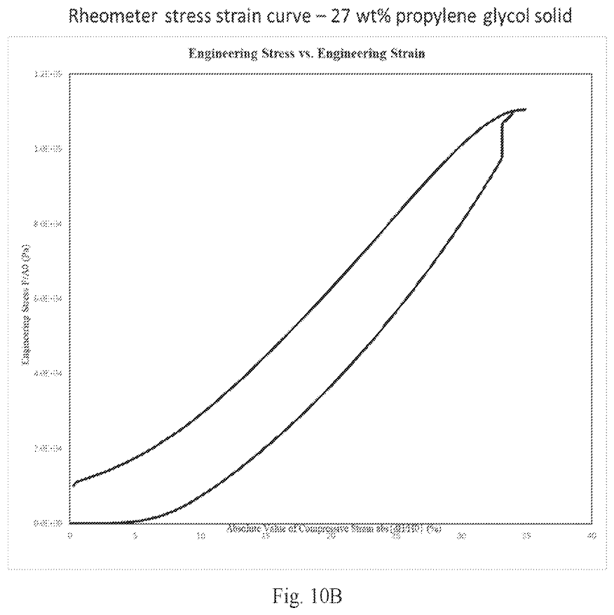

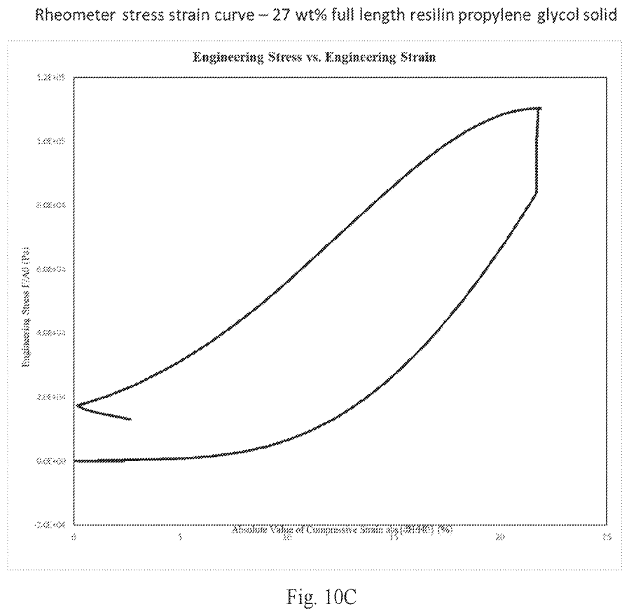

FIG. 10A shows a stress-strain curve for 27 wt % cross-linked resilin in 60-100% glycerol. FIG. 10B shows a stress-strain curve for 27 wt % cross-linked resilin in propylene glycol. FIG. 10C shows a stress-strain curve for 27 wt % full length cross-linked resilin in propylene glycol. For comparative purposes, rheometry data was also generated from Dr. Sholl's insert.

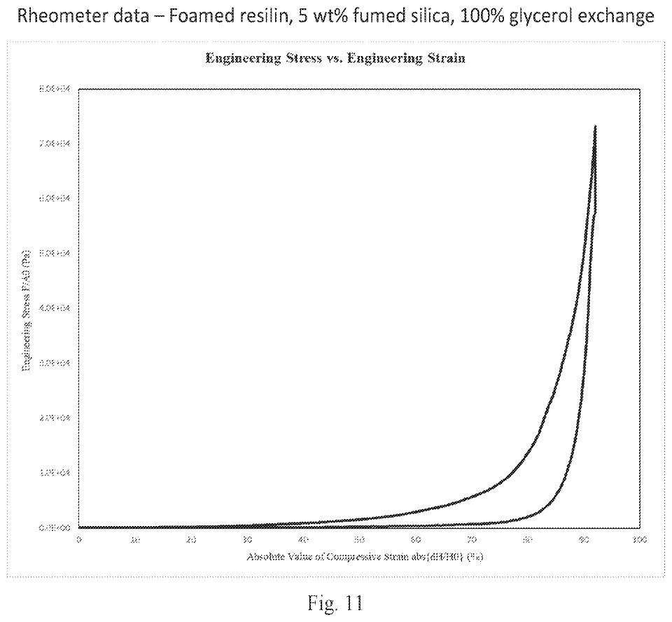

As shown in FIG. 11, the stress-strain curve of foamed resilin was shaped more like a hockey stick. This is a result of the air pockets in the foamed resilin, which are compressed first, followed by compression of the resilin material.

From the rheometry data, relative values of elastic modulus and resilience were determined as shown in Table 5. Relative Resilience (%) was calculated by taking the area under the load curve and dividing it by the area under the unload curve on the engineering stress vs strain plot and multiplying this value by 100. Relative Elastic modulus was calculated by taking the slope of the load curve from between 10 and 20% absolute compressive strain.

As shown, the exchange of cross-linked resilin solvent from an aqueous water-based solvent to a polar nonaqueous solvent resulted in a stiffer material with a similar resilience and a similar elasticity. All cross-linked resilin compositions were elastic under the conditions tested (<20 N Force).

A rheomoter was also used to measure resilience for a 5% by weight foamed resilin produced as described in Example 5 with a fumed silica as the blower in 100% glycerol solvent. The results are also shown in Table 6.

TABLE 6
Relative values of elastic modulus and resilience
for cross-linked resilin in different solvents

| | | | Elastic |
|---|---|---|---|
| | Sample | Resilience | Modulus |
| Solid Sample Description | ID | (%) | (kPa) |
| Dr Scholl's insert, 20 mm diameter | | 92 | 1.3 |
| 20 wt % resilin, propylene glycol | A | 59 | 4.9 |
| 27 wt % resilin, propylene glycol | B | 64 | 3.9 |
| 40 wt % resilin, propylene glycol | C | 59 | 6.6 |
| Resilin Coacervate, propylene glycol | D | 48 | 6.8 |
| 27 wt % full-length resilin, propylene | E | 34 | 8.5 |
| glycol | | | |
| 27 wt % resilin, 60-100% glycerol | F | 94 | 0.9 |
| 27 wt % resilin, 100% glycerol | H | 70 | 3.8 |
| 27 wt % resilin, ethylene glycol | I | 90 | 2.2 |
| 27 wt % resilin, water-based solid | J | 86 | 1 |
| 5 wt % foamed resilin (fumed silica), | | 35 | |
| 100% glycerol | | | |

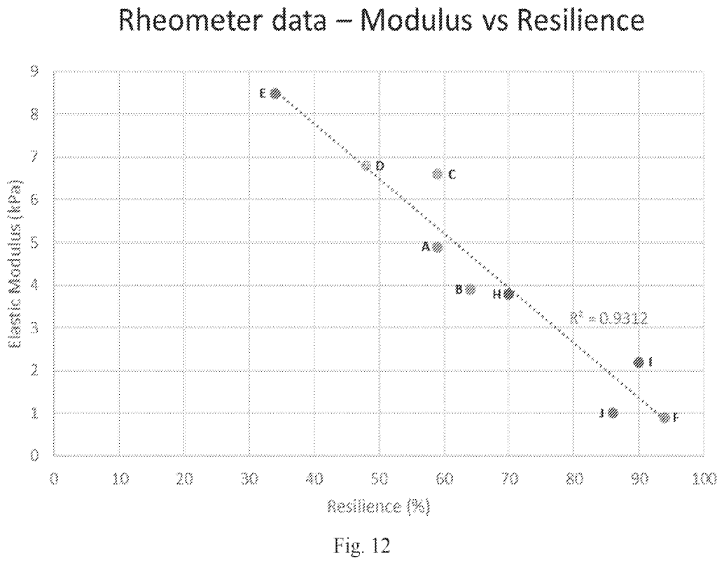

The relative values of resilience and elastic modulus from Table 6 were plotted as shown in FIG. 12. This plot shows the relationship between resilience and elastic modulus when tuning the cross-linked resilin properties via solvent exchange.

A rheometer was also used to measure relative resilience and elastic modulus for cross-linked resilin solids in propylene glycol prepared separately. The results are shown in Table 7.

TABLE 7
Rheometer data for identically prepared propylene glycol solids

| | Resilience | Elastic |
|---|---|---|
| Solid Sample Description | (%) | Modulus (KPa) |
| 27 wt % resilin, propylene glycol, solid 1 | 64 | 3.9 |
| 27 wt % resilin, propylene glycol, solid 2 | 59 | 5.8 |
| 27 wt % resilin, propylene glycol, solid 3 | 63 | 4.3 |

A rheometer was also used to measure relative resilience and elastic modulus for cross-linked resilin solids in different solvents through repeated applications of force to determine the effects of the repeated applications of force on these parameters. The results are shown in Table 8.

TABLE 8
Rheometer data: Resilience and modulus
data from multiple compressions

| | Resil- | Elastic |
|---|---|---|
| | ience | Modulus |
| Solid Sample Description | (%) | (KPa) |
| Dr. Scholl's insesrt, 20 mm diameter, 1^{st} compression | 92 | 1.3 |
| Dr. Scholl's insesrt, 20 mm diameter, 2^{nd} compression | 92 | 1.3 |
| Dr. Scholl's insesrt, 20 mm diameter, 3^{rd} compression | 95 | 1.3 |
| 27 wt % resilin, 60-100% glycerol, 1^{st} compression | 94 | 0.9 |
| 27 wt % resilin, 60-100% glycerol, 2^{nd} compression | 88 | 0.9 |
| 27 wt % resilin, 60-100% glycerol, 3^{rd} compression | 100 | 0.7 |
| 27 wt % resilin, propylene glycol, 1^{st} compression | 64 | 3.9 |
| 27 wt % resilin, propylene glycol, 2^{nd} compression | 64 | 3.8 |
| 27 wt % resilin, propylene glycol, 3^{rd} compression | 65 | 3.1 |
| 27 wt % resilin, propylene glycol, 4^{th} compression | 63 | 3.8 |
| 27 wt % resilin, propylene glycol, 5^{th} compression | 61 | 3.9 |
| 27 wt % full length resilin, propylene glycol, 1^{st} | 34 | 8.5 |
| compression | | |
| 27 wt % full length resilin, propylene glycol, 2^{nd} | 39 | 7.7 |
| compression | | |
| 27 wt % full length resilin, propylene glycol, 3^{rd} | 37 | 7.7 |
| compression | | |

### Example 8: Stiffness of Resilin Solids Vs. A Typical Midsole

Cross-linked silk solid compositions were prepared in water, 60-100% glycerol, 100% glycerol, propylene glycol (16 hour incubation), and propylene glycol (2 day incubation as described in Example 6. The stiffness of these materials was tested using a Shore OO Durometer (AD-100-00) via ASTM: D-2240. A Dr. Scholl's Insole and a typical midsole were also tested for comparative purposes. The results are shown in Table 9.

TABLE 9
Stiffness of different resilin compositions vs. a typical midsole

| Solid Sample Description | Hardness | Scale |
|---|---|---|
| Dr. Scholl's Insole | 35-45 | ◯◯ |
| Typical midsole | 55-63 | ◯◯ |
| Resilin - water-based | 0-4 | ◯◯ |
| Resilin - 60% to 100% glycerol | 10-16 | ◯◯ |
| Resilin - 100% glycerol | 25-35 | ◯◯ |
| Resilin - 100% propylene glycol | 30-40 | ◯◯ |
| Resilin - 100% propylene glycol - 2 d inc. | 50 | ◯◯ |
| Resilin foam, 5% wt fumed silica - 60% to | 10-30 | ◯◯ |
| 100% glycerol | | |

### Example 9: Elastic to Plastic Transition Measured by Zwick Compression Curves

Zwick compression data was obtained for 27 wt % resilin ethylene glycol solid (“A”), 27 wt % resilin propylene glycol solid (“C), coacervate resilin propylene glycol solid (“D”), 27 wt % resilin 60-100% glycerol solid (“G”), and 27 wt % resilin 100% glycerol solid (“H”). Each of these samples were prepared as described in Example 5.

Specifically, compression data for the above samples were tested using Zwick tensile tester with 5 kN load cell and 5 kN compression platens (Zwick/Roell Z5.0). Solid dimensions for each solid was a disc of 15 mm×5 mm. The materials were tested using a D695 Compressive Properties of Rigid Plastics compression program at a temperature of 22 C (ambient) and a humidity of 65% (ambient).

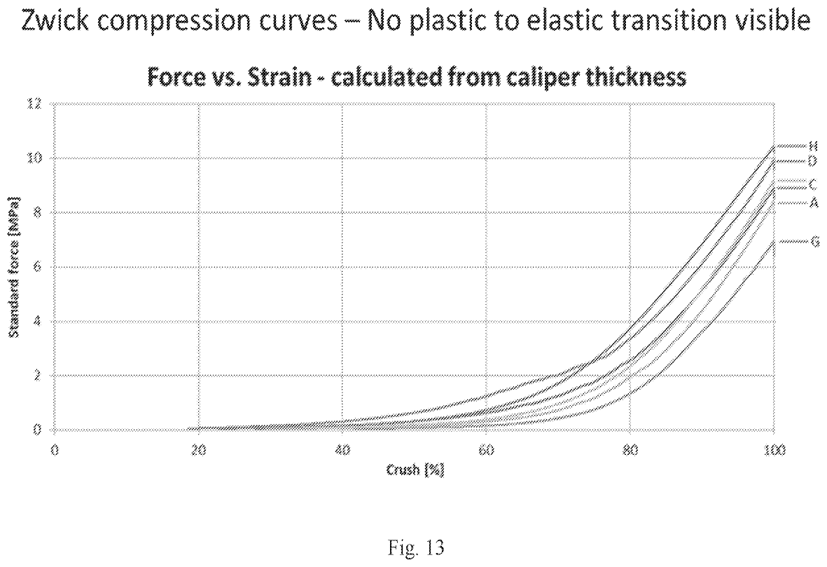

Force vs. strain curves as measured from the Zwick tensile tester for each solid are shown in FIG. 13. No elastic to plastic transition is visible for any of the resilin solids at up to 2 kN of applied compressive force. Instead, when the resilin solids broke under compressive stress it was via tearing and not elastic deformation.

### Example 10: Analysis of Resilience and Compression of Propylene Glycol Resilin Solids

Rebound resilience and compressive stress at 25% strain was measured using a ASTM D7121 and ASTM D575 test for 27 wt % resilin solid exchanged into propylene glycol, prepared as in Example 5. The dimensions of the solid tested were a 1.125 inch diameter disc with a height of 0.8 inches. 3 different samples with these dimensions were submitted. The results are shown in Table 10.

TABLE 10
Data on resilience and compression
of propylene glycol resilin solids

| Spec Ref | Test | Unit | A | B | C |
|---|---|---|---|---|---|
| ASTM D7121 | Rebound resilience | % | 46.2 | 39.2 | 52.3 |
| ASTM D575 | Compressive stress at 25% | psi | 6.7 | 8.1 | 6.2 |
| | strain | | | | |

The data shown in Table 10 can be compared to other rubbers or elastomers tested via the same ASTM test. The ASTM test number is sufficient to reproduce the results. This data and the durometer data are the only data that can be directly compared to other rubbers of elastomers.

### ADDITIONAL CONSIDERATIONS

The foregoing description of the embodiments of the disclosure has been presented for the purpose of illustration; it is not intended to be exhaustive or to limit the claims to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based hereon. Accordingly, the disclosure of the embodiments is intended to be illustrative, but not limiting, of the scope of the invention, which is set forth in the claims.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

SEQUENCE LISTING

| SEQ ID NO: | Species | Sequence |
|---|---|---|
| 1 | Drosophila | RPEPPVNSYLPPSDSYGAPGQSGAGGRPSDTYGAPGGGNGGRPSDSYGAPG |
| | sechellia | QGQGQGQGQGGYGGKPSDSYGAPGGGNGNGGRPSSSYGAPGGGNGGRPSDT |
| | | YGAPGGGNGGRPSDTYGAPGGGGNGNGGRPSSSYGAPGQGQGNGNGGRPSS |
| | | SYGAPGGGNGGRPSDTYGAPGGGNGGRPSDTYGAPGGGNNGGRPSSSYGAP |
| | | GGGNGGRPSDTYGAPGGGNGNGSGGRPSSSYGAPAQGQGGFGGRPSDSYGA |
| | | PGQNQKPSDSYGAPGSGNGSAGRPSSSYGAPGSGPGGRPSDSYGPPASGSG |
| | | AGGAGGSGPGGADYDNDEPAKYEFNYQVEDAPSGLSFGHSEMRDGDFTTGQ |
| | | YNVLLPDGRKQIVEYEADQQGYRPQIRYEGDANDGSGPSGPSGPGGPGGQN |
| | | LGADGYSSGRPGNGNGNGNGGYSSGRPGGQDLGPSGYSGGRPGGQDLGAGG |
| | | YSNVKPGGQDLGPGGYSGGRPGGQDLGRDGYSGGRPGGQDLGAGAYSNGRP |
| | | GGNGNGGSDGGRVIIGGRVIGGQDGGDQGYSGGRPGGQDLGRDGYSSGRPG |
| | | GRPGGNGQDSQDGQGYSSGRPGQGGRNGFGPGGQNGDNDGSGYRY |
| 2 | Drosophila | RPEPPVNSYLPPSDSYGAPGQSGAGGRPSDTYGAPGGGNGGRPSDSYGAPG |
| | sechellia | QGQGQGQGQGGYGGKPSDSYGAPGGGNGNGGRPSSSYGAPGGGNGGRPSDT |
| | | YGAPGGGNGGRPSDTYGAPGGGGNGNGGRPSSSYGAPGQGQGNGNGGRPSS |
| | | SYGAPGGGNGGRPSDTYGAPGGGNGGRPSDTYGAPGGGNNGGRPSSSYGAP |
| | | GGGNGGRPSDTYGAPGGGNGNGSGGRPSSSYGAPAQGQGGFGGRPSDSYGA |
| | | PGQNQKPSDSYGAPGSGNGSAGRPSSSYGAPGSGPGGRPSDSYGPPASG |
| 3 | Drosophila | GKPSDSYGAPGGGNGNGGRPSSSYGAPGGGNGGRPSDTYGAPGGGNGGRPS |
| | sechellia | DTYGAPGGGGNGNGGRPSSSYGAPGQGQGNGNGGRPSSSYGAPGGGNGGRP |
| | | SDTYGAPGGGNGGRPSDTYGAPGGGNNGGRPSSSYGAPGGGNGGRPSDTYG |
| | | APGGGNGNGSGGRPSSSYGAPAQGQGGFGGRPSDSYGAPGQNQKPSDSYGA |
| | | PGSGNGS |
| 4 | Acromyrmex | FGENRGNGGKPSTSYGVPDSNGNNRGGFGNGGSEGRPSTSYGLPDASRNNG |
| | echinatior | NGFGNVGNEDKPSTNYGIPANGNKVSGFGNVGSEGRPSTSYGVPGANGNQG |
| | | FGSGGIGGRPSTSYGVPGVNGNNGGGFENVGRPSTSYGTPDARGNNGGSFR |
| | | NGDIGGRPSTNYGIPGANGNHG |
| 5 | Aeshna | APSRGGGHGGGSISSSYGAPSKGSGGFGGGSISSSYGAPSKGSVGGGVSSS |
| | | YGAPAIGGGSFGGGSFGGGSFGGGSFGGGAPSSSYGAPSSSYSAPSSSYGA |
| | | PSKGSGGFGSSGGFSSFSSAPSSSYGAPSASYSTPSSSYGAPSSGGFGAGG |
| | | GFSSG |
| 6 | Aeshna | EPPVGGSQSYLPPSSSYGAPSAGTGFGHGGGSPSQSYGAPSFGGGSVGGGS |
| | | HFGGGSHSGGGGGGYPSQSYGAPSRPSGSSFQAFGGAPSSSYGAPSSQYGA |
| | | PSGGGGSYAIQGGSFSSGGSRAPSQAYGAPSNNAGLSHQSQSFGGGLSSSY |
| | | GAPSAGFGGQSHGGGYSQGGNGGGHGGSSGGGYSYQSFGGGNGGGHGGSRP |
| | | SSSYGAPSSSYGAPSGGKGVSGGFVSQPSGSYGAPSQSYGAPSRGGGHGGG |
| | | SISSSYGAPSKGSGGFGGGSISSSYGAPSKGSVGGGVSSSYGAPAIGGGSF |
| | | GGGSFGGGSFGGGSFGGGAPSSSYGAPSSSYSAPSSSYGAPSKGSGGFGSS |
| | | GGFSSFSSAPSSSYGAPSASYSTPSSSYGAPSSGGFGAGGGFSSGGYSGGG |
| | | GGYSSGGSGGFGGHGGSGGAGGYSGGGGYSGGGSGGGQKYDSNGGYVYS |
| 7 | Haematobia | AGGGNGGGGTGGTPSSSYGAPSNGGGSNGNGFGSPSSSYGAPGSGGSNGNG |
| | irritans | GGRPSLSYGAPGSGGSNGNGGGRPSSSYGAPGAGGSNGNGGGRPSSSYGAP |
| | | GAGGSNGNGGGRPSSSYGAPGAGGSNGNGGGRPSSSYGAPGAGGSNGNGGS |
| | | RPSSTYGAP |
| 8 | Haematobia | RPEPPVNSYLPPPLNNYGAPGAGGGSSDGSPLAPSDAYGAPDLGGGSGGSG |
| | irritans | QGPSSSYGAPGLGGGNGGAPSSSYGAPGLGGGNGGSRRPSSSYGAPGAGGG |
| | | NGGGGTGGTPSSSYGAPSNGGGSNGNGFGSPSSSYGAPGSGGSNGNGGGRP |
| | | SLSYGAPGSGGSNGNGGGRPSSSYGAPGAGGSNGNGGGRPSSSYGAPGAGG |
| | | SNGNGGGRPSSSYGAPGAGGSNGNGGGRPSSSYGAPGAGGSNGNGGSRPSS |
| | | TYGAPGAGGSNGNGCGNKPSSSYGAPSAGSNGNGGSEQGSSGSPSDSYGPP |
| | | ASGTGRGRNGGGGGAGGGRRGQPNQEYLPPNQGDNGNNGGSGGDDGYDYSQ |
| | | SGDGGGQGGSGGSGNGGDDGSNIVEYEAGQEGYRPQIRYEGEANEGGQGSG |
| | | GAGGSDGTDGYEYEQNGGDGGAGGSGGPGTGQDLGENGYSSGRPGGDNGGG |
| | | GGYSNGNGQGDGGQDLGSNGYSSGAPNGQNGGRRNGGGQNNNGQGYSSGRP |
| | | NGNGSGGRNGNGGRGNGGGYRNGNGNGGGNGNGSGSGSGNNGYNYDQQGSN |
| | | GFGAGGQNGENDGSGYRYS |
| 9 | Ctenocephalides | ANGNGFEGASNGLSATYGAPNGGGFGGNGNGGAPSSSYGAPGAGNGGNGGG |
| | felis | RPSSSYGAPGAGGSGNGFGGRPSSSYGAPGNGNGANGGRGGRPSSRYGAPG |
| | | NGNGNGNGNGGRPSSSYGAPGSNGNGGRPSSSYGAPGSGNGFGGNGGRPSS |
| | | SYGAPGANGNGNGGAIGQPSSSYGAPGQNGNGGGLSSTYGAPGAGNGGFGG |
| | | NGGGLSSTYGAPGSGNGGFGGNGLSSTYGAPGSGNGGFGGNGGGLSSTYGA |
| | | P |
| 10 | Ctenocephalides | PGGAGGAGGYPGGAGGAGGAGGYPGGSAGGAGGYPGGSGSGVGGYPGGSNG |
| | felis | GAGGYPGGSNGGAGGYPGGSNGGAGGYPGGSNGGAGGYPGGSNGNGGYSNG |
| | | GSNGGGAGGYPGGSNGNGGYPGSGSNGGAGGYPGGSNGNGGYPG |
| 11 | Bombus | FDGQNGIGGGDSGRNGLSNSYGVPGSNGGRNGNGRGNGFGGGQPSSSYGAP |
| | terrestris | SNGLGGNGGSGAGRPSSSYGAPGGGNGFGGGQPSSSYGAPSNGLGGNGAGR |
| | | PSSSYGAPGGGNGFGGGSNGAGKNGFGGAPSNSYGPPENGNGFGGGNGGGS |
| | | PSGLYGPPGRNGGNGGNGGNGGNGGRPSSSYGTPERNGGRPSGLYGPP |
| 12 | Tribolium | NGFGGGQNGGRLSSTYGPPGQGGNGFGGGQNGGRPSSTYGPPGQGGNGFGG |
| | castaneum | GQNGGRPSSTYGPPGQGGNGFGGGQNGGRPSSTYGPPGQGGNGFGGGQNGG |
| | | RPSSTYGPPGQGGNGFGGGQNGGRPSSTYGPPGQGGNGFGGGQNGGKPSST |
| | | YGPPGQGGNGFGGGQNGGRPSSTYGPPGQG |
| 13 | Tribolium | RAEPPVNSYLPPSQNGGPSSTYGPPGFQPGTPLGGGGNGGHPPSQGGNGGF |
| | castaneum | GGRHPDSDQRPGTSYLPPGQNGGAGRPGVTYGPPGQGGGQNGGGPSSTYGP |
| | | PGQGGNGFGGGQNGGRLSSTYGPPGQGGNGFGGGQNGGRPSSTYGPPGQGG |
| | | NGFGGGQNGGRPSSTYGPPGQGGNGFGGGQNGGRPSSTYGPPGQGGNGFGG |
| | | GQNGGRPSSTYGPPGQGGNGFGGGQNGGRPSSTYGPPGQGGNGFGGGQNGG |
| | | KPSSTYGPPGQGGNGFGGGQNGGRPSSTYGPPGQGGNGNGGGHNGQRPGGS |
| | | YLPPSQGGNGGYPSGGPGGYPSGGPGGNGGYGGEEESTEPAKYEFEYQVDD |
| | | DEHNTHFGHQESRDGDKATGEYNVLLPDGRKQVVQYEADSEGYKPKISYEG |
| | | GNGNGGYPSGGPGGAGNGGYPSGGPQGGNGGYPSGGPQGGNGGYPSGGPQG |
| | | GNGGYPSGGPQGGNGGYPSGGPQGGNGGYPSGGPQGGNGGYPSGGPQGGNG |
| | | GYPSGGPQGGNGGYTSGGPQGGNGGYPSGGPQGGNGGSGPY |
| 14 | Tribolium | QLTKRDAPLSGGYPSGGPANSYLPPGGASQPSGNYGAPSGGFGGKSGGFGG |
| | castaneum | SGGFGGAPSQSYGAPSGGFGGSSSFGKSGGFGGAPSQSYGAPSGGFGGSSS |
| | | FGKSSGGFGGAPSQSYGAPSGGFGGSSSFGKSGGFGGAPSQSYGAPSGGFG |
| | | GSSSFGKSGGFGGAPSQSYGAPSGGFGGKSSSFSSAPSQSYGAPSGGFGGK |
| | | SGGFGGAPSQSYGAPSGGFGGKSGGFGGAPSQSYGAPSGGFGGSSSFGKSG |
| | | GFGGAPSQSYGAPSGGFGGSSSFGKSSGFGHGSGAPSQSYGAPSRSQPQSN |
| | | YLPPSTSYGTPVSSAKSSGSFGGAPSQSYGAPSQSHAPSQSYGAPSRSFSQ |
| | | APSQSYGAPSQGHAPAPQQSYSAPSQSYGAPSGGFGGGHGGFGGQGQGFGG |
| | | GRSQPSQSYGAPAPSQSYGAPSAGGQQYASNGGYSY |
| 15 | Apis mellifera | RSEPPVNSYLPPSGNGNGGGGGGSSNVYGPPGFDGQNGIGEGDNGRNGISN |
| | | SYGVPTGGNGYNGDSSGNGRPGTNGGRNGNGNGRGNGYGGGQPSNSYGPPS |
| | | NGHGGNGAGRPSSSYGAPGGGNGFAGGSNGKNGFGGGPSSSYGPPENGNGF |
| | | NGGNGGPSGLYGPPGRNGGNGGNGGNGGRPSGSYGTPERNGGRLGGLYGAP |
| | | GRNGNNGGNGYPSGGLNGGNGGYPSGGPGNGGANGGYPSGGSNGDNGGYPS |
| | | GGPNGNGNGNGGYGQDENNEPAKYEFSYEVKDEQSGADYGHTESRDGDRAQ |
| | | GEFNVLLPDGRKQIVEYEADQDGFKPQIRYEGEANSQGYGSGGPGGNGGDN |
| | | GYPSGGPGGNGYSSGRPNGGSDFSDGGYPSTRPGGENGGYRNGNNGGNGNG |
| | | GYPSGNGGDAAANGGYQY |
| 16 | Apis mellifera | DAPISGSYLPPSTSYGTPNLGGGGPSSTYGAPSGGGGGRPSSSYGAPSSTY |
| | | GAPSSTYGAPSNGGGRPSSTYGAPSNGGGRPSSSYGAPSSSYGAPSSTYGA |
| | | PSNGGGRPSSSYGAPSFGGGGGFGGGNGLSTSYGAPSRGGGGGGGSISSSY |
| | | GAPTGGGGGGPSTTYGAPNGGGNGYSRPSSTYGTPSTGGGSFGGSGGYSGG |
| | | GGGYSGGGNGYSGGGGGGYSGGNGGGYSGGGNGGGYSGGNGGGYSGGGGGG |
| | | YSGGGGGGYSGGGNGYSGGGGGGYSGGNGGYSGGNGGYSGGGGGYSGGGGG |
| | | GQSYASNGGYQY |
| 17 | Nasonia | RPEPPVNSYLPPGQGGGFGGGRPSGASPSDQYGPPDFQGAGGRGGQAAGGN |
| | vitripennis | FGGGGNGFGGAPSSSYGPPGFGSNEPNKFSGAGGGGAGRPQDSYGPPAGGN |
| | | GFAGSAGAGNSGRPGGAAAGGRPSDSYGPPQGGGSGFGGGNAGRPSDSYGP |
| | | PSAGGGGFGGGSPGGGFGGGSPGGGFGGGNQGAPQSSYGPPASGFGGQGGA |
| | | GQGRPSDSYGPPGGGSGGRPSQGGNGFGGGNAGRPSDSYGPPAAGGGGFGG |
| | | NAGGNGGGNGFGGGRPSGSPGGFGGQGGGGRPSDSYLPPSGGSGFGGGNGR |
| | | QPGGFGQQGGNGAGQQNGGGGAGRPSSSYGPPSNGNGGGFSGQNGGRGSPS |
| | | SGGGFGGAGGSPSSSYGPPAGGSGFGNNGGAGGRPSSSYGPPSSGGNGFGS |
| | | GGQGGQGGQGGQGGRPSSSYGPPSNGNGGFGGGNGGRPSSNGYPQGQGNGN |
| | | GGFGGQGGNGGRPSSSYGPPGGDSGYPSGGPSGNFGGSNAGGGGGGFGGQV |
| | | QDSYGPPPSGAVNGNGNGYSSGGPGGNGLDEGNDEPAKYEFSYEVKDDQSD |
| | | GRKQIVEYEADQDGFKPQIRYEGEANTGAGGAGGYPSGGGGDSGYPSGPSG |
| | | AGGNAGYPSGGGGGAGGFGGNGGGSNGYPSGGPSGGQGQFGGQQGGNGGYP |
| | | SGPQGGSGFGGGSQGSGSGGYPSGGPGGNGGNNNFGGGNAGYPSGGPSGGN |
| | | GFNQGGQNQGGSGGGYPSGSGGDAAANGGYQYS |
| 18 | Nasonia | RAEAPISGNYLPPSTSYGTPNLGGGGGGGGGFGGGAPSSSYGAPSSGGGFG |
| | vitripennis | GSFGGGAPSSSYGAPSTGGSFGGGAPSSSYGAPSSGGSFGGSFGGGAPSSS |
| | | YGAPSFGGNAPSSSYGAPSAGGSFGGGAPSNSYGPPSSSYGAPSAGGSFGG |
| | | SSGGSFGGSFGGGAPSSSYGAPAPSRPSSNYGAPSRPSSNYGAPSSGGSGF |
| | | GGGSGFGGGRPSSSYGAPSSGSFGGGFGGGAPSSSYGAPAPSRPSSNYGAP |
| | | APSRPSSNYGAPAPSRPSSSYGAPSRPSSNYGAPSRPSSNYGAPSSGGSGF |
| | | GGGSGFGGGRPSSSYGAPSSGSFGGGFGGGAPSSSYGAPAPSRPSSNYGPP |
| | | SSSYGAPSSGGSGGFGGGAPSSSYGAPSFGGSSNAVSRPSSSYGAPSSGGG |
| | | QSYASNGGYQY |
| 19 | Pediculus | EPPVKTSYLPPSASRSLNSQYGAPAFTDSNELVAPSPNSNFHDSYNQQQQS |
| | humanus | FDLSNGLSVPSAAGRLSNTYGVPSAQGANVPSFDSSDSIAVDAAGRSGNSF |
| | corporis | SSHVPSSTYGAPGNGFGGGSRSSQSGAPSSVYGPPQARNNNFGNGAAPSSV |
| | | YGPPQARNNNFGNGGAPSQVYGPPKARNNNFGNGAAPSSVYGPPQARNNNF |
| | | GNGAAPSSVYGPPQARNNNFANSAAPSQVYGPPQARNNNFGNGAAPSSVYG |
| | | PPQSSSFSSPSGRSGQLPSATYGAPFERNGFGSQGSSGFQGYEPSKRSQTT |
| | | EDPFAEPAKYEYDYKVQASDETGTEFGHKESRENESARGAYHVLLPDGRMQ |
| | | IVQYEADETGYRPQIRYEDTGYPSAASSRSNNGFNGYQY |
| 20 | Anopheles | KREAPLPPSGSYLPPSGGAGGYPAAQTPSSSYGAPTGGAGSWGGNGGNGGR |
| | gambiae str. | GHSNGGGSSFGGSAPSAPSQSYGAPSFGGQSSGGFGGHSSGGFGGHSSGGH |
| | PEST | GGNGNGNGNGYSSGRPSSQYGPPQQQQQQQSFRPPSTSYGVPAAPSQSYGA |
| | | PAQQHSNGGNGGYSSGRPSTQYGAPAQSNGNGFGNGRPSSSYGAPARPSTQ |
| | | YGAPSAGNGNGYAGNGNGRSYSNGNGNGHGNGHSNGNGNNGYSRGPARQPS |
| | | QQYGPPAQAPSSQYGAPAQTPSSQYGAPAQTPSSQYGAPAQTPSSQYGAPA |
| | | QTPSSQYGAPAPSRPSQQYGAPAPSRPSQQYGAPAQTPSSQYGAPAQTPSS |
| | | QYGAPAQTPSSQYGAPAQTPSSQYGAPAQQPSSQYGAPAPSRPSQQYGAPA |
| | | QQPSAQYGAPAQTPSSQYGAPAPSRPSQQYGAPAQAPSSQYGAPAPSSQYG |
| | | APAQQPSSQYGAPAQTPSSQYGAPSFGPTGGASFSSGNGNVGGSYQVSSTG |
| | | NGFSQASFSASSFSPNGRTSLSAGGFSSGAPSAQSAGGYSSGGPSQVPATL |
| | | PQSYSSNGGYNY |
| 21 | Glossina | RPEPPVNTYLPPSAGGGSGGGSPLAPSDTYGAPGVNGGGGGGGGPSSTYGA |
| | morsitans | PGSGGGNGNGGGGFGKPSSTYGAPGLGGGGNGGGRPSETYGAPSGGGGNGF |
| | | GKPSSTYGAPNGGGGNGGPGRPSSTYGAPGSGGGNGGSGRPSSTYGAPGLG |
| | | GGNGGSGRPSSMYGAPGLGGGNGGSGRPSSTYGAPGSGGGNGGSGRPSSTY |
| | | GAPGSGGGNGGSGRPSSTYGAPGNGNGGNGFGRPSSTYGAPGSGGSNGNGK |
| | | PSSTYGAPGSGGGGGRPSDSYGPPASGNGGRNGNGNGQSQEYLPPGQSGSG |
| | | GGGGYGGGSGSGGSGGGGGGGYGGDQDNNVVEYEADQEGYRPQIRYEGDGS |
| | | QGGFGGDGDGYSYEQNGVGGDGGGAGGAGGYSNGQNLGANGYSSGRPNGGN |
| | | GGGRRGGGGGGGGSGGGQNLGSNGYSSGAPNGFGGGNGQGYSGGRSNGNGG |
| | | GGGGRNGGRYRNGGGGGGGRNGGGSNGYNYDQPGSNGFGRGGGNGENDGSG |
| | | YHY |
| 22 | Atta | RSEPPVNSYLHPGSDTSGTNGGRTDLSTQYGAPDFNNRGNGNSGATSFGGS |
| | cephalotes | GAGNGPSKLYDVPIRGNTGGNGLGQFRGNGFESGQPSSSYGAPKGGFGENR |
| | | GNRGRPSTSYGVPDSNRNNRGGFGNGGSEARPSTSYGVPGANGNQGGFGSG |
| | | SIGGRPSTSYGVPGANGNNGDSFRNGDIGGRPSTNYGAPGANGNHGGGNGG |
| | | NGRPSNNYGVPGANGNTNGKGRLNGNSGGGPSNNYGSPNGFGKGLSTSYGS |
| | | PNRGGNDNHYPSRGSFINGGINGYSSGSPNGNAGNFGHGDESFGRGGGEGE |
| | | NTGEGYNANAQEESTEPAKYEFSYKVKDQQTGSDYSHTETRDGDHAQGEFN |
| | | VLLPDGRKQIVEYEADQDGFKPQIRYEGEANADGGYGSGLNDNNDGYSSGR |
| | | PDSESGGFANSGFNGGSSNGGYPNGGPGERKLGGFNNGGSSGYQSGRSAGQ |
| | | SFGRDNAGDLNNDIGGYFSNSPNNIGDSDNANVGSNRQNDGNSGYQY |
| 23 | Anopheles | KREAPLPPSGSYLPPSGGGGGGGGYPAAQTPSSSYGAPAGGAGGWGGNGNG |
| | darlingi | NGNGNGGRGGYSNGGGHSGSAPSQSYGAPSAPSQSYGAPSQSYGAPAAAPS |
| | | QSYGAPSFGGNGGGASHGSGGFTGGHGGNGNGNGYSSGRPSSQYGPPQQQQ |
| | | QPQQQSFRPPSTSYGVPAAPSSSYGAPSANGFSNGGRPSSQYGAPAPQSNG |
| | | NEFGAPRPSSSYGAPSRPSTQYGAPSNGNGNGYAGHGNGNGHGNGNGHSNG |
| | | NGNGYNRGPARQPSSQYGPPSQGPPSSQYGPPSQYGPPSSGTSFIAYGPPS |
| | | QGPPSSQYGAPAPSRPSSQYGAPAQTPSSQYGAPAQTPSSQYGPPRQSSPQ |
| | | FGAPAPRPPSSQYGAPAQAPSSQYGAPAQTPSSQYGAPAQAPSSQYGAPAP |
| | | SRPSSQYGVPAQAPSSQYGAPAQAPSSQYGAPAQTPSSQYGAPSFGSTGGS |
| | | SFGGNGGVGGSYQTASSGNGFSQASFSASSFSSNGRSSQSAGGYSSGGPSQ |
| | | VPATIPQQYSSGGGSYSSGGHSQVPATLPQQYSSNGGYNY |
| 24 | Acromyrmex | RSEPPVNSYLPPGPGTSGANGGQTDLSIQYRASDFNNRGNVNGNSGATSFG |
| | echinatior | GPGASNGPSKLYDVPIGGNAGGNGLGQFRGNGFEGGQPSSSYGAPNGGFGE |
| | | NRGNGGKPSTSYGVPDSNGNNRGGFGNGGSEGRPSTSYGLPDASRNNGNGF |
| | | GNVGNEDKPSTNYGIPANGNKVSGFGNVGSEGRPSTSYGVPGANGNQGFGS |
| | | GGIGGRPSTSYGVPGVNGNNGGGFENVGRPSTSYGTPDARGNNGGSFRNGD |
| | | IGGRPSTNYGIPGANGNHGGGNGGNGRPSSNYGVPGGNGNTNGKGRFNGNS |
| | | GGRPSNSYGSPNGFGKGLSTSYSPSNRDGNGNHYPSGDSNRGSFVNGGING |
| | | YPSGSPNGNAGNFRHGDESFGRGGEGGGRSTGEGYNANAQEESTEPAKYEF |
| | | SYKVKDQQTGSDYSHTETRDGDHAQGEFNVLLPDGRKQIVEYEADQDGFKP |
| | | QIRYEGEANADGEYDSGGLNDNNDGYSSGRPGSESGGFANNSGFNGGSSNG |
| | | GYPSGGSGEGKLGFNSGGNSGYQSGRPAGQSFGRDNAGDLSNDIGGFSNSP |
| | | NNIGGDNANVGSNRQNGGNSGYQY |
| 25 | Acyrthosiphon | ESPYGGGSSNSNGNGRNGGYGGKGQYGGGNGGGVGSSSASPFFSGANQYGS |
| | pisum | QSGLSGAANNRYPSFGSKFGGNKGSYGGSSSRNNGRYGSGSASGYGSGSSG |
| | | GLGSTGRSTGGYGGGSSGSYGSGSSGSLGSSTGSNGIYGAGSSGGFGSGSS |
| | | GSYGGGSSGGFGSGSSGSYGGGSSGGFGSGSSGSYGGGSSGGFGSGSSGSY |
| | | GGGSSGGFGSGSSGNYGSGSSGSYGSGGGGLGGASSGNNDGYGAGGSGSYD |
| | | QLGGANGNGLGGSGNDPLSEPANYEFSYEVNAPESGAIFGHKESRQGEEAT |
| | | GVYHVLLPDGRTQIVEYEADEDGYKPKITYTDPVGGYAGDRQSGNSYGGNG |
| | | GFGGSGSLGGSGGNLGGLYNGGGSSNNGAGYGGSSSSLGSRYGGSGGSSGS |
| | | GVGGGYGGSGSSSGGIGSSYGGSGSLSGGLGGGYGGSGSSSGGLGGGYGGS |
| | | GGSSGGGFGGLGGSGGSSGSGYGGSGSSSGGLGNSYGGSGSSNGGLGGGYS |
| | | GSGGSSGGLGGGYGASSGSSGSGLGGGYGGSGSSSGGLGSGYGGLGSSSGG |
| | | LGGGYGGSGSSSGGLGGGYGGSGSSNGGIGGGYGGSSGSSGGLGGGYGGSG |
| | | SSSGGLGGGYGGSGGSNSGLGSSYGGSGSTNGGLGGGYGGLGSSSGGLGGG |
| | | YGGSGGSNGGIGGGYGGSSGSGGSQGSAYGGSGSSSGSQGGGYGGSGSSSG |
| | | GLGGGYGSSSGSSSGLGGSYGSNRNGLGSGSSYS |
| 26 | Drosophila | RPEPPVNSYLPPSPGDSYGAPGQGQGQGQGGFGGKPSDSYGAPGAGNGNGN |
| | virilis | GRPSSSYGAPGQGQGQGGFGGKPSDSYGAPGAGNGNGNGRPSSSYGAPGQG |
| | | QGQGGFGGRPSSSYGAPGQGQGGFGGKPSDTYGAPGAGNGNGRPSSSYGAP |
| | | GQGQGGIGGKPSDSYGAPGAGNGNGNGRPSSSYGAPGQGQGGFGGKPSDTY |
| | | GAPGAGNGNGRPSSSYGAPGQGQGGFGGKPSDTYGAPGAGNGNGNGRPSSS |
| | | YGAPGQGQGGFGGKPSDTYGAPGAGNGNGRPSSSYGAPGQGQGQGGFGGKP |
| | | SDSYGPPASGAGAGGAGGPGAGGGGDYDNDEPAKYEFNYQVEDAPSGLSFG |
| | | HSEMRDGDFTTGQYNVLLPDGRKQIVEYEADQQGYRPQVRYEGDANGNGGP |
| | | GGAGGPGGQDLGQNGYSSGRPGGQDLGQGGYSNGRPGGQDLGQNGYSGGRP |
| | | GGQDLGQNGYSGGRPGGQDLGQNGYSGGRPGGQDLGQNGYSGGRPGGQDLG |
| | | QNGYSGGRPGGQDLGQNGYSGGRPGGNGGSDGGRVIIGGRVIGQDGGDGQG |
| | | YSSGRPNGQDGGFGQDNTDGRGYSSGKPGQGRNGNGNSFGPGGQNGDNDGS |
| | | GYRY |
| 27 | Drosophila | RPEPPVNSYLPPSDSYGAPGQSGPGGRPSDSYGAPGGGNGGRPSDSYGAPG |
| | erecta | LGQGQGQGQGQGGFGGKPSDSYGAPGAGNGNGGRPSSSYGAPGAGNGGRPS |
| | | DTYGAPGGGSGGRPSDTYGAPGGGNGNGNGGRPSSSYGAPGQGQGNGNSGR |
| | | PSSSYGAPGAGNGGRPSDTYGAPGGGNGGRPSSSYGAPGAGNGGNGGRPSD |
| | | TYGAPGGGNGNGNGNGNGSGGRPSSSYGAPGQGQGGFGGRPSDSYGAPGQN |
| | | QKPSDSYGAPGSGSGSGNGNGGRPSSSYGAPGSGPGGRPSDSYGPPASGSG |
| | | AGGAGGSGPGGADYDNDIVEYEADQQGYRPQIRYEGDANDGSGPSGPGGQN |
| | | LGADGYSSGRPGNGNGNGNGGYSGGRPGGQDLGPSGYSGGRPGGQDLGAGG |
| | | YSNGRPGGQDLGPSGYSGGRPGGQDLGPSGYSGGRPGGQDLGAGGYSNGRP |
| | | GGNGNGNGGADGGRVIIGGRVIGGQDGGDQGYSGGRPGGQDLGRDGYSSGR |
| | | PGGRPGANGQDNQDGQGYSSGRSGKGGRNSFGPGGQNGDNDGSGYRY |
| 28 | Lutzomyia | RPEPPANTYLPPSSSYAAPGQQGGSGFGGGGGSGGSGGFGQPGAFGRPSSS |
| | longipalpis | YGPPSQGGAGGGFGSDSQFGGGFGGGAGGFGSGGSGAPGASQRPSSSYGPP |
| | | GQTGGGGFGAQGAPGSSFGPGGGFGGGSPGQAGSPGFQRPSSSYGPPGQSP |
| | | GGGFSQQGGAPGASQRPSSTYGAPGQGAGGFGQGGSGGFGGTGGSVAIGGR |
| | | PSSSYGAPGQGSSGGFGGGSGGFGSQAPSTSYGAPGQGSPGGGFGSQGGPG |
| | | GQPGSPGFGGSQRPSSSYGPPGQGGAPGQGGSPGFGASSRSGGAGGFGASQ |
| | | QPSSSYGPPGQGAGSGFQGTGGGFGGPGQRPGFGGSQTPATSYGAPGQAGG |
| | | ASGGFGGAGAQRPSSSYGPPGQASGFGGGSSGGGFGGGSSGGFGGNQGGFG |
| | | GNQGGFGGSQTPSSSYGAPSFGSGGSPGAAGGAGGFGQGGVGGSGQPGGFG |
| | | GGDQGYPPRGGPGGFGPGSGGSGAGGPIAGGSGSGYPGGSDSGSNEPAKYD |
| | | FSYQVDDPASGTSFGHSEQRDGDYTSGQYNVLLPDGRKQIVEYEADLGGYR |
| | | PQIKYEGGSSGGAGGYPSGGPGSQGGAGGYPSGGPGGPGSPGGAGGYQSGA |
| | | AGGAGGYPSGGPGGPGAGGYPSGGPGGPGSQAGGFSGGFGGGSDGAFGGAG |
| | | GFSQGGAGGGDAGYPRGGPGGFGGAGSPGFGGSGSPGFGGSGSPGAQGSSG |
| | | FGGTGGGFGGGADGYPRGGPGAGQSGFQDGRGATGGAGQPGGRGSFGRPGS |
| | | ARGGSSSNGYANGGAEGYPRDNPQNRGSGYS |
| 29 | Rhodnius | KRDDPLRRFLAPLVGGGNGSGGGGGGYNYNKPANGLSLPGGGGALPPATSY |
| | prolixus | GVPDRPAPVPSSPPSSSYGAPQPSPNYGAPSSSYGAPSQQPSRSYGAPSQG |
| | | PSTSYSQRPSSSYGAPAPQTPSSSYGAPAQQPSGSYGAPSGGGGSSGYTGG |
| | | AQRPSGSYGAPSQGGPSGNYGPPSQQPSSNYGAPSQTPSSNYGAPAQRPST |
| | | SYGAPSQPPSSSYGSPPQRASGYPSSSSGPSNGYSPPAQRPSSSYGPPSQQ |
| | | PASSYGAPSQTPSSNYGPPAPIPSSNYGAPSQPPSKPSAPSSSYGTPSQTP |
| | | STSYGAPSQAPSSSYGAPSRPSPPSSSYGAPSQGPSSSYGPPSRPSQPSSP |
| | | SSGYGAPSQGPSSSYGAPSRPSSPSSSYGAPPSSSYGAPSRPSPPSSSYGA |
| | | PSQGPSSSYGPPSRPSQPSSPSSGYGAPSQGPSSSYGAPSRPSSPSSSYGA |
| | | PPSSSYGAPSRPSPPSSSYGAPSQGPSSSYGPPSRPSQPSSPSSSYGAPSQ |
| | | GPSSSYGAPSRPSPPSSSYGAPSQGPSSSYGPPSRPSQPSSTYGVPSGGRP |
| | | STPSSSYGAPPQALSSTYGAPSGRPGAPSQKPSSSYGAPSLGGNASRGPKS |
| | | SPPSSSYGAPSVGTSVSSYAPSQGGAGGFQSSRPSSSYGAPSTGPSSTYGP |
| | | PSQPPSSSYGVPSQPPSSNYGVPSQGVSGSVGSSSPSSSYGAPSQIPSSSY |
| | | GAPSQSSIGGFGSSRPSSSYGAPPQAPSSSYSAPLRAPSTSYGAPSGGSGS |
| | | NFGSKPSTNYGAPSQPPSTNYGPPSQPPSSSYGTPSRAPSPTYSTPQSSGT |
| | | SFGSRPSSSYGVPSQPTTNYGAPSQTPSSNYGAPPASSAPSSTYGRPSQSP |
| | | SSSYGAPSPSSSSSSYESPSQPPSSSYGAPSQGPSSSYGAPSRPSSTYGAP |
| | | SPSSPSTNYGAPAPSSNYGTPAQDLTGSYAAPSQPPSAGYGAPSGQPSSGG |
| | | KQNFQVKNPFAGQTHQVYPAVSSISFGLPSQSFNTAIQGQEPSQSYGAPTA |
| | | SSPSSSYGAPTGTGSSQPGQSYASNGGYSYS |
| 30 | Rhodnius | QPPFNHYLPAARGSGSNSAQYTAPSSKFGTSTGQYGQPPSEVPRGLQQGSY |
| | prolixus | AEDVHSSRSVNPSSQNGIPSGHFSSLSSNYGAPSSDYSRSFLRYGTLSNKY |
| | | GVPNSALGSLSSRNNKTPATQLSYQPSSHYDSRSTSEDQFISSRVSDSQYG |
| | | ASSVRRFLPSSQYSTPSSQYGTPSSQYGTPSSQYGTPSSQYGTPSSQYGTP |
| | | SSQYGTPSSQYGTPSSQYGTPSSQYGTPSSQYGTPSSPPSQYGGPYSMRTS |
| | | APNSQYGTPSSFRTSPSSQFGSSSAHSSSLSKFRSVPSSPYGTLSAIRSTH |
| | | SSQYGTPSSFSDSTSSSHNGLPSHYPGSGFSGSSVNDQKSYTGNVFGQSHS |
| | | RVANGDQHARSYTLAGGNEISEPAKYDFNYDVSDGEQGVEFGQEESRDGEE |
| | | TNGSYHVLLPDGRRQRVQYTAGQYGYKPTISYENTGTLTTGRQQFSNGFYN |
| | | VQQSGSESQEHLGRSTGQNSYGGSNGYESGVGYQSGVGRRSRPAGSY |
| 31 | Solenopsis | RSEPPINSYLPPRAGSSGANGGRTDLTTQYGAPDFNNGGGATSFSGNGAGD |
| | invicta | GPSKLYDVPVRGNAGGNGLGRGNGFGGGQPSSSYGAPNGGSNENRGNGGRP |
| | | STSYGVPGANGNNGGGFGNGGDKGRPSTSYGVPDASGSSQGSFGNVGNGGR |
| | | PSTNYGVPGANGNGGGFGNAANEGKPSTSYGVPGANGNSQGGFGNGGRPST |
| | | GYGVPGANGNNGGGFGGRPSTSYGAPGANGNHRGGNGGNASPSTNYGVPGG |
| | | NNGNTNGKGRFNGGNSGGGPSNNYGVPNENAFGGGLSTSYGPPSRGGNGNS |
| | | GYPSGGSNGGSFVNNGANGYPSGGPNGNAGNFGDGRGGKGGGSSGEGYNDN |
| | | AQEGSTEPAKYEFSYKVKDQQTGSEYSHTETRDGDRAQGEFNVLLPDGRKQ |
| | | IVEYEADQDGFKPQIRYEGEANAGGGYSSGGSNDNNDGYSSGRPGSEAGGF |
| | | ANNSGFNGSGTNGGRSSGGPGDGNPGGFNSGGGGGYQSGRPAGQSFGRDND |
| | | GGLSGDIGGYFANSPSNNIGGSDSANVGSNRQNGGNGGYQY |
| 32 | Culex | KREAPLPGGSYLPPSNGGGAGGYPAAGPPSGSYGPPSNGNGNGNGAGGYPS |
| | quinquefasciatus | APSQQYGAPAGGAPSQQYGAPSNGNGGAGGYPSAPSQQYGAPNGNGNGGFG |
| | | GRPQAPSQQYGAPSNGNGGARPSQQYGAPNGGNGNGRPQTPSSQYGAPSGG |
| | | APSSQYGAPSGGAPSQQYGAPNGGNGNGRPQTPSSQYGAPSGGAPSQQYGA |
| | | PNGGNGNGRPQTPSSQYGAPSGGAPSSQYGAPSGGAPSSQYGAPAGGAPSS |
| | | QYGAPAGGAPSSQYGAPAGGAPSSQYGAPAGGAPSSQYGAPAGGAPSSQYG |
| | | APSSQYGAPAGGAPSSQYGAPAGGAPSSQYGAPSGGAPSSQYGAPSGGAPS |
| | | SQYGAPAGGAPSSQYGAPSGGAPSS |
| 33 | Bactrocera | RPEPPVNSYLPPSANGNGNGGGRPSSQYGAPGLGSNSNGNGNGNGGGRPSS |
| | cucurbitae | QYGVPGLGGNGNGNGNGGGGGRPSSSYGAPGLGGNGNGNGNGGGRPSSQYG |
| | | VPGLGGNGNGNGNGNGGGRPSSTYGAPGLRGNGNGNGNGNGRPSSTYGAPG |
| | | LGGNGNGNGNGNGRPSSTYGAPGLGGNGNGNGNGNGRPSSTYGAPGLNGNG |
| | | LGGGQKPSDSYGPPASGNGNGYSNGGNGNGNGGGRPGQEYLPPGRNGNGNG |
| | | NGGRGNGNGGGANGYDYSQGGSDSGESGIVDYEADQGGYRPQIRYEGEANN |
| | | GAGGLGGGAGGANGYDYEQNGNGLGGGNGYSNGQDLGSNGYSSGRPNGNGN |
| | | GNGNGNGNGYSGRNGKGRNGNGGGQGLGRNGYSDGRPSGQDLGDNGYASGR |
| | | PGGNGNGNGGNGNGYSNGNGYSNGNGNGTGNGGGQYNGNGNGYSDGRPGGQ |
| | | DNLDGQGYSSGRPNGFGPGGQNGDNDGNGYRY |
| 34 | Trichogramma | RPEPPVNSYLPPGQGGQGGFGGSGGRPGGGSPSNQYGPPNFQNGGGQNGGS |
| | pretiosum | GFGGNGNGNSFGPPSNSYGPPEFGSPGAGSFGGGRPQDTYGPPSNGNGNGN |
| | | GFGGNGNGGGRPSSRPSDSYGPPSSGNGFGGGNSGRPSESYGPPQNGGGSG |
| | | NGNQGGGNGFGNGGGRGGQGKPSDSYGPPNSGNRPGSSNGGGQQQNGFGGG |
| | | NGGRPSNTYGPPGGGNGGGRPGGSSGGFGGQNGGRPSDSYGPPSNGNGNGG |
| | | RPSNNYGPPNSGGGNGNGFGGSNGKPSNSYGPPSNGNGGGFGGSNGRPSNS |
| | | YGPPSGGNGGGFGGSSAVGRPGNSGSPSSSGSGFGGNGGASRPSSSYGPPS |
| | | NGGGFGNGGGSNGRPSSSYGPPNSGSNGGGFGGQNGNGRQNGNNGQGGFGG |
| | | QPSSSYGPPSNGNGFGGGGGSNGYPQNSQGGNGNGFGQGSGGRPSSSYGPP |
| | | SNGGGGGDNGYSSGGPGGFGGQPQDSYGPPPSGAVDGNNGFSSGGSSGDNN |
| | | GYSSGGPGGNGFEDGNDEPAKYEFSYEVKDEQSGSSFGHTEMRDGDRAQGE |
| | | FNVLLPDGRKQIVEYEADQDGFKPQIRYEGEANTGGAGGYPSGGPGGQGGN |
| | | GNGGYPSGGPSNGGFGGQNGGGNGGYPSGGPSGGGFGGQNGGSGGYPSGGP |
| | | SGGGFGGQGGFGGQNSGGNGGYSSGGPASGGFGGQNGGNGGYPSGGPSGGG |
| | | FGGQGGFGGQNSGGNGGYPSGGPSSGGFGGQNGGGGGNYPAGSGGDAEANG |
| | | GYQYS |
| 35 | Drosophila | QSGAGGRPSDTYGAPGGGNGGRPSDSYGAPGQGQGQGQGQGGYGGKPSDSY |
| | sechellia | GAPGGGNGNGGRPSSSYGAPGGGNGGRPSDTYGAPGGGNGGRPSDTYGAPG |
| | | GGGNGNGGRPSSSYGAPGQGQGNGNGGRPSSSYGAPGGGNGGRPSDTYGAP |
| | | GGGNGGRPSDTYGAPGGGNNGGRPSSSYGAPGGGNGGRPSDTYGAPGGGNG |
| | | NGSGGRPSSSYGAPAQGQGGFGGRPSDSYGAPGQNQKPSDSYGAPGSGNGS |
| | | AGRPSSSYGAPGSGPGGRPSDSYGP |
| 36 | Drosophila | QSGAGGRPSDTYGAPGGGNGGRPSDSYGAPGQGQGQGQGQGGYGGKPSDSY |
| | sechellia | GAPGGGNGNGGRPSSSYGAPGGGNGGRPSDTYGAPGGGNGGRPSDTYGAPG |
| | | GGGNGNGGRPSSSYGAPGQGQGNGNGGRPSSSYGAPGGGNGGRPSDTYGAP |
| | | GGGNGGRPSDTYGAPGGGNNGGRPSSSYGAPGGGNGGRPSDTYGAPGGGNG |
| | | NGSGGRPSSSYGAPAQGQGGFGGRPSDSYGAPGQNQKPSDSYGAPGSGNGS |
| | | AGRPSSSYGAPGSGPGGRPSDSYGP |
| 37 | Drosophila | QSGAGGRPSDTYGAPGGGNGGRPSDSYGAPGQGQGQGQGQGGYGGKPSDSY |
| | sechellia | GAPGGGNGNGGRPSSSYGAPGGGNGGRPSDTYGAPGGGNGGRPSDTYGAPG |
| | | GGGNGNGGRPSSSYGAPGQGQGNGNGGRPSSSYGAPGGGNGGRPSDTYGAP |
| | | GGGNGGRPSDTYGAPGGGNNGGRPSSSYGAPGGGNGGRPSDTYGAPGGGNG |
| | | NGSGGRPSSSYGAPAQGQGGFGGRPSDSYGAPGQNQKPSDSYGAPGSGNGS |
| | | AGRPSSSYGAPGSGPGGRPSDSYGP |
| 38 | Drosophila | QSGAGGRPSDTYGAPGGGNGGRPSDSYGAPGQGQGQGQGQGGYGGKPSDSY |
| | sechellia | GAPGGGNGNGGRPSSSYGAPGGGNGGRPSDTYGAPGGGNGGRPSDTYGAPG |
| | | GGGNGNGGRPSSSYGAPGQGQGNGNGGRPSSSYGAPGGGNGGRPSDTYGAP |
| | | GGGNGGRPSDTYGAPGGGNNGGRPSSSYGAPGGGNGGRPSDTYGAPGGGNG |
| | | NGSGGRPSSSYGAPAQGQGGFGGRPSDSYGAPGQNQKPSDSYGAPGSGNGS |
| | | AGRPSSSYGAPGSGPGGRPSDSYGP |
| 39 | Drosophila | YSSGRPGNGNGNGNGGYSSGRPGGQDLGPSGYSGGRPGGQDLGAGGYSNVK |
| | sechellia | PGGQDLGPGGYSGGRPGGQDLGRDGYSGGRPGGQDLGAGAYSNGRPGGNGN |
| | | GGSDGGRVIIGGRVIGGQDGGDQGYSGGRPGGQDLGRDGYSSGRPGGRPGG |
| | | NGQDSQDGQ |
| 40 | Drosophila | YSSGRPGNGNGNGNGGYSSGRPGGQDLGPSGYSGGRPGGQDLGAGGYSNVK |
| | sechellia | PGGQDLGPGGYSGGRPGGQDLGRDGYSGGRPGGQDLGAGAYSNGRPGGNGN |
| | | GGSDGGRVIIGGRVIGGQDGGDQGYSGGRPGGQDLGRDGYSSGRPGGRPGG |
| | | NGQDSQDGQ |
| 41 | Drosophila | YSSGRPGNGNGNGNGGYSSGRPGGQDLGPSGYSGGRPGGQDLGAGGYSNVK |
| | sechellia | PGGQDLGPGGYSGGRPGGQDLGRDGYSGGRPGGQDLGAGAYSNGRPGGNGN |
| | | GGSDGGRVIIGGRVIGGQDGGDQGYSGGRPGGQDLGRDGYSSGRPGGRPGG |
| | | NGQDSQDGQ |
| 42 | Drosophila | YSSGRPGNGNGNGNGGYSSGRPGGQDLGPSGYSGGRPGGQDLGAGGYSNVK |
| | sechellia | PGGQDLGPGGYSGGRPGGQDLGRDGYSGGRPGGQDLGAGAYSNGRPGGNGN |
| | | GGSDGGRVIIGGRVIGGQDGGDQGYSGGRPGGQDLGRDGYSSGRPGGRPGG |
| | | NGQDSQDGQ |
| 43 | Drosophila | YSSGRPGNGNGNGNGGYSSGRPGGQDLGPSGYSGGRPGGQDLGAGGYSNVK |
| | sechellia | PGGQDLGPGGYSGGRPGGQDLGRDGYSGGRPGGQDLGAGAYSNGRPGGNGN |
| | | GGSDGGRVIIGGRVIGGQDGGDQGYSGGRPGGQDLGRDGYSSGRPGGRPGG |
| | | NGQDSQDGQ |
| 44 | Drosophila | YSSGRPGNGNGNGNGGYSSGRPGGQDLGPSGYSGGRPGGQDLGAGGYSNVK |
| | sechellia | PGGQDLGPGGYSGGRPGGQDLGRDGYSGGRPGGQDLGAGAYSNGRPGGNGN |
| | | GGSDGGRVIIGGRVIGGQDGGDQGYSGGRPGGQDLGRDGYSSGRPGGRPGG |
| | | NGQDSQDGQ |
| 45 | 3X FLAG | GDYKDDDDKDYKDDDDKDYKDDDDK |
| 46 | Alpha mating | MRFPSIFTAVLFAASSALAAPVNTTTEDETAQIPAEAVIGYSDLEGDFDVA |
| | factor | VLPFSNSTNNGLLFINTTIASIAAKEEGVSLEKR |
| | precursor | |
| | protein | |
| | sequence | |
| 47 | EA repeat | EAEA |
| 48 | Linker | SG |
| 49 | full-length | MRFPSIFTAVLFAASSALAAPVNTTTEDETAQIPAEAVIGYSDLEGDGDVA |
| | Drosophila | VLPFSNSTNNGLLFINTTIASIAAKEEGVSLEKREAEAgrpeppvnsylpp |
| | sechellia | sdsygapgqsgaggrpsdtygapgggnggrpsdsygapgqgqgqgqgqggy |
| | resilin | ggkpsdsygapgggngnggrpsssygapgggnggrpsdtygapgggnggrp |
| | sequence | sdtygapggggngnggrpsssygapgqgqgngnggrpsssygapgggnggr |
| | (Ds_ACB) | psdtygapgggnggrpsdtygapgggnnggrpsssygapgggnggrpsdty |
| | | gapgggngngsggrpsssygapaqgqggfggrpsdsygapgqnqkpsdsyg |
| | | apgsgngsagrpsssygapgsgpggrpsdsygppasgsgaggaggsgpgga |
| | | dydndepakyefnyqvedapsglsfghsemrdgdfttgqynvllpdgrkqi |
| | | veyeadqqgyrpqiryegdandgsgpsgpsgpggpggqnlgadgyssgrpg |
| | | ngngngnggyssgrpggqdlgpsgysggrpggqdlgaggysnvkpggqdlg |
| | | pggysggrpggqdlgrdgysggrpggqdlgagaysngrpggngnggsdggr |
| | | viiggrviggqdggdqgysggrpggqdlgrdgyssgrpggrpggngqdsqd |
| | | gqgyssgrpgqggrngfgpggqngdndgsgyrySGDYKDDDDKDYKDDDDK |
| | | DYKDDDDK |

1.-58. (canceled)

59. A cosmetic or skincare mask comprising a cross-linked resilin composition, wherein the cross-linked resilin composition comprises a cross-linked recombinant resilin in a polar nonaqueous solvent, wherein the polar nonaqueous solvent comprises glycerol, propylene glycol, ethylene glycol, or DMSO.

60. The cosmetic or skincare mask of claim 59, wherein the polar nonaqueous solvent is selected from the group consisting of glycerol, propylene glycol, and ethylene glycol.

61. The cosmetic or skincare mask of claim 59, wherein the cross-linked resilin composition comprises recombinant resilin in an amount in a range of 20% to 100%, or 30% to 90%, or 40% to 80%, by weight of the cross-linked resilin composition.

62. The cosmetic or skincare mask of claim 59, wherein the cross-linked resilin composition comprises full-length recombinant resilin in an amount of least 20%, at least 50%, at least 70%, at least 80%, or in a range of 10% to 90%, by weight of the cross-linked recombinant resilin.

63. The cosmetic or skincare mask of claim 59, wherein the cross-linked resilin composition does not comprise a cross-linking enzyme or a photocatalyst.

64. The cosmetic or skincare mask of claim 59, wherein the cross-linked resilin composition comprises one or more layers of foam material.

65. The cosmetic or skincare mask of claim 64, wherein the foam material has a porosity of at least 50%.

66. The cosmetic or skincare mask of claim 59, wherein the cross-linked resilin composition further comprises a thickener selected from the group consisting of fumed silica, xanthan gum, and a combination thereof.

67. The cosmetic or skincare mask of claim 59, wherein the cross-linked resilin composition further comprises a hardener selected from the group consisting of aliphatic polyamines, fatty polyamides, aromatic polyamines hardeners, anhydride hardeners, boron trifluoride hardeners, dicyandiamide curing agents, and combinations thereof.

68. The cosmetic or skincare mask of claim 59, wherein the cross-linked resilin composition has a hardness of at least 10, or in a range of 10 to 50, as measured using a Shore 00 Durometer in accordance with ASTM D2240.

69. The cosmetic or skincare mask of claim 59, wherein the cross-linked resilin composition has a density in a range of 0.5 mg/cm³ to 2.0 mg/cm³, or 1.0 mg/cm³ to 1.5 mg/cm³, or 1.1 mg/cm³ to 1.4 mg/cm³, or 1.2 mg/cm³ to 1.35 mg/cm³, as measured in accordance with ASTM D297-15.

70. The cosmetic or skincare mask of claim 59, wherein the cosmetic or skincare mask is in the form of a facial mask.

71. A cosmetic or skincare composition comprising a cross-linked resilin composition, wherein the cross-linked resilin composition comprises a solid cross-linked recombinant resilin in a polar nonaqueous solvent, wherein the polar nonaqueous solvent comprises glycerol, propylene glycol, ethylene glycol, or DMSO.

72. The cosmetic or skincare composition of claim 71, wherein the polar nonaqueous solvent is selected from the group consisting of glycerol, propylene glycol, and ethylene glycol.

73. The cosmetic or skincare composition of claim 71, wherein the cosmetic or skincare composition is in the form of a cream, foam, gel, stick, mousse, milk, spray, or lotion.
